(19) United States
(12) Patent Application Publication
(10) Pub. No.: US 2023/0190911 A1
(43) Pub. Date: Jun. 22, 2023

(54) VACCINE FORMULATIONS WITH INCREASED STABILITY

(71) Applicant: VAXESS TECHNOLOGIES, INC., Cambridge, MA (US)

(72) Inventors: Kathryn M. Kosuda, Cambridge, MA (US); David P. Miller, Walpole, MA (US); Nishant K. Jain, San Diego, CA (US); Carter R. Palmer, La Jolla, CA (US); Jonathan A. Kluge, Cambridge, MA (US); Jordan A. Stinson, Somerville, MA (US); Adrian Benton Li, Brighton, MA (US); Alexandra Krisiewicz, Morris Plains, NJ (US)

(73) Assignee: VAXESS TECHNOLOGIES, INC., Cambridge, MA (US)

(21) Appl. No.: 17/958,148

(22) Filed: Sep. 30, 2022

Related U.S. Application Data
(63) Continuation of application No. 16/334,215, filed on Mar. 18, 2019, now abandoned.
(60) Provisional application No. 62/486,796, filed on Apr. 18, 2017.
(60) Provisional application No. 62/403,873, filed on Oct. 4, 2016.
(60) Provisional application No. 62/403,886, filed on Oct. 4, 2016.
(60) Provisional application No. 62/396,575, filed on Sep. 19, 2016.
(60) Provisional application No. 62/396,560, filed on Sep. 19, 2016.

(51) Int. Cl. A61K 39/12; A61P 31/14; A61K 39/13; A61K 39/15; A61K 47/02; A61K 47/26; A61K 47/42; A61K 39/00

(52) U.S. Cl. CPC A61K 39/12; A61P 31/14; A61K 39/13; A61K 39/15; A61K 47/02; A61K 47/26; A61K 47/42; A61K 39/00; C12N 2720/12334; C12N 2770/24134; C12N 2770/32334; C12N 2770/32634; Y02A 50/30

(57) ABSTRACT

The present disclosure relates to viral vaccine formulations with enhanced stability and methods of use thereof.

This application is a continuation application of U.S. patent application Ser. No. 16/334,215 filed Mar. 18, 2019, which is the 371 National Stage application of International Patent Application No. PCT/US2017/052301 filed Sep. 19, 2017, which claims priority to U.S. Ser. No. 62/403,873 filed Oct. 4, 2016, U.S. Ser. No. 62/403,886 filed Oct. 4, 2016, U.S. Ser. No. 62/396,575 filed Sep. 19, 2016, U.S. Ser. No. 62/396,560 filed Sep. 19, 2016, and U.S. Ser. No. 62/486,796 filed Apr. 18, 2017, the contents of all of which are incorporated herein by reference in their entireties.

### BACKGROUND

### Enteroviruses

Enteroviruses are a genus of single-stranded positive-sense RNA viruses within the picornavirus family. The enteroviruses were originally classified into four groups: polioviruses (PV), Coxsackie A viruses (CV-A), Coxsackie B viruses (CV-B), and echoviruses (E). These classes, which were based on pathogenic properties, were later superseded by twelve species (Enterovirus (EV) A, B, C, D, E, F, G, H and J, and Human Rhinovirus (HRV) A, B and C) defined by genetic analyses. Currently, there are over 70 serotypes of human enteroviruses, which are designated by a system with consecutive numbers: PV-1, PV-2, PV-3, etc., CV-A1, CV-A2, CV-A3, etc., CV-B1, CV-B2, CV-B3, etc., E-1, E-2, E-3, etc., EV-1, EV-2, EV-3, etc., HRV-A1, HRV-A2, HRV-A3, etc., HRV-B1, HRV-B2, HRV-B3, etc., and HRV-C1, HRV-C2, HRV-C3, etc., (see, Oberste et al. (1999), J. Virol. 73(3): 1941-8; Nasri et al. (2007), Expert Rev. Mol. Diagn. 7(4):419-34).Poliovirus (PV), the causative agent of poliomyelitis (commonly known as polio), is a human enterovirus. Poliovirus infection occurs via the fecal-oral route, meaning that one ingests the virus and viral replication occurs in the alimentary tract. Virus is shed in the feces of infected individuals. In 95% of cases only a primary, transient presence of viremia (virus in the bloodstream) occurs, and the poliovirus infection is asymptomatic. In about 5% of cases, the virus spreads and replicates in other sites such as brown fat, reticuloendothelial tissue, and muscle. The sustained viral replication causes secondary viremia and leads to the development of minor symptoms such as fever, headache, and sore throat. Paralytic poliomyelitis occurs in less than 1% of poliovirus infections. Paralytic disease occurs when the virus enters the central nervous system (CNS) and replicates in motor neurons within the spinal cord, brain stem, or motor cortex, resulting in the selective destruction of motor neurons leading to temporary or permanent paralysis. In rare cases, paralytic poliomyelitis leads to respiratory arrest and death. In cases of paralytic disease, muscle pain and spasms are frequently observed prior to onset of weakness and paralysis. Paralysis typically persists anywhere from days to weeks prior to recovery.

Polio was one of the most dreaded childhood diseases of the 20th century in the United States. Periodic epidemics occurred since the late 19th century and they increased in size and frequency in the late 1940s and early 1950s. An average of over 35,000 new cases per year were reported during this time period. With the introduction of Salk inactivated polio vaccine (IPV) in 1955, the number of cases rapidly declined to under 2,500 cases in 1957. The Sabin oral polio vaccine, which consisted of live attenuated versions of the three serotypes of poliovirus, was introduced in 1961. By 1965, only 61 cases of paralytic polio were reported. The last cases of naturally occurring paralytic polio in the United States were in 1979, when an outbreak occurred in several Midwestern states.

Worldwide, about 99% of polio cases have been eradicated. However, tackling the last 1% of polio cases has still proved to be difficult. Conflict, political instability, hard-to-reach populations, and poor infrastructure continue to pose challenges to eradicating the disease.

While poliomyelitis has historically been the most significant enterovirus-caused disease, there are a number of non-polio enteroviruses that can cause disease in humans. These include Coxsackie A viruses, Coxsackie B viruses, echoviruses, and rhinoviruses. These viruses cause diseases ranging from the common cold to hand, foot, and mouth disease.

Enteroviruses share similar structural properties. Enterovirus virions are approximately 30 nm in diameter and roughly spherical. They do not have lipid envelopes, and their capsids are composed of 60 copies of each of four proteins arranged with icosahedral symmetry around the RNA genome.

### Rotaviruses

Rotaviruses are a genus of double-stranded RNA viruses within the Reoviridae family. Rotavirus virions are non-enveloped, roughly 100 nm in diameter, and have triple-layered capsids that surround a genome of 11 segments of viral RNA encoding for 6 structural (VP1-VP4, VP6, and VP7) and 6 non-structural (NSP1-NSP6) proteins. Rotaviruses are divided into eight groups (A-H) based on genetic and antigenic differences in the VP6 protein, and further classified by serotype and/or genotype based on their VP7 (G type) and VP4 (P type) proteins. There are at least 27 G serotypes and 37 P genotypes, but group A rotaviruses of five G serotypes (G1-G4 and G9) and three P genotypes (P[4], P[6], and P[8]) cause most of the human rotavirus infections globally, with G1P[8] being the most common infection-causing strain, followed by G3P[8], G2P[4], G9P[8], and G4P[8]. (See, e.g., Yen and Cortese, “Rotaviruses,” in Principles and Practice of Pediatric Infectious Diseases, 4^{th} ed., Long et al., Eds., 2012, Elsevier, London; Gastanaduy and Begue, “Acute Gastroenteritis Vaccines,” in Infectious Diseases, 3^{rd} ed., Cohen et al., Eds., 2010, Elsevier, London; Angel et al., “Rotavirus Infections,” in Tropical Infectious Diseases: Principles, Pathogens and Practice, 3^{rd} ed., Guerrant et al., Eds., 2011, Elsevier, London.)

Rotavirus is transmitted primarily via the fecal-oral route, including through person-to-person contact and contaminated food or surfaces. It is extremely contagious due to the large number of viral particles typically excreted in feces (˜10¹² virions per mL) and the low dose typically required to transmit infection (˜10⁴ virions) (Gastanaduy and Begue (2010), supra). Rotavirus infections attack cells lining the small intestine, in particular mature enterocytes on the tips of small intestinal villi, destroying their absorptive capacity and causing diarrhea. Severe cases can result in diarrhea, vomiting, dehydration, malnutrition, and death. And unlike other types of diarrhea, rotaviral gastroenteritis cannot be controlled through improvements in hygiene and sanitation, as rotavirus is so contagious that such efforts are relatively ineffective. (See, e.g., Global Alliance for Vaccines (GAVI) website.)

Acute diarrhea is the second most common cause of mortality in children up to five years old worldwide, and rotaviruses are in turn the leading cause of diarrhea in that population (Gastanaduy and Begue (2010), supra). The World Health Organization estimates that approximately 453,000 children died from rotaviral gastroenteritis in 2008, accounting for about 5% of all child deaths (World Health Organization, Weekly Epidemiological Record, No. 5, 2013, 88:49-64). Prior to the introduction of rotavirus vaccine in 2006, rotavirus caused 3.5 million cases of infection, 55,000 hospitalizations, and up to 40 deaths each year in the United States alone (Gastanaduy and Begue (2010), supra).

### Flaviviruses

Flavivirus is a genus of viruses in the family Flaviviridae. This genus includes many disease-causing viruses, such as the West Nile virus, dengue virus, Zika virus, tick-borne encephalitis virus, yellow fever virus, and several other viruses that may cause encephalitis (e.g.,

Japanese encephalitis). Flaviviruses share several common aspects: common size (40-65 nm), symmetry (enveloped, icosahedral nucleocapsid), nucleic acid (positive-sense, single-stranded RNA of approximately 10,000-11,000 bases), and appearance in the electron microscope.

Viral infections caused by flaviviruses are generally transmitted by the bite from an infected arthropod (mosquito or tick). No specific antiviral therapies are currently available for the diseases caused by insect-vectored flaviviruses. Thus, efforts have been focused on the prevention of disease, through either vaccination or vector control, rather than on the treatment of infected individuals. While vector control can occasionally be successful in controlling the spread of flavivirus outbreaks, vaccines appear to be a more cost-effective, sustainable, and environmentally friendly approach. A review of vaccines for the medically important flaviviruses presents the full spectrum of vaccine options and complexity levels, and provides examples of successes and major challenges. The insect-borne flavivirus vaccine field is dynamic, with new and improved vaccines being advanced.

### Effectiveness of Vaccine Formulations

Almost all current vaccine products, including enterovirus vaccines, such as oral polio vaccine (OPV) and inactivated polio vaccine (IPV), currently marketed rotavirus vaccines, and flavivirus vaccines, such as yellow fever vaccine, Japanese encephalitis vaccine, and dengue vaccine, are sensitive to both freezing and elevated temperatures, and therefore are preferably shipped and stored between 2 and 8° C., a requirement that imposes financial and logistical challenges in the global distribution of vaccines. Breaks in the “cold chain” (i.e., continuous maintenance of the vaccine at temperatures between 2 and 8° C.) are common and result in vaccine wastage and risk of ineffective vaccine administration. Thermostable vaccine formulations would simplify access to areas of the world that lack sufficient cold-chain capacity and decrease cold-chain-associated costs for vaccine manufacturers, national governments, and non-profit vaccine buyers.

Removing enterovirus vaccines, including IPV, rotavirus vaccines, and flavivirus vaccines from the constraints of the cold chain and/or improving the post-reconstitution stability of such vaccines would make a significant contribution to the global effort to control (e.g., eradicate) enteroviruses, rotavirus, and or flavivirus spread and infection by reducing costs and simplifying logistics related to cold storage and vaccine spoilage.

Therefore, there exists a need for dried and liquid vaccine formulations for preventing infections caused by enteroviruses, including but not limited to poliovirus, rotaviruses, and flaviviruses, including but not limited to yellow fever virus, Japanese encephalitis virus, dengue virus, and Zika virus, that have increased temperature stability.

### SUMMARY OF THE INVENTION

The present invention discloses, at least in part, viral vaccine preparations with surprisingly increased stability over time and/or at elevated temperatures. In some embodiments, the vaccine preparations are substantially dry. In other embodiments, the vaccine preparations are in liquid form. In some embodiments, the vaccine preparations include a viral immunogen, a protein excipient (also referred to interchangeably herein as a “protein stabilizer”), and a sugar or sugar alcohol excipient. The vaccine preparations can be produced by forming a solution of the vaccine antigen with a protein excipient, and substantially drying the resulting solution by a techniques including lyophilization, vacuum-drying, and/or air-drying. Thus, optimized vaccine preparations, methods of making and using are disclosed.

Accordingly, in one aspect, the invention provides a substantially dried viral vaccine preparation. In some embodiments, the vaccine preparation includes a viral immunogen; a protein excipient, e.g., a protein excipient selected from the group consisting of a silk fibroin, a gelatin and an albumin, or a combination thereof; a sugar or a sugar alcohol excipient, e.g., a sugar or sugar alcohol excipient selected from the group consisting of a sucrose, a trehalose, a sorbitol and a glycerol, or a combination thereof; and optionally, a divalent cation. In some embodiments, the vaccine preparation has one, two, three, or four of the following properties:

(i) retains at least 30%, 40%, or 50% of its original bioactivity after storage at 40-45° C. for 3-6 months;

(ii) retains at least 30%, 40%, or 50% of its original bioactivity after storage at 45° C. for 4, 8 or 12 weeks;

(iii) retains at least 30%, 40%, 50% or 60% of its original bioactivity after storage at 37° C. for 4, 8 or 12 weeks; or

(iv) retains at least 70%, 80% or 90% of its original bioactivity after storage at 25° C. for 4, 8, or 12 weeks. In some embodiments, when (i)-(iv) are tested in the vaccine preparation comprising the protein excipient present in an amount of less than 4% (w/v), optionally, between about 2% (w/v) and about 2.5% (w/v), immediately before drying.

In some embodiments, the viral immunogen is selected from the group consisting of an enterovirus immunogen, a flavivirus immunogen, a rotavirus immunogen, a measles virus immunogen, a mumps virus immunogen, a rubella virus immunogen, and an influenza virus immunogen. In other embodiments, the viral immunogen is selected from the group consisting of an enterovirus immunogen, a flavivirus immunogen, and a rotavirus immunogen.

In some embodiments, the substantially dried viral vaccine preparation contains water in an amount between 5% and 20%, or in an amount between 0% and 5%. In some embodiments, the substantially dried viral vaccine preparation contains water in an amount 4.7% or greater, e.g., 4.7% to 10%.

In some embodiments, the substantially dried viral vaccine preparation is prepared by air drying, vacuum drying, or lyophilization, e.g., partial lyophilization. In some embodiments, the substantially dried viral vaccine is prepared by vacuum drying. In some embodiments, the substantially dried viral vaccine is prepared by lyophilization, e.g., partial lyophilization. In some embodiments, the substantially dried viral vaccine preparation (e.g., a large-scale substantially dried viral vaccine preparation) is prepared by air drying at about 2° C. to about 50° C. (e.g., at about 20° C. to about 25° C. and at about 20% to about 40% relative humidity). In some embodiments, a large-scale formulation is prepared in an amount greater than about 1-million dosage units per year (e.g., between about 1-million to about 2-million dosage units per year).

In some embodiments, the substantially dried viral vaccine preparation is a large-scale substantially dried viral vaccine preparation, e.g., in an amount greater than about 1-million dosage units per year (e.g., between about 1-million to about 2-million dosage units per year).

In some embodiments, the protein excipient is the silk fibroin present in an amount less than 10% (w/v), less than 9% (w/v), less than 8% (w/v), less than 7% (w/v), less than 6% (w/v), less than 5% (w/v), less than 4% (w/v), less than 3.5% (w/v), less than 3% (w/v), less than 2.5% (w/v), less than 2% (w/v), less than 1.5% (w/v), less than 1% (w/v), less than 0.5% (w/v), less than 0.1% (w/v), but greater than 0.001% (w/v), immediately before drying. In some embodiments, silk fibroin is present in an amount between about 1% (w/v) to about 3% (w/v), about 1.5% (w/v) to about 2.8% (w/v), or about 2% (w/v) and about 2.5% (w/v), e.g., immediately before drying.

In some embodiments, the protein excipient is gelatin present in an amount between about 1% (w/v) to about 10% (w/v), about 2% (w/v) to about 8% (w/v), or about 4% (w/v) and about 6% (w/v), about 1% (w/v) to about 3% (w/v), about 1.5% (w/v) to about 2.8% (w/v), or about 2% (w/v) and about 2.5% (w/v), e.g., immediately before drying.

In some embodiments, the protein excipient is albumin present in an amount between about 0.1% (w/v) to about 10% (w/v), about 0.2% (w/v) to about 8% (w/v), or about 0.4% (w/v) and about 6% (w/v), about 0.5% (w/v) to about 3% (w/v), about 0.6% (w/v) to about 2.8% (w/v), about 0.8% (w/v) and about 2.5%, or about 0.1%, or about 2.4% (w/v), e.g., immediately before drying.

In some embodiments, the sugar or the sugar alcohol is sucrose present in an amount less than 70% (w/v), less than 60% (w/v), less than 50% (w/v), less than 40% (w/v), less than 30% (w/v), less than 20% (w/v), less than 10% (w/v), less than 9% (w/v), less than 8% (w/v), less than 7% (w/v), less than 6% (w/v), or 5% (w/v) or less, e.g., immediately before drying.

In some embodiments, the sugar or the sugar alcohol is sucrose present in an amount between about 1% (w/v) to about 10% (w/v), about 2% (w/v) to about 8% (w/v), about 2.2% (w/v) to about 6% (w/v), about 2.4% (w/v) to about 5.5% (w/v), about 2.5 to about 5%, or about 2.4% (w/v), about 2.5%, or about 5% (w/v), e.g., immediately before drying.

In some embodiments, the sugar or the sugar alcohol is trehalose present in an amount between about 1% (w/v) to about 10% (w/v), about 2% (w/v) to about 8% (w/v), about 2.2% (w/v) to about 6% (w/v), about 2.4% (w/v) to about 5.5% (w/v), about 2.5 to about 5%, or about 2.4% (w/v), about 2.5%, or about 5% (w/v), e.g., immediately before drying.

In some embodiments, the sugar or the sugar alcohol is sorbitol present in an amount between about 1% (w/v) to about 10% (w/v), about 2% (w/v) to about 8% (w/v), about 2.2% (w/v) to about 6% (w/v), about 2.4% (w/v) to about 5.5% (w/v), about 2.5 to about 5%, or about 2.4% (w/v), about 2.5%, or about 5% (w/v), e.g., immediately before drying.

In some embodiments, the sugar or the sugar alcohol is glycerol present in an amount between about 1% (w/v) to about 10% (w/v), about 2% (w/v) to about 8% (w/v), about 2.2% (w/v) to about 6% (w/v), about 2.4% (w/v) to about 5.5% (w/v), about 2.5 to about 5%, or about 2.4% (w/v), about 2.5%, or about 5% (w/v), e.g., immediately before drying.

In some embodiments, the substantially dried viral vaccine preparation further comprising a divalent cation. In some embodiments, the divalent cation is selected from the group consisting of Ca²⁺, Mg²⁺, Mn²⁺, and Cu²⁺. In some embodiments, the divalent cation is present in the preparation immediately before drying in an amount between 0.1 mM and 100 mM. In some embodiments, the divalent cation is present in the preparation immediately before drying in an amount between 10⁻⁷ and 10⁻⁴ moles per standard dose of viral immunogen. In some embodiments, the divalent cation is present in the preparation immediately before drying in an amount between 10⁻¹⁰ to 2×10⁻³ moles.

In some embodiments, the substantially dried viral vaccine preparation further comprising a buffer, e.g., immediately before drying. In some embodiments, the buffer has buffering capacity between pH 3 and pH 8, between pH 4 and pH 7.5, or between pH 5 and pH 7. In some embodiments, the buffer is selected from the group consisting of HEPES and a CP buffer. In some embodiments, the buffer is present in the preparation immediately before drying in an amount between 0.1 mM and 100 mM. In some embodiments, the buffer is present in an amount between 10⁻⁷ and 10⁻⁴ moles per standard dose of viral immunogen. In some embodiments, the buffer is present in an amount between 10⁻¹⁰ to 2×10⁻³ moles.

In some embodiments, the viral immunogen is an enterovirus immunogen. In some embodiments, the viral immunogen is a flavivirus immunogen. In some embodiments, the viral immunogen is a rotavirus immunogen. In some embodiments, the viral immunogen is a measles virus. In some embodiments, the viral immunogen is a mumps virus. In some embodiments, the viral immunogen is a rubella virus. In other embodiments, the viral immunogen is not a measles virus, a mumps virus, and/or a rubella virus. In some embodiments, the viral immunogen is an influenza virus.

In one aspect, the invention provides a method of treating or preventing an infection caused by a virus. The method includes administering to a subject in need thereof an effective amount of a vaccine preparation as described herein, to treat or prevent the infection.

In one aspect, the invention provides a method of eliciting an immune response to a virus in a subject. The method includes administering to a subject in need thereof a vaccine preparation as described herein in an amount sufficient to elicit the immune response to the virus.

In some embodiments of the methods, the subject is selected from a human and a non-human mammal. In some embodiments, the subject is an adult or a child. In some embodiments, the vaccine preparation is administered by a route selected from the group consisting of oral, subcutaneous, dermal (e.g., transdermal, intradermal or interdermal) and intramuscular.

### Enterovirus

The present invention discloses, at least in part, substantially dry enterovirus vaccine preparations with surprisingly increased stability over time and/or at elevated temperatures. In some embodiments, the entrovirus vaccine preparation includes an enterovirus immunogen, a protein excipient (also referred to interchangeably herein as a “protein stabilizer”), and a sugar or sugar alcohol excipient. In some embodiments, the enterovirus vaccine preparation can further comprise a divalent cation. The enterovirus vaccine preparation can be produced by forming a solution of the vaccine antigen with a protein excipient, and substantially drying the resulting solution by a techniques including lyophilization, vacuum-drying, and/or air-drying.

Thus, in certain embodiments, the invention provides a substantially dried, stabilized vaccine formulation comprising an enterovirus immunogen (such as IPV or an inactivated coxsackie virus or rhinovirus), a protein stabilizer, a sugar or sugar alcohol excipient, and, optionally, a divalent cation. In certain embodiments, the stabilized vaccine formulation retains significant bioactivity when stored at 37° C. or 45° C. for at least six months. In certain embodiments, the stabilized vaccine formulation retains significant bioactivity when stored at 20° C. or 25° C. for up to two years. In certain embodiments, the enterovirus vaccine preparation has one, two, three, or four of the following properties: (i) retains at least 30%, 40%, or 50% of its original bioactivity after storage at 40-45° C. for 3-6 months, (ii) retains at least 30%, 40%, or 50% of its original bioactivity after storage at 45° C. for 4, 8 or 12 weeks; (iii) retains at least 30%, 40%, 50% or 60% of its original bioactivity after storage at 37° C. for 4, 8 or 12 weeks; or (iv) retains at least 70%, 80% or 90% of its original bioactivity after storage at 25° C. for 4, 8, or 12 weeks, e.g., when (i)-(iv) are tested in the vaccine preparation comprising the protein excipient present in an amount of less than 4% (w/v), optionally, between about 2% (w/v) and about 2.5% (w/v), immediately before drying.

Thus, in one aspect, the invention provides a substantially dried enterovirus vaccine preparation comprising: an enterovirus immunogen; a protein excipient; and a sugar or sugar alcohol excipient. In some embodiments, the enterovirus is selected from a polio virus, a coxsackie virus, a human rhinovirus and an echo virus. In some embodiments, the enterovirus immunogen is selected from the group consisting of a live attenuated enterovirus and an inactivated virus. In some specific embodiments, the enterovirus immunogen comprises at least one inactivated poliovirus (IPV), and in some cases PV-1, PV-2 or PV-3.

In some embodiments, the enterovirus immunogen is present in any amount between 0.001 and 20 standard doses. In some embodiments, an IPV immunogen is present in an amount between 0.04 and 800 D-antigen units for inactivated Type 1 poliovirus, between 0.008 and 1000 D-antigen units for inactivated Type 2 poliovirus, or between 0.032 and 1280 D-antigen units for inactivated Type 3 poliovirus.

In some embodiments, the protein excipient is selected from a silk fibroin, a gelatin and an albumin, or a combination thereof.

In some embodiments, the protein excipient is present in the formulation immediately before drying in an amount between 0.1% and 10% (w/v). In some embodiments, the protein excipient is present in the formulation before, e.g., immediately before, drying in an amount between 0.25% and 7.5% (w/v). In some embodiments, the protein excipient is present in the formulation before, e.g., immediately before, drying in an amount between 0.5% and 5% (w/v). In some embodiments, the protein excipient is present in the formulation before, e.g., immediately before, drying in an amount between 1% and 5% (w/v).

In some embodiments, the protein excipient is present in an amount between 1.0 mg and 100 mg per standard dose of enterovirus immunogen. In some embodiments, the protein excipient is present in an amount between 2.5 mg and 75 mg per standard dose of enterovirus immunogen. In some embodiments, the protein excipient is present in an amount between 5.0 mg and 50 mg per standard dose of enterovirus immunogen. In some embodiments, the protein excipient is present in an amount between 10 mg and 50 mg per standard dose of enterovirus immunogen.

In some embodiments, the protein excipient is present in an amount between 0.001 mg and 2 g. In some embodiments, the protein excipient is present in an amount between 0.0025 mg and 1.5 g. In some embodiments, the protein excipient is present in an amount between 0.005 mg and 1 g. In some embodiments, the protein excipient is present in an amount between 0.01 mg and 1 g. In some embodiments, the protein excipient is present in an amount between 1.0 mg and 100 mg. In some embodiments, the protein excipient is present in an amount between 2.5 mg and 75 mg. In some embodiments, the protein excipient is present in an amount between 5.0 mg and 50 mg. In some embodiments, the protein excipient is present in an amount between 10 mg and 50 mg.

In some embodiments, the sugar or sugar alcohol excipient is selected from a sucrose, a trehalose, a sorbitol and a glycerol, or a combination thereof.

In some embodiments, the sugar or sugar alcohol excipient is present in the formulation before, e.g., immediately before, drying in an amount between 0.1% and 50% (w/v). In some embodiments, the sugar or sugar alcohol excipient is present in the formulation before, e.g., immediately before, drying in an amount between 0.5% and 25% (w/v). In some embodiments, the sugar or sugar alcohol excipient is present in the formulation before, e.g., immediately before, drying in an amount between 0.5% and 10% (w/v). In some embodiments, the sugar or sugar alcohol excipient is present in the formulation before, e.g., immediately before, drying in an amount between 1% and 10% (w/v).

In some embodiments, the sugar or sugar alcohol excipient is present in an amount between 1.0 mg to 500 mg per standard dose of enterovirus immunogen. In some embodiments, the sugar or sugar alcohol excipient is present in an amount between 5.0 mg and 250 mg per standard dose of enterovirus immunogen. In some embodiments, the sugar or sugar alcohol excipient is present in an amount between 5.0 mg and 100 mg per standard dose of enterovirus immunogen. In some embodiments, the sugar or sugar alcohol excipient is present in an amount between 10 mg and 100 mg per standard dose of enterovirus immunogen.

In some embodiments, the sugar or sugar alcohol excipient is present in an amount between 0.001 mg and 10 g. In some embodiments, the sugar or sugar alcohol excipient is present in an amount between 0.005 mg and 5.0 g. In some embodiments, the sugar or sugar alcohol excipient is present in an amount between 0.005 mg and 2 g. In some embodiments, the sugar or sugar alcohol excipient is present in an amount between 0.01 mg and 2 g. In some embodiments, the sugar or sugar alcohol excipient is present in an amount between 1.0 mg to 500 mg. In some embodiments, the sugar or sugar alcohol excipient is present in an amount between 5.0 mg and 250 mg. In some embodiments, the sugar or sugar alcohol excipient is present in an amount between 5.0 mg and 100 mg. In some embodiments, the sugar or sugar alcohol excipient is present in an amount between 10 mg and 100 mg.

In some embodiments, the divalent cation is selected from the group consisting of Ca²⁺, Mg²⁺, Mn²⁺, and Cu²⁺.

In some embodiments, the divalent cation is present in the formulation before, e.g., immediately before, drying in an amount between 0.1 mM and 100 mM. In some embodiments, the divalent cation is present in the formulation before, e.g., immediately before, drying in an amount between 1 mM and 100 mM. In some embodiments, the divalent cation is present in the formulation before, e.g., immediately before, drying in an amount between 0.5 mM and 50 mM.

In some embodiments, the divalent cation is present in an amount between 10⁻⁷ and 10⁻⁴ moles per standard dose of enterovirus immunogen. In some embodiments, the divalent cation is present in an amount between 10⁻⁶ and 10⁻⁴ moles per standard dose of enterovirus immunogen. In some embodiments, the divalent cation is present in an amount between 5×10⁻⁶ and 5×10⁻⁵ moles per standard dose of enterovirus immunogen.

In some embodiments, the divalent cation is present in an amount between 10⁻¹⁰ and 2×10⁻³ moles. In some embodiments, the divalent cation is present in an amount between 10⁻⁹ and 2×10⁻³ moles. In some embodiments, the divalent cation is present in an amount between 5×10⁻⁹ and 10⁻³ moles. In some embodiments, the divalent cation is present in an amount between 10⁻⁷ and 10⁻⁴ moles. In some embodiments, the divalent cation is present in an amount between 10⁻⁶ and 10⁻⁴ moles. In some embodiments, the divalent cation is present in an amount between 5×10⁻⁶ and 5×10⁻⁵ moles.

In some embodiments, the buffer has buffering capacity between pH 3 and pH 8, between pH 4 and pH 7.5, or between pH 5 and pH 7. In some embodiments, the buffer is selected from the group consisting of HEPES and a CP buffer.

In some embodiments, the buffer is present in the formulation before, e.g., immediately before, drying in an amount between 0.1 mM and 100 mM. In some embodiments, the buffer is present in the formulation before, e.g., immediately before, drying in an amount between 1 mM and 100 mM. In some embodiments, the buffer is present in the formulation before, e.g., immediately before, in an amount between 0.5 mM and 50 mM.

In some embodiments, the buffer is present in an amount between 10⁻⁷ and 10⁴ moles per standard dose of enterovirus immunogen. In some embodiments, the buffer is present in an amount between 10⁻⁶ and 10⁻⁴ moles per standard dose of enterovirus immunogen. In some embodiments, the buffer is present in an amount between 5×10⁻⁶ and 5×10⁻⁵ moles per standard dose of enterovirus immunogen.

In some embodiments, the buffer is present in an amount between 10⁻¹⁰ and 2×10⁻³ moles. In some embodiments, the buffer is present in an amount between 10⁻⁹ and 2×10⁻³ moles. In some embodiments, the buffer is present in an amount between 5×10⁻⁹ and 10⁻³ moles. In some embodiments, the buffer is present in an amount between 10⁻⁷ and 10⁻⁴ moles. In some embodiments, the buffer is present in an amount between 10⁻⁶ and 10⁻⁴ moles. In some embodiments, the buffer is present in an amount between 5×10⁻⁶ and 5×10⁻⁵ moles.

In some embodiments, the preparation is dried by a process selected from the group consisting of air-drying, vacuum drying and lyophilization. In some embodiments, the preparation comprises water in an amount between 0% and 5%, and in some of those embodiments, the preparation is produced by lyophilization. In some embodiments, the preparation comprises water in an amount between 5% and 20%, and in some of those embodiments, the preparation is produced by air-drying.

In some embodiments, the preparation retains at least 70%, 80% or 90% of its original bioactivity after storage at 25° C. for 2 weeks; at least 70%, 80% or 90% of its original bioactivity after storage at 25° C. for 4 weeks; at least 70%, 80% or 90% of its original bioactivity after storage at 25° C. for 8 weeks; and/or at least 70%, 80% or 90% of its original bioactivity after storage at 25° C. for 12 weeks.

In some embodiments, the preparation retains at least 60%, 70%, or 80% of its original bioactivity after storage at 37° C. for 2 weeks; at least 60%, 70%, or 80% of its original bioactivity after storage at 37° C. for 4 weeks; at least 50%, 60%, or 70% of its original bioactivity after storage at 37° C. for 8 weeks; and/or at least 30%, 40%, or 50% of its original bioactivity after storage at 37° C. for 12 weeks.

In some embodiments, the preparation retains at least 50%, 60%, or 70% of its original bioactivity after storage at 45° C. for 2 weeks; at least 30%, 40%, or 50% of its original bioactivity after storage at 45° C. for 4 weeks; at least 30%, 40%, or 50% of its original bioactivity after storage at 45° C. for 8 weeks; and/or at least 30%, 40%, or 50% of its original bioactivity after storage at 45° C. for 12 weeks.

In another aspect, the invention provides a method of treating or preventing an infection caused by an enterovirus, by administering to a subject in need thereof a therapeutically or prophylactically effective amount of a vaccine preparation of the invention, thereby eliciting an immune response in the subject and treating or preventing the infection.

In one aspect, the invention provides a method of eliciting an immune response to a virus in a subject. The method includes administering to a subject in need thereof an enterovaccine preparation as described herein in an amount sufficient to elicit the immune response to the virus.

In some embodiments, the subject is selected from a human and a non-human mammal. In some embodiments, the subject is an adult or a child. In some embodiments, the vaccine is administered by a route selected from oral, subcutaneous, dermal (e.g., transdermal, intradermal or interdermal), and intramuscular.

These and other embodiments of the invention are described in the following figures, detailed description and claims.

### Flavivirus

The present invention discloses, at least in part, a flavivirus vaccine preparation with surprisingly increased stability over time and/or at elevated temperatures. In some embodiments, the flavivirus vaccine preparation is a liquid formulation. In some embodiments, the liquid flavivirus vaccine preparation comprises a protein stabilizer (also interchangeably referred to herein as a “protein excipient”). The liquid preparation can be provided by forming a solution of the vaccine immunogen with a certain protein stabilizer. In other embodiments, the flavivirus vaccine preparation is a substantially dried formulation and includes the flavivirus immunogen, a protein excipient and a sugar or sugar alcohol excipient. The substantially dried preparation can be provided by forming a solution of the vaccine immunogen with a certain protein stabilizer and a sugar or sugar alcohol excipient and then drying the resulting solution by a technique such as lyophilization, vacuum-drying, and/or air-drying.

Thus, in one aspect, the invention provides a liquid stabilized flavivirus vaccine preparation comprising a flavivirus immunogen and a protein stabilizer.

In some embodiments, the flavivirus immunogen is selected from the group consisting of a live attenuated flavivirus, an inactivated flavivirus, a chimeric flavivirus, and a recombinant flavivirus immunogen. In some embodiments, the flavivirus is chosen from a yellow fever virus, a Japanese encephalitis virus, a dengue virus, and a Zika virus. In some embodiments, the flavivirus immunogen is present in any amount between 0.001 and 20 standard doses.

In some embodiments, the protein stabilizer is selected from the group consisting of a silk fibroin, an albumin, a gelatin, or a combination thereof.

In some embodiments, the silk fibroin is present in an amount from 0.1% (w/v) to 20% (w/v). In some embodiments, the albumin is present in an amount from 0.01% (w/v) to 10% (w/v). In some embodiments, the gelatin is present in an amount over 1.5% (w/v) and up to 10% (w/v).

In some embodiments, the stabilized liquid flavivirus vaccine preparation retains at least 50% of its original bioactivity after storage at 4° C. for 4 weeks, at least 50% of its original bioactivity after storage at 25° C. for 48 hours, and/or at least 50% of its original bioactivity after storage at 37° C. for 8 hours.

In another aspect, the invention provides a substantially dried stabilized flavivirus vaccine preparation comprising a flavivirus immunogen, a protein stabilizer and a sugar or sugar alcohol excipient. In certain embodiments, the flavivirus vaccine preparation has one, two, three, or four of the following properties: (i) retains at least 30%, 40%, or 50% of its original bioactivity after storage at 40-45° C. for 3-6 months, (ii) retains at least 30%, 40%, or 50% of its original bioactivity after storage at 45° C. for 4, 8 or 12 weeks; (iii) retains at least 30%, 40%, 50% or 60% of its original bioactivity after storage at 37° C. for 4, 8 or 12 weeks; or (iv) retains at least 70%, 80% or 90% of its original bioactivity after storage at 25° C. for 4, 8, or 12 weeks, e.g., when (i)-(iv) are tested in the vaccine preparation comprising the protein excipient present in an amount of less than 4% (w/v), optionally, between about 2% (w/v) and about 2.5% (w/v), immediately before drying.

In some embodiments, the flavivirus immunogen is selected from the group consisting of a live attenuated flavivirus, an inactivated flavivirus, a chimeric flavivirus, and a recombinant flavivirus immunogen. In some embodiments, the flavivirus is chosen from a yellow fever virus, a Japanese encephalitis virus, a dengue virus, and a Zika virus. In some embodiments, the flavivirus immunogen is present in any amount between 0.001 and 20 standard doses.

In some embodiments, the protein stabilizer is selected from the group consisting of a silk fibroin, an albumin, a gelatin, or a combination thereof.

In some embodiments, the protein stabilizer is present before, e.g., immediately before, drying in an amount from 0.1% (w/v) to 20% (w/v). In some embodiments, the protein stabilizer is present in an amount from 0.5 milligrams to 100 milligrams per standard dose. In some embodiments, the protein stabilizer is present in an amount from 0.001 milligrams to 2 grams.

In some embodiments, the sugar or sugar alcohol excipient is selected from the group consisting of a sucrose, a trehalose, a sorbitol, a mannitol, or a combination thereof.

In some embodiments, the sugar or sugar alcohol excipient is present before, e.g. immediately before, drying in an amount over 1% (w/v) and up to 20% (w/v). In some embodiments, the sugar or sugar alcohol excipient is present in an amount over 5 milligrams and up to 100 milligrams per standard dose. In some embodiments, the sugar or sugar alcohol is present in an amount from 0.005 milligrams to 2 grams.

In some embodiments, the substantially dried flavivirus vaccine preparation is dried by a process selected from the group consisting of air-drying, air-drying with secondary drying, and lyophilization.

In some embodiments, the substantially dried flavivirus vaccine preparation comprises water in an amount between 0% and 5%. In some such embodiments, the preparation is produced by lyophilization.

In some embodiments, the substantially dried flavivirus vaccine preparation comprises water in an amount between 5% and 20%. In some such embodiments, the preparation is produced by air-drying or by air-drying with secondary drying.

In some embodiments, the stabilized liquid flavivirus vaccine preparation retains at least 70% of its original bioactivity after storage at 25° C. for 4 weeks, at least 60% of its original bioactivity after storage at 37° C. for 4 weeks, and/or at least 60% of its original bioactivity after storage at 45° C. for 4 weeks.

In another aspect, the invention provides methods of treating or preventing an infection caused by a flavivirus, comprising the step of administering to a subject in need thereof a therapeutically or prophylactically effective amount of a stabilized liquid or substantially-dried flavivirus vaccine preparation of the invention, thereby eliciting an immune response in the subject and treating or preventing the infection.

In one aspect, the invention provides a method of eliciting an immune response to a virus in a subject. The method includes administering to a subject in need thereof an flavivirus vaccine preparation as described herein in an amount sufficient to elicit the immune response to the virus.

In some embodiments, the subject is selected from a human and a non-human mammal. In some embodiments, the subject is an adult or a child. In some embodiments, the vaccine is administered by a route selected from the group consisting of oral, subcutaneous, dermal (e.g., transdermal, intradermal or interdermal), and intramuscular.

These and other aspects and embodiment of the invention will be apparent to one of ordinary skill in the art from the following detailed description, drawings and examples.

### Rotavirus

The present invention discloses, at least in part, substantially dry rotavirus vaccine preparations with surprisingly increased stability over time and/or at elevated temperatures. In some embodiments, the rotavirus vaccine preparation includes a rotavirus immunogen, a protein excipient (also referred to interchangeably herein as a “protein stabilizer”), and a sugar or sugar alcohol excipient. In some embodiments, the rotavirus vaccine preparation can further comprise a divalent cation. The rotavirus vaccine preparation can be produced by forming a solution of the vaccine antigen with a protein excipient, and substantially drying the resulting solution by a techniques including lyophilization, vacuum-drying, and/or air-drying.

Thus, in certain embodiments, the invention provides a substantially dried, stabilized vaccine formulation comprising a rotavirus immunogen, a protein stabilizer, a sugar excipient, and, optionally, a divalent cation. In certain embodiments, the stabilized vaccine formulation retains significant bioactivity when stored at 37° C. or 45° C. for at least six months. In certain embodiments, the stabilized vaccine formulation retains significant bioactivity when stored at 20° C. or 25° C. for up to two years. In certain embodiments, the rotavirus vaccine preparation has one, two, three, or four of the following properties: (i) retains at least 30%, 40%, or 50% of its original bioactivity after storage at 40-45° C. for 3-6 months, (ii) retains at least 30%, 40%, or 50% of its original bioactivity after storage at 45° C. for 4, 8 or 12 weeks; (iii) retains at least 30%, 40%, 50% or 60% of its original bioactivity after storage at 37° C. for 4, 8 or 12 weeks; or (iv) retains at least 70%, 80% or 90% of its original bioactivity after storage at 25° C. for 4, 8, or 12 weeks, e.g., when (i)-(iv) are tested in the vaccine preparation comprising the protein excipient present in an amount of less than 4% (w/v), optionally, between about 2% (w/v) and about 2.5% (w/v), immediately before drying.

Thus, in one aspect, the invention provides a substantially dried rotavirus vaccine preparation comprising: a rotavirus immunogen; a protein excipient; and a sugar or sugar alcohol excipient. In some embodiments, the rotavirus is selected from a G1, G2, G3, G4 or G9 serotype. In some embodiments, the rotavirus is selected from a P[4], P[6] or P[8] genotype. In some specific embodiments, the rotavirus is P1A[8] human reassortant strain. In some embodiments, the rotavirus immunogen is selected from the group consisting of a live attenuated rotavirus and an inactivated rotavirus. In specific embodiments, the rotavirus is a human rotavirus reassortant strain.

In some embodiments, the rotavirus immunogen is present in any amount between 0.001 and 20 standard doses. In some embodiments, the rotavirus immunogen is one or more of the following: between 2.2×10³ and 4.4×10⁷ infectious units (IU) of a G1 human reassortant strain, between 2.8×10³ and 5.6×10⁷ IU of a G2 human reassortant strain, between 2.2×10³ and 4.4×10⁷ IU of a G3 human reassortant strain, between 2.0×10³ and 4.0×10⁷ IU of a G4 human reassortant strain, and/or between 2.3×10³ and 4.6×10⁷ IU of a type P[8] human reassortant strain. In some embodiments, rotavirus immunogen is an amount between 10³ and 2×10⁷ mean Cell Culture Infectious Dose (CCID₅₀) of a live attenuated rotavirus.

In some embodiments, the rotavirus immunogen is one or more of the following: between 2.2×10³ and 4.4×10⁷ IU of a type G1 strain, between 2.8×10³ and 5.6×10⁷ IU of a type G2 strain, between 2.2×10³ and 4.4×10⁷ IU of a type G3 strain, between 2.0×10³ and 4.0×10⁷ IU of a type G4 strain, between 2.0×10³ and 5.6×10⁷ IU of a type G9 strain, between 2.0×10³ and 5.6×10⁷ IU of a type P[4] strain, between 2.0×10³ and 5.6×10⁷ IU of a type P[6] strain, and/or between 2.3×10³ and 4.6×10⁷ IU of a type P[8] strain.

In some embodiments, the rotavirus immunogen is one or more of the following: between 10³ and 2×10⁷ CCID₅₀ of a type G1 strain, between 10³ and 2×10⁷ CCID₅₀ of a type G2 strain, between 10³ and 2×10⁷ CCID₅₀ of a type G3 strain, between 10³ and 2×10⁷ CCID₅₀ of a type G4 strain, between 10³ and 2×10⁷ CCID₅₀ of a type G9 strain, between 10³ and 2×10⁷ CCID₅₀ of a type P[4] strain, between 10³ and 2×10⁷ CCID₅₀ of a type P[6] strain, and/or between 10³ and 2×10⁷ CCID₅₀ of a type P[8] strain.

In some embodiments, the protein excipient is selected from a silk fibroin, a gelatin and an albumin, or a combination thereof.

In some embodiments, the protein excipient is present before, e.g., immediately before, drying in an amount from 0.01% to 10% (w/v). In some embodiments, the protein excipient is present before, e.g., immediately before, drying in an amount from 0.1% to 10% (w/v). In some embodiments, the protein excipient is present before, e.g., immediately before, drying in an amount from 0.5% to 10% (w/v). In some embodiments, the protein excipient is present before, e.g., immediately before, drying in an amount from 0.5% to 5% (w/v).

In some embodiments, the protein excipient is present in an amount between 2.0 mg and 3.2 g per standard dose of rotavirus immunogen. In some embodiments, the protein excipient is present in an amount between 10 mg and 3.2 g per standard dose of rotavirus immunogen. In some embodiments, the protein excipient is present in an amount between 10 mg and 200 mg per standard dose of rotavirus immunogen. In some embodiments, the protein excipient is present in an amount between 10 mg and 100 mg per standard dose of rotavirus immunogen. In some embodiments, the protein excipient is present in an amount between 160 mg and 3.2 g per standard dose of rotavirus immunogen. In some embodiments, the protein excipient is present in an amount between 160 mg and 1.6 g per standard dose of rotavirus immunogen.

In some embodiments, the protein excipient is present in an amount between 0.002 mg to 64 g. In some embodiments, the protein excipient is present in an amount between 0.01 mg and 64 g. In some embodiments, the protein excipient is present in an amount between 0.01 mg and 4 g. In some embodiments, the protein excipient is present in an amount between 0.01 mg and 2 g. In some embodiments, the protein excipient is present in an amount between 0.16 mg and 64 g. In some embodiments, the protein excipient is present in an amount between 0.16 mg and 32 g.

In some embodiments, the protein excipient is present in an amount between 2.0 mg and 3.2 g. In some embodiments, the protein excipient is present in an amount between 10 mg and 3.2 g. In some embodiments, the protein excipient is present in an amount between 10 mg and 200 mg. In some embodiments, the protein excipient is present in an amount between 10 mg and 100 mg. In some embodiments, the protein excipient is present in an amount between 160 mg and 3.2 g. In some embodiments, the protein excipient is present in an amount between 160 mg and 1.6 g.

In some embodiments, the sugar or sugar alcohol excipient is selected from a sucrose, a trehalose, a sorbitol and a glycerol, or a combination thereof.

In some embodiments, the sugar or sugar alcohol excipient is present before, e.g., immediately before, drying in an amount from 0.1% to 20% (w/v). In some embodiments, the sugar or sugar alcohol excipient is present before, e.g., immediately before, drying in an amount from 0.1% to 15% (w/v). In some embodiments, the sugar or sugar alcohol excipient is present before, e.g., immediately before, drying in an amount from 0.5% to 15% (w/v). In some embodiments, the sugar or sugar alcohol excipient is present before, e.g., immediately before, drying in an amount from 0.5% to 10% (w/v). In some embodiments, the sugar or sugar alcohol excipient is present before, e.g., immediately before, drying in an amount from 1% to 10% (w/v).

In some embodiments, the sugar or sugar alcohol excipient is present in an amount between 2.0 mg to 16 g per standard dose of rotavirus immunogen. In some embodiments, the sugar or sugar alcohol excipient is present in an amount between 32 mg to 16 g per standard dose of rotavirus immunogen. In some embodiments, the sugar or sugar alcohol excipient is present in an amount between 160 mg to 16 g per standard dose of rotavirus immunogen. In some embodiments, the sugar or sugar alcohol excipient is present in an amount between 320 mg to 8 g per standard dose of rotavirus immunogen. In some embodiments, the sugar or sugar alcohol excipient is present in an amount between 320 mg to 3.2 g per standard dose of rotavirus immunogen. In some embodiments, the sugar or sugar alcohol excipient is present in an amount between 2.0 mg to 1 g per standard dose of rotavirus immunogen. In some embodiments, the sugar or sugar alcohol excipient is present in an amount between 10 mg to 1 g per standard dose of rotavirus immunogen. In some embodiments, the sugar or sugar alcohol excipient is present in an amount between 20 mg to 500 mg per standard dose of rotavirus immunogen. In some embodiments, the sugar or sugar alcohol excipient is present in an amount between 20 mg to 200 mg per standard dose of rotavirus immunogen.

In some embodiments, the sugar or sugar alcohol excipient is present in an amount between 0.002 mg to 320 g. In some embodiments, the sugar or sugar alcohol excipient is present in an amount between 0.032 mg to 320 g. In some embodiments, the sugar or sugar alcohol excipient is present in an amount between 0.16 mg to 320 g. In some embodiments, the sugar or sugar alcohol excipient is present in an amount between 0.32 mg to 160 g. In some embodiments, the sugar or sugar alcohol excipient is present in an amount between 0.32 mg to 64 g. In some embodiments, the sugar or sugar alcohol excipient is present in an amount between 0.002 mg to 20 g. In some embodiments, the sugar or sugar alcohol excipient is present in an amount between 0.01 mg to 20 g. In some embodiments, the sugar or sugar alcohol excipient is present in an amount between 0.02 mg to 10 g. In some embodiments, the sugar or sugar alcohol excipient is present in an amount between 0.02 mg to 4 g.

In some embodiments, the divalent cation is selected from the group consisting of Ca²⁺, Mg²⁺, Mn²⁺, and Cu²⁺, or a combination thereof.

In some embodiments, the divalent cation is present before, e.g., immediately before, drying in an amount from 0.1 mM to 1 M. In some embodiments, the divalent cation is present before, e.g., immediately before, drying in an amount from 0.1 mM to 100 mM. In some embodiments, the divalent cation is present before, e.g., immediately before, drying in an amount from 1 mM to 100 mM.

In some embodiments, the divalent cation is present in an amount between 2.0×10⁻⁷ and 3.2×10⁻³ moles per standard dose of rotavirus immunogen. In some embodiments, the divalent cation is present in an amount between 2.0×10⁻⁶ and 3.2×10⁻³ moles per standard dose of rotavirus immunogen. In some embodiments, the divalent cation is present in an amount between 2.0×10⁻⁶ and 2.0×10⁻⁴ moles per standard dose of rotavirus immunogen. In some embodiments, the divalent cation is present in an amount between 3.2×10⁻⁵ and 3.2×10⁻³ moles per standard dose of rotavirus immunogen.

In some embodiments, the divalent cation is present in an amount between 2.0×10⁻¹⁰ to 0.064 moles. In some embodiments, the divalent cation is present in an amount between 2.0×10⁻⁹ and 0.064 moles. In some embodiments, the divalent cation is present in an amount between 2.0×10⁻⁹ and 4.0×10⁻³ moles. In some embodiments, the divalent cation is present in an amount between 3.2×10⁻⁸ and 0.064 moles. In some embodiments, the divalent cation is present in an amount between 2.0×10⁻⁷ and 3.2×10⁻³ moles. In some embodiments, the divalent cation is present in an amount between 2.0×10⁻⁶ and 3.2×10⁻³ moles. In some embodiments, the divalent cation is present in an amount between 2.0×10⁻⁶ and 2.0×10⁻⁴ moles. In some embodiments, the divalent cation is present in an amount between 3.2×10⁻⁵ and 3.2×10⁻³ moles.

In some embodiments, the buffer has buffering capacity between pH 3 and pH 8, between pH 4 and pH 7.5, or between pH 5 and pH 7. In some embodiments, the buffer is selected from the group consisting of HEPES and a CP buffer.

In some embodiments, the buffer is present before, e.g., immediately before, drying in an amount from 0.1 mM to 1 M. In some embodiments, the buffer is present before, e.g., immediately before, drying in an amount from 0.1 mM to 100 mM. In some embodiments, the buffer is present before, e.g., immediately before, drying in an amount from 1 mM to 100 mM.

In some embodiments, the buffer is present in an amount between 2.0×10⁻⁷ and 4.0×10⁻³ moles per standard dose of rotavirus immunogen. In some embodiments, the buffer is present in an amount between 2.0×10⁻⁶ and 4.0×10⁻³ moles per standard dose of rotavirus immunogen. In some embodiments, the buffer is present in an amount between 2.0×10⁻⁶ and 2.0×10⁻⁴ moles per standard dose of rotavirus immunogen. In some embodiments, the buffer is present in an amount between 4.0×10⁻⁵ and 4.0×10⁻³ moles per standard dose of rotavirus immunogen.

In some embodiments, the buffer is present in an amount between 2.0×10⁻¹⁰ to 0.08 moles. In some embodiments, the buffer is present in an amount between 2.0×10⁻⁹ and 0.08 moles. In some embodiments, the buffer is present in an amount between 2.0×10⁻⁹ and 4.0×10⁻³ moles. In some embodiments, the buffer is present in an amount between 4.0×10⁻⁸ and 0.08 moles. In some embodiments, the buffer is present in an amount between 2.0×10⁻⁷ and 4.0×10⁻³ moles. In some embodiments, the buffer is present in an amount between 2.0×10⁻⁶ and 4.0×10⁻³ moles. In some embodiments, the buffer is present in an amount between 2.0×10⁻⁶ and 2.0×10⁻⁴ moles. In some embodiments, the buffer is present in an amount between 4.0×10⁻⁵ and 4.0×10⁻³ moles.

In some embodiments, the preparation is dried by a process selected from the group consisting of air-drying, vacuum drying and lyophilization, or a combination thereof. In some embodiments, the preparation comprises water in an amount between 0% and 5%, and in some of those embodiments, the preparation is produced by lyophilization. In some embodiments, the preparation comprises water in an amount between 5% and 20%, and in some of those embodiments, the preparation is produced by air-drying.

In some embodiments, the preparation retains at least 70%, 80% or 90% of its original bioactivity after storage at 25° C. for 2 weeks; at least 70%, 80% or 90% of its original bioactivity after storage at 25° C. for 4 weeks; at least 70%, 80% or 90% of its original bioactivity after storage at 25° C. for 8 weeks; and/or at least 70%, 80% or 90% of its original bioactivity after storage at 25° C. for 12 weeks.

In some embodiments, the preparation retains at least 60%, 70%, or 80% of its original bioactivity after storage at 37° C. for 2 weeks; at least 60%, 70%, or 80% of its original bioactivity after storage at 37° C. for 4 weeks; at least 50%, 60%, or 70% of its original bioactivity after storage at 37° C. for 8 weeks; and/or at least 30%, 40%, or 50% of its original bioactivity after storage at 37° C. for 12 weeks.

In some embodiments, the preparation retains at least 50%, 60%, or 70% of its original bioactivity after storage at 45° C. for 2 weeks; at least 30%, 40%, or 50% of its original bioactivity after storage at 45° C. for 4 weeks; at least 30%, 40%, or 50% of its original bioactivity after storage at 45° C. for 8 weeks; and/or at least 30%, 40%, or 50% of its original bioactivity after storage at 45° C. for 12 weeks.

In another aspect, the invention provides a method of treating or preventing an infection caused by a rotavirus, by administering to a subject in need thereof a therapeutically or prophylactically effective amount of a vaccine preparation of the invention, thereby eliciting an immune response in the subject and treating or preventing the infection.

In one aspect, the invention provides a method of eliciting an immune response to a virus in a subject. The method includes administering to a subject in need thereof an rotavaccine preparation as described herein in an amount sufficient to elicit the immune response to the virus.

In some embodiments, the subject is selected from a human and a non-human mammal. In some embodiments, the subject is an adult or a child. In some embodiments, the vaccine is administered by a route selected from oral, subcutaneous, transdermal and intramuscular.

In another aspect, the invention provides a method of making a substantially dried vaccine preparation, e.g., a large-scale substantially dried viral vaccine preparation. The method includes:

(i) mixing: (a) a viral immunogen; (b) a protein excipient, e.g., selected from the group consisting of a silk fibroin, a gelatin and an albumin, or a combination thereof; (c) a sugar or a sugar alcohol excipient, e.g., selected from the group consisting of a sucrose, a trehalose, a sorbitol and a glycerol, or a combination thereof; and (d) optionally, a divalent cation, thereby forming a vaccine mixture, and

(ii) lyophilizing or drying, e.g., air drying, the vaccine mixture at about 2° C. to about 50° C. (e.g., at about 20° C. to about 25° C., and e.g., at about 20% to about 40% relative humidity). In some embodiments, a large-scale formulation is prepared at about 1-million dosage units per year.

In one aspect, the invention provides a large-scale substantially dried viral vaccine preparation as described herein. In embodiments, the large-scale vaccine preparation is made according to the methods as described herein.

These and other embodiments of the invention are described in the following figures, detailed description and claims.

### BRIEF DESCRIPTION OF THE DRAWINGS

The following drawings are illustrative of embodiments of the invention and are not meant to limit the scope of the invention as encompassed by the claims.

FIG. 1 depicts the stability of inactivated polio vaccine (IPV) Type 1 in an air-dried film formulation over 26 weeks at 25° C. (▪), 37° C. (♦), and 45° C. (▴) (normalized to 4° C. control), in a vacuum-dried formulation over 8 weeks at 45° C. (∘) (normalized to 4° C. control), and in the commercial IPOL formulation over 4 weeks at 45° C. (●). The films and vacuum-dried samples were made from a pre-drying solution of one-tenth of one standard dose (as defined herein) of trivalent IPV, 2.4% (w/v) silk, 5% (w/v) sucrose, 10 mM magnesium chloride, and 10 mM citrate-phosphate buffer, dried, incubated at the temperatures and for the durations indicated above, and subsequently reconstituted in an aqueous solution of 0.01M PBS (pH 7.2), 0.25% w/v TWEEN® 20 (polysorbate 20), and 0.5% w/v gelatin prior to analysis by D-antigen ELISA.

FIG. 2 depicts the stability of inactivated polio vaccine (IPV) Type 2 in an air-dried film formulation over 26 weeks at 25° C. (▪), 37° C. (♦), and 45′C (▴) (normalized to 4° C. control), in a vacuum-dried formulation over 8 weeks at 45° C. (∘) (normalized to 4′C control), and in the commercial IPOL formulation over 4 weeks at 45° C. (●). The films and vacuum-dried samples were made from a pre-drying solution of one-tenth of one standard dose (as defined herein) of trivalent IPV, 2.4% (w/v) silk, 5% (w/v) sucrose, 10 mM magnesium chloride, and 10 mM citrate-phosphate buffer, dried, incubated at the temperatures and for the durations indicated above, and subsequently reconstituted in an aqueous solution of 0.01M PBS (pH 7.2), 0.25% w/v TWEEN® 20 (polysorbate 20), and 0.5% w/v gelatin prior to analysis by D-antigen ELISA.

FIG. 3 depicts the stability of inactivated polio vaccine (IPV) Type 3 in an air-dried film formulation over 26 weeks at 25° C. (▪), 37° C. (♦), and 45° C. (▴) (normalized to 4° C. control), in a vacuum-dried formulation over 8 weeks at 45° C. (∘) (normalized to 4° C. control), and in the commercial IPOL formulation over 4 weeks at 45° C. (●). The films and vacuum-dried samples were made from a pre-drying solution of one-tenth of one standard dose (as defined herein) of trivalent IPV, 2.4% (w/v) silk, 5% (w/v) sucrose, 10 mM magnesium chloride, and 10 mM citrate-phosphate buffer, dried, incubated at the temperatures and for the durations indicated above, and subsequently reconstituted in an aqueous solution of 0.01M PBS (pH 7.2), 0.25% w/v TWEEN® 20 (polysorbate 20), and 0.5% w/v gelatin prior to analysis by D-antigen ELISA.

FIG. 4 depicts the stability of inactivated polio vaccine (IPV) Type 1 in air-dried film formulations over 56 days at 45° C. and in the commercial IPOL formulation over 26 days at 45° C. (●). The films were made from a pre-drying solution of one-tenth of one standard dose (as defined herein) of trivalent IPV, 2.4% (w/v) protein stabilizer, 2.4% (w/v) sugar excipient, 10 mM magnesium chloride, and 10 mM citrate-phosphate buffer, wherein the protein stabilizer and sugar excipient were bovine serum albumin and sucrose (▪), bovine serum albumin and trehalose (♦), gelatin and sucrose (▴), gelatin and trehalose (□), gelatin and sorbitol (⋄), and silk fibroin and trehalose (Δ), then dried, incubated at the temperatures and for the durations indicated above, and subsequently reconstituted in an aqueous solution of 0.01M PBS (pH 7.2), 0.25% w/v TWEEN® 20 (polysorbate 20), and 0.5% w/v gelatin prior to analysis by D-antigen ELISA.

FIG. 5 depicts the stability of inactivated polio vaccine (IPV) Type 2 in air-dried film formulations over 56 days at 45° C. and in the commercial IPOL formulation over 26 days at 45° C. (●). The films were made from a pre-drying solution of one-tenth of one standard dose (as defined herein) of trivalent IPV, 2.4% (w/v) protein stabilizer, 2.4% (w/v) sugar excipient, 10 mM magnesium chloride, and 10 mM citrate-phosphate buffer, wherein the protein stabilizer and sugar excipient were bovine serum albumin and sucrose (▪), bovine serum albumin and trehalose (♦), gelatin and sucrose (▴), gelatin and trehalose (□), gelatin and sorbitol (⋄), and silk fibroin and trehalose (Δ), then dried, incubated at the temperatures and for the durations indicated above, and subsequently reconstituted in an aqueous solution of 0.01M PBS (pH 7.2), 0.25% w/v TWEEN® 20 (polysorbate 20), and 0.5% w/v gelatin prior to analysis by D-antigen ELISA.

FIG. 6 depicts the stability of inactivated polio vaccine (IPV) Type 3 in air-dried film formulations over 56 days at 45° C. and in the commercial IPOL formulation over 26 days at 45° C. (●). The films were made from a pre-drying solution of one-tenth of one standard dose (as defined herein) of trivalent IPV, 2.4% (w/v) protein stabilizer, 2.4% (w/v) sugar excipient, 10 mM magnesium chloride, and 10 mM citrate-phosphate buffer, wherein the protein stabilizer and sugar excipient were bovine serum albumin and sucrose (▪), bovine serum albumin and trehalose (♦), gelatin and sucrose (▴), gelatin and trehalose (□), gelatin and sorbitol (⋄), and silk fibroin and trehalose (Δ), then dried, incubated at the temperatures and for the durations indicated above, and subsequently reconstituted in an aqueous solution of 0.01M PBS (pH 7.2), 0.25% w/v TWEEN® 20 (polysorbate 20), and 0.5% w/v gelatin prior to analysis by D-antigen ELISA.

FIG. 7 depicts the stability of rotavirus vaccine over 87 days at 45° C. in various lyophilized formulations as compared to a control of RotaTeq® (Merck & Co.) maintained at 4° C. for that same period of time (∘). The samples were made from a pre-drying solution of one-fifth of one standard dose (as defined herein) of rotavirus vaccine combined with either: 10 mM calcium chloride, and 12.6 mM HEPES buffer (▪); 2% (w/v) silk fibroin, 10 mM calcium chloride, and 12.6 mM HEPES buffer (♦); 5% (w/v) sucrose, 10 mM calcium chloride (▴), and 12.6 mM HEPES buffer; or 2% (w/v) silk fibroin, 5% (w/v) sucrose, 10 mM calcium chloride, and 12.6 mM HEPES buffer (●). They were then dried by lyophilization, incubated at 45° C., and subsequently reconstituted prior to analysis by RT-PCR as described in Example 11, specific to the G1 reassortant rotavirus strain.

FIG. 8 depicts the stability of rotavirus vaccine in a lyophilized formulation over 154 days at 4° C. (●), 25° C. (▪), 37° C. (♦), and 45° C. (▴) as compared to controls of RotaTeq® (Merck Co.) maintained at 4° C. (∘) and 45° C. (□) for that same period of time. The lyophilized samples were made from a pre-drying solution of one-fifth one standard dose (as defined herein) of rotavirus vaccine, 2% (w/v) silk, 5% (w/v) sucrose, 10 mM calcium chloride, and 12.6 mM HEPES buffer, dried by lyophilization, incubated at the temperatures and for the durations indicated above, and subsequently reconstituted prior to analysis by RT-PCR as described in Example 11, specific to the G1 reassortant rotavirus strain.

FIG. 9 depicts the stability of rotavirus vaccine over 28 days at 45° C. in various lyophilized formulations as compared to a control of RotaTeq® (Merck & Co.) maintained at 4° C. for that same period of time (∘). The samples were made from a pre-drying solution of one-fifth of one standard dose (as defined herein) of rotavirus vaccine, 2% (w/v) silk fibroin, 5% (w/v) sucrose, 10 mM calcium chloride, and either: 9.76 mM HEPES buffer (●) or 9.76 mM citrate-phosphate buffer (▪). They were then dried by lyophilization, incubated at 45° C., and subsequently reconstituted prior to analysis by RT-PCR, as described in Example 11, specific to the G1 reassortant rotavirus strain.

FIG. 10 depicts the stability of rotavirus vaccine over 56 days at 45° C. in various air-dried formulations as compared to a control of RotaTeq® (Merck & Co.) maintained at 4° C. for that same period of time (∘). The samples were made from a pre-drying solution of one-tenth of one standard dose (as defined herein) of rotavirus vaccine combined with either: 2% (w/v) silk fibroin, 10 mM calcium chloride, and 12.6 mM HEPES buffer (▪); or 2% (w/v) silk fibroin, 5% (w/v) sucrose, 10 mM calcium chloride, and 12.6 mM HEPES buffer (●). They were then air-dried as films, incubated at 45° C., and subsequently reconstituted prior to analysis by RT-PCR, as described in Example 11, specific to the G1 reassortant rotavirus strain.

FIG. 11 depicts the stability of rotavirus vaccine over 165 days at 45° C. in an air-dried formulation (●) as compared to controls of RotaTeq® (Merck & Co.) maintained at 4° C. (∘) and 45° C. (□) for that same period of time. The samples were made from a pre-drying solution of one-fifth of one standard dose (as defined herein) of rotavirus vaccine combined with 2% (w/v) silk fibroin, 5% (w/v) sucrose, 10 mM calcium chloride, and 14.8 mM HEPES buffer. They were then air-dried as films, incubated at 45° C., and subsequently reconstituted prior to analysis by RT-PCR, as described in Example 11, specific to the G1 reassortant rotavirus strain.

FIG. 12 depicts the stability of yellow fever vaccine at 45° C. in (a) an air-dried film made from a pre-drying solution of one-fifth of one standard dose of YF-Vax® reconstituted in water for injection (WFI) with no added excipients, (b) an air-dried (with secondary drying) film made from a pre-drying solution of one-fifth of one standard dose of YF-Vax®, 2.5% (w/v) silk fibroin, and 5% (w/v) sucrose, (c) an air-dried film made from a pre-drying solution of one-fifth of one standard dose of YF-Vax®, 2.5% (w/v) silk fibroin, and 5% (w/v) trehalose, (d) an air-dried film made from a pre-drying solution of one-fifth of one standard dose of YF-Vax® and 5% (w/v) sucrose, and (e) the commercial YF-Vax® lyophilized formulation. After being maintained at 45° C. for the time periods indicated, the formulations were reconstituted in water for injection (WFI) prior to analysis of potency by CCID₅₀.

FIG. 13 depicts the stability of yellow fever vaccine at 45° C. in (a) an air-dried film made from a pre-drying solution of one-fifth of one standard dose of YF-Vax® reconstituted in water for injection (WFI) with no added excipients, (b) an air-dried film made from a pre-drying solution of one-fifth of one standard dose of YF-Vax®, 2.5% (w/v) gelatin, and 5% (w/v) sucrose, (c) an air-dried film made from a pre-drying solution of one-fifth of one standard dose of YF-Vax®, 2.5% (w/v) silk fibroin, and 5% (w/v) sucrose, and (d) an air-dried film made from a pre-drying solution of one-fifth of one standard dose of YF-Vax®, 5% (w/v) silk fibroin, and 5% (w/v) sucrose. After being maintained at 45° C. for the time periods indicated, the formulations were reconstituted in water for injection (WFI) prior to analysis of potency by CCID₅₀.

FIG. 14 depicts the stability of yellow fever vaccine at 45° C. in (a) an air-dried film made from a pre-drying solution of one-fifth of one standard dose of YF-Vax® reconstituted in water for injection (WFI) with no added excipients, (b) an air-dried film made from a pre-drying solution of one-fifth of one standard dose of YF-Vax®, 2.5% (w/v) silk fibroin, and 5% (w/v) sucrose, and buffer in an amount that maintained the pH at 6.2, (c) an air-dried film made from a pre-drying solution of one-fifth of one standard dose of YF-Vax®, 2.5% (w/v) silk fibroin and 5% (w/v) sucrose with no added buffer (pH 6.57); (d) an air-dried film made from a pre-drying solution of one-fifth of one standard dose of YF-Vax®, 2.5% (w/v) silk fibroin, and 5% (w/v) sucrose, and buffer in an amount that maintained the pH at 6.7,; (e) an air-dried film made from a pre-drying solution of one-fifth of one standard dose of YF-Vax®, 2.5% (w/v) silk fibroin, 5% (w/v) sucrose, and HEPES buffer in an amount that maintained the pH at 7.5; and (f) an air-dried film made from a pre-drying solution of one-fifth of one standard dose of YF-Vax®, 2.5% (w/v) silk fibroin, 5% (w/v) sucrose, and HEPES buffer in an amount that maintained the pH at 8.0 After being maintained at 45° C. for the time periods indicated, the formulations were reconstituted in water for injection (WFI) prior to analysis of potency by CCID50 .

FIG. 15A depicts the stability of one standard dose of yellow fever vaccine (YF-Vax®) at 4° C. after reconstitution in: (a) a solution of 0.9% (w/v) NaCl; and (b) a solution of 0.9% (w/v) NaCl and 4% (w/v) silk fibroin. After being maintained at 4° C. for the time periods indicated, potency was analyzed by CCID50.

FIG. 15B depicts the stability of one standard dose of yellow fever vaccine (YF-Vax®) at 25° C. after reconstitution in: (a) a solution of 0.9% (w/v) NaCl; and (b) a solution of 0.9% (w/v) NaCl and 4% (w/v) silk fibroin. After being maintained at 25° C. for the time periods indicated, potency was analyzed by CCID50.

FIG. 15C depicts the stability of one standard dose of yellow fever vaccine (YF-Vax®) at 37° C. after reconstitution in: (a) a solution of 0.9% (w/v) NaCl; and (b) a solution of 0.9% (w/v) NaCl and 4% (w/v) silk fibroin. After being maintained at 37° C. for the time periods indicated, potency was analyzed by CCID50.

FIG. 16 depicts the stability of one standard dose of yellow fever vaccine (YF-Vax®) at 37° C. after reconstitution in (a) a solution of 0.9% (w/v) NaCl; (b) a solution of 0.9% (w/v) NaCl and 0.1% (w/v) silk fibroin; (c) a solution of 0.9% (w/v) NaCl and 1% (w/v) silk fibroin; (d) a solution of 0.9% (w/v) NaCl and 4% (w/v) silk fibroin; and (e) a solution of 0.9% (w/v) NaCl and 7.75% (w/v) silk fibroin. After being maintained at 37° C. for the time periods indicated, potency was analyzed by CCID50.

FIG. 17 depicts the stability of one standard dose of yellow fever vaccine (YF-Vax®) at 37° C. after reconstitution in (a) a solution of 0.9% (w/v) NaCl; (b) a solution of 0.9% (w/v) NaCl and 1% (w/v) silk fibroin; (c) a solution of 0.9% (w/v) NaCl and 4% (w/v) silk fibroin; (d) a solution of 0.9% (w/v) NaCl and 1% (w/v) hydrolyzed silk fibroin; and (e) a solution of 0.9% (w/v) NaCl and 4% (w/v) hydrolyzed silk fibroin. After being maintained at 37° C. for the time periods indicated, potency was analyzed by CCID50.

FIG. 18 depicts the stability of one standard dose of yellow fever vaccine (YF-Vax®) at 37° C. after reconstitution in (a) a solution of 0.9% (w/v) NaCl; (b) a solution of 0.9% (w/v) NaCl and 0.1% (w/v) bovine serum albumin (BSA); (c) a solution of 0.9% (w/v) NaCl and 1% (w/v) bovine serum albumin (BSA); and (d) a solution of 0.9% (w/v) NaCl and 1% (w/v) gelatin. After being maintained at 37° C. for the time periods indicated, potency was analyzed by CCID50.

FIG. 19 depicts the stability of one standard dose of yellow fever vaccine (YF-Vax®) at 37° C. after reconstitution in (a) a solution of 0.9% (w/v) NaCl; (b) a solution of 0.9% (w/v) NaCl, 1% (w/v) silk fibroin, and 0.1% (w/v) BSA; (c) a solution of 0.9% (w/v) NaCl, 1% (w/v) silk fibroin, and 1% (w/v) BSA; (d) a solution of 0.9% (w/v) NaCl, 1% (w/v) silk fibroin, and 1% (w/v) gelatin; (e) a solution of 0.9% (w/v) NaCl, 1% (w/v) gelatin, and 0.1% (w/v) BSA; and (f) a solution of 0.9% (w/v) NaCl, 1% (w/v) silk fibroin, 1% (w/v) gelatin, and 0.1% (w/v) BSA. After being maintained at 37° C. for the time periods indicated, potency was analyzed by CCID50.

FIG. 20 depicts the stability of Japanese encephalitis vaccine at 45° C. in (a) the commercial IMOJEV® lyophilized formulation and (b) an air-dried film made from a pre-drying solution of one-tenth of one standard dose of IMOJEV® and 4% (w/v) silk. After being maintained at 45° C. for the time periods indicated, the formulations were reconstituted in water for injection (WFI) prior to analysis of potency by CCID₅₀.

### DETAILED DESCRIPTION

### Overview

The present invention depends, in part, upon the discovery that substantially dry viral vaccine (e.g., enterovirus, rotavirus, and flavivirus vaccine) preparations with surprisingly increased stability over time and/or at elevated temperatures can be produced by forming solutions of the vaccine antigen with certain protein stabilizers and/or sugar stabilizers, and substantially drying the resulting solution by techniques including lyophilization, vacuum-drying, and air-drying.

In certain embodiments, the invention provides a substantially dried, stabilized vaccine formulation comprising an enterovirus antigen, such as IPV or an inactivated coxsackie virus or rhinovirus, a protein stabilizer, a sugar excipient, and a divalent cation.

In certain embodiments, the invention provides a substantially dried, stabilized vaccine formulation comprising a rotavirus antigen, a protein stabilizer, a sugar excipient, and a divalent cation.

In certain embodiments, the stabilized vaccine formulations comprising the enterovirus antigen or the rotavirus antigen retain significant bioactivity when stored at 37° C. or 45° C. for at least six months. In certain embodiments, the stabilized vaccine formulations retain significant bioactivity when stored at 20° C. or 25° C. for up to two years.

In certain embodiments, the invention provides a substantially dried stabilized vaccine formulation comprising a flavivirus antigen, a protein stabilizer, such as silk fibroin, gelatin, albumin, or a combination thereof, and a sugar or sugar alcohol, such as sucrose, trehalose, sorbitol, mannitol, or a combination thereof. In certain embodiments, the invention provides a substantially dried stabilized vaccine formulation comprising a flavivirus antigen, a protein stabilizer chosen from silk fibroin, gelatin, albumin, or a combination thereof, and a sugar or sugar alcohol chosen from sucrose, trehalose, sorbitol, mannitol, or a combination thereof. In certain embodiments, the substantially dried stabilized vaccine formulation is lyophilized. In certain embodiments, the substantially dried stabilized vaccine formulation is air-dried. In certain embodiments, the substantially dried stabilized vaccine formulation is air-dried with secondary drying. In certain embodiments, the substantially dried stabilized vaccine formulation comprising the flavivirus antigen retains significant bioactivity when stored at 45° C. for up to two months. In certain embodiments, the substantially dried stabilized vaccine formulation retains significant bioactivity when stored at approximately 25° C. for up to two years.

In other embodiments, the invention provides a liquid stabilized vaccine formulation comprising a flavivirus antigen and a protein stabilizer chosen from silk fibroin, albumin, gelatin, or a combination thereof. In certain embodiments, the invention provides a liquid stabilized vaccine formulation comprising a flavivirus antigen and a protein stabilizer chosen from silk fibroin, albumin, or a combination thereof. In certain embodiments, the liquid stabilized vaccine formulation retains significant bioactivity when stored at 4° C. for up to 5 weeks. In certain embodiments, the liquid stabilized vaccine formulation retains significant bioactivity when stored at 25° C. for up to 72 hours. In certain embodiments, the liquid stabilized vaccine formulation retains significant bioactivity when stored at 37° C. for up to 12 hours.

### Definitions

All scientific and technical terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent or later-developed techniques which would be apparent to one of skill in the art. In addition, in order to more clearly and concisely describe the subject matter which is the invention, the following definitions are provided for certain terms which are used in the specification and appended claims.

The articles “a” and “an” are used herein to refer to one or to more than one (i.e., to at least one) of the grammatical object of the article. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

As used herein, an “adjuvant” is a substance that is able to favor or amplify the cascade of imirnunological events, ultimately leading to a better (e.g., increased) immunological response, i.e., the integrated bodily response to an antigen, including cellular and/or humoral responses. An adjuvant is in general not required for the immunological response to occur, but favors or amplifies this response.

As used herein, the term “antigen” refers to a molecule or a portion of a molecule capable of being bound by a selective binding agent, such as an antibody, and/or capable of being recognized by the immune system, and/or capable of inducing a humoral immune response and/or cellular immune response leading to the activation of B and/or T lymphocytes. An antigen may have one or more epitopes. Antigens as used herein may also be mixtures of several individual antigens.

As used herein, the term “dose” means the amount of an antigen or immunogen which is administered (e.g., in a vaccination) to elicit an immune response (e.g., humoral or cellular immunity) in an organism.

As used herein, a “standard dose” means the amount of antigen in a typical human dose of a vaccine, as approved for marketing by national or international regulatory authorities (e.g., U.S. FDA, EMEA).

With respect to Salk IPV, this is equivalent to 40 D-antigen units in the case of inactivated Type 1 poliovirus antigen, 8 D-antigen units in the case of inactivated Type 2 poliovirus antigen, 32 D-antigen unit in the case of inactivated Type 3 poliovirus antigens, or any combination of one or more of the foregoing in the case of a monovalent, bivalent, or trivalent IPV vaccine. With respect to Sabin IPV, this is equivalent to up to 40 D-antigen units in the case of inactivated Type 1 poliovirus antigen, up to 50 D-antigen units in the case of inactivated Type 2 poliovirus antigen, up to 64 D-antigen unit in the case of inactivated Type 3 poliovirus antigens, or any combination of one or more of the foregoing in the case of a monovalent, bivalent, or trivalent IPV vaccine.

With respect to certain live reassortant rotavirus vaccines (e.g., RotaTeq®), this is equivalent to at least 2.2×10⁶ IU of a G1 human reassortant strain, at least 2.8×10⁶ IU of a G2 human reassortant strain, at least 2.2×10⁶ IU of a G3 human reassortant strain, at least 2.0×10⁶ IU of a G4 human reassortant strain, and at least 2.3×10⁶ of a P1A[8] human reassortant strain, or any combination of one or more of the foregoing in the case of a monovalent or multivalent rotavirus vaccine. With respect to certain live attenuated rotavirus vaccines (e.g., Rotarix®), this is equivalent to at least 10⁶ median cell culture infective dose (CCID₅₀) of live, attenuated rotavirus.

With respect to live attenuated yellow fever vaccine, this is equivalent to not less than 4.74 log₁₀ plaque forming units (PFU) per 0.5 mL dose. With respect to live attenuated recombinant Japanese encephalitis vaccine, this is equivalent to between 4.0 and 5.8 log₁₀ PFU per 0.5 mL dose. With respect to live attenuated recombinant dengue vaccine, this is equivalent to between 4.5 and 6.0 log₁₀ 50% cell culture infective dose (CCID₅₀) of each serotype of the virus included in the vaccine per 0.5 mL dose.

As used herein, the term “bioactivity” of a vaccine preparation (or of the antigenic or immunogenic components of the vaccine preparation), refers to the ability of the vaccine preparation (or its antigenic or immunogenic components) to elicit the desired immune response. As a proxy for determining bioactivity of a live and/or attenuated virus vaccine, the titer of live virus can be measured. As a proxy for determining bioactivity of a killed pathogen and/or non-live virus vaccine (e.g., an inactivated viral vaccine such as IPV or a subunit viral vaccine), the quantity of a correctly folded antigen can be measured (e.g., using a conformation-specific antibody against the antigen). Alternatively, direct measures of immunogenicity can be measured, such as the ability to elicit humoral or cellular immune responses. In some embodiments, when referring to a formulation that retains a certain a percentage of bioactivity after storage under certain conditions, that can be measured, for example, by dividing the titer (as measured by, e.g., log₁₀ CCID₅₀/mL) of the formulation after such storage by the titer of the formulation before such storage.

As used herein, the term “enterovirus” refers to a virus wiihin the enterovirus genus of positive-sense single-stranded RNA viruses within the picornavirus family. An enterovirus can be a live, wild-type virus, a live attenuated virus, an inactivated virus, a chimeric virus, or a viral vector or viral subunit comprising a peptide or protein derived from an enterovirus capsid or genome. Examples of enteroviruses include, but are not limited to, the polio viruses, coxsackie viruses, rhinoviruses and echo viruses.

As used herein, the term “rotavirus” refers to a virus within the rotavirus genus of double-stranded RNA viruses within the Reoviridae family. A rotavirus can be a live wild-type virus, a live attenuated virus, an inactivated virus, a reassortant or chimeric virus, or a viral vector or viral subunit comprising a peptide or protein derived from an rotavirus capsid or genome.

As used herein, the term “flavivirus” refers to a virus within the flavivirus genus of positive-sense single-stranded RNA viruses within the Flaviviridae family. A flavivirus can be a live wild-type virus, a live attenuated virus, an inactivated virus, a chimeric virus, or a

recombinant virus. Examples of flaviviruses include, but are not limited to, yellow fever virus, Japanese encephalitis virus, dengue virus, and Zika virus.

As used herein, the term “measles virus” refers to a virus within the morbillivirus genus of single-stranded, negative-sense, enveloped (non-segmented) RNA viruses within the Paramyxovirus family. A measles virus can be a live wild-type virus, a live attenuated virus, an inactivated virus, a chimeric virus, or a recombinant virus.

As used herein, the term “mumps virus” refers to a virus within the rubulavirus genus of linear, single-stranded, negative-sense RNA viruses within the Paramyxoviridae family. A mumps virus can be a live wild-type virus, a live attenuated virus, an inactivated virus, a chimeric virus, or a recombinant virus.

As used herein, the term “rubella virus” refers to a virus within the rubivirus genus of single-stranded, positive-sense RNA viruses within the Togaviridae family. A rubella virus can be a live wild-type virus, a live attenuated virus, an inactivated virus, a chimeric virus, or a recombinant virus.

As used herein, the term “influenza virus” refers to a negative-sense ssRNA virus within the Orthomyxoviridae family. An influenza virus can be a live wild-type virus, a live attenuated virus, an inactivated virus, a chimeric virus, or a recombinant virus. Examples of influenza viruses include influenza A, influenza B, and influenza. C.

As used herein, the term “immunogen” refers to any substance (e.g., an antigen, combination of antigens, pathogen fragment, whole pathogen) capable of eliciting an immune response in an organism. An “immunogen” is capable of inducing an immunological response against itself after administration to a mammalian subject. The term “immunological” as used herein with respect to an immunological response, refers to the development of a humoral (antibody mediated) and/or a cellular (mediated by antigen-specific T cells or their secretion products) response directed against an immunogen in a recipient subject. Such a response can be an active response induced by administration of an immunogen or immunogenic peptide to a subject or a passive response induced by administration of antibody or primed T cells that are directed towards the immunogen. In some embodiments, an immunogen is an enterovirus, a flavivirus, a rotavirus, a measles virus, a mumps virus, a rubella virus, or an influenza virus, or a fragment thereof. In some embodiments, an inactivated or live attenuated polio virus, or antigenic fragment thereof, is an immunogen. In some embodiments, an inactivated or live attenuated rotavirus, or antigenic fragment thereof, is an immunogen. In some embodiments, an inactivated, live attenuated or recombinant flavivirus, or antigenic fragment thereof, is an immunogen.

As used herein, the term “immunogenicity” refers to the ability of a substance, such as an antigen or epitope, to provoke humoral and/or cell-mediated immunological response in a subject. A skilled artisan can readily measure immunogenicity of a substance. The presence of a cell-mediated immunological response can be determined by any art-recognized methods, e.g., proliferation assays (CD4+ T cells), CTL (cytotoxic T lymphocyte) assays, or immunohistochemistry with tissue section of a subject to determine the presence of activated cells such as monocytes and macrophages after the administration of an immunogen. One of skill in the art can readily determine the presence of humoral-mediated immunological response in a subject by any well-established methods. For example, the level of antibodies produced in a biological sample such as blood can be measured by western blot, ELISA or other methods known for antibody detection.

As used herein, the term “infectivity” in reference to a virus means the efficacy of a virus at infecting the cells of a susceptible host and reproducing therein. Any methods known to a skilled artisan for determination of virus infectivity can be used for the purposes described herein.

As used herein, the term “killed pathogens” is used in reference to pathogens that were previously virulent (i.e., able to cause disease) but have been destroyed or rendered non-infective or non-virulent with chemicals or heat. Inactivated polio vaccine is an example of a vaccine comprising a killed pathogen.

As used herein, the term “live attenuated pathogens” refers to pathogens that have not been inactivated, i.e., pathogens capable of replicating in permissive cells and inducing a specific immunological response, but do not induce the disease or infectious state caused by the corresponding wild-type pathogens in a subject. Live attenuated pathogens can be produced by one of skill in the art, e.g., by cultivating wild-type pathogens under conditions that disable, reduce, and/or eliminate their virulent properties, or using closely-related but less virulent organisms to produce such an immunological response. An example of the use of a live attenuated pathogen in a vaccine is yellow fever vaccine or live attenuated rotavirus vaccine. An example of the use of a live attenuated pathogen in a vaccine is. The term “live attenuated pathogens” encompasses live attenuated reassortant or chimeric viruses, such as live reassortant rotavirus vaccine. An example of the use of a live attenuated pathogen in a vaccine is live attenuated yellow fever vaccine. The term “live attenuated pathogens” encompasses live attenuated chimeric or live attenuated recombinant viruses.

As used herein, when referring to the bioactivity of the vaccine preparations of the invention, the term “retain” means to keep, sustain, or maintain a specified or significant percentage of the original bioactivity of at least one antigen in the preparation with respect to the time at which the preparation was prepared.

As used herein, the term “a monovalent vaccine” refers to a vaccine that is designed to immunize against a single antigen or single microorganism.

As used herein, the term “multivalent or polyvalent vaccine” refers to a vaccine that is designed to immunize against two or more antigens, two or more different strains of a microorganism, or against two or more different microorganisms. For example, a divalent vaccine is generally a vaccine that is designed to immunize against two different antigens, two different strains of a microorganism or against two different microorganisms. A trivalent vaccine is generally a vaccine that is designed to immunize against three different antigens, three different strains of a microorganism or against three different microorganisms. An exemplary trivalent vaccine is a vaccine that is designed to immunize against measles, mumps, and rubella. An exemplary multivalent vaccine is a vaccine that is designed to immunize against multiple strains of rotavirus.

As used herein, the term “pathogen” means any disease-producing agent (especially a virus or bacterium or other microorganism).

As used herein, the term “potency” means, with respect to IPV, the D-antigen content of the vaccine for any one of poliovirus Types 1, 2 or 3. A vaccine preparation that produces a precipitin line at the distance of 25 mm from the center is defined as having a value of 600 D-antigen units (see, e.g., Edens et al. (2015), Vaccine 33:4683-4690). As used herein, the term “potency” is synonymous with the “bioactivity” for IPV.

As used herein, the term “potency” means: with respect to live reassortant rotavirus vaccine or live attenuated rotavirus vaccine, the titer of a vaccine preparation, whether measured by infectious units (IU), CCID₅₀, or other methods known in the art; or with respect to a non-live virus vaccine (e.g., an inactivated or subunit viral vaccine), the quantity of antigen (e.g., using a conformation-specific antibody against the antigen) present in the preparation. As used herein, the term “potency” is synonymous with “bioactivity” for rotavirus.

As used herein, the term “potency” means, with respect to live attenuated yellow fever or live attenuated recombinant Japanese encephalitis vaccine, the number of plaque forming units (PFU) in said vaccine, and with respect to live attenuated recombinant dengue vaccine, the titer of the vaccine as measured by 50% cell culture infective dose (CCID₅₀).

The term “pre-determined amount” is generally used in reference to an amount of a formulation desired and/or determined by a user, e.g., depending on applications or treatment. In some embodiments, the term “pre-determined amount” refers to an amount of a formulation effective to treat or prevent a disease or a disorder, e.g., increasing immunity to the disease; reducing, inhibiting or delaying at least one symptom of the disease; or producing an improvement in the disease, for example, beneficial or desired clinical results. For the purposes of various aspects described herein, beneficial or desired clinical results include, but are not limited to, alleviation of one or more symptoms, diminishment of extent of disease, stabilized (e.g., not worsening) state of disease, delay or slowing of disease progression, amelioration or palliation of the disease state, and remission (whether partial or total), whether detectable or undetectable. In some embodiments, treating can refer to prolonging survival as compared to expected survival if not receiving treatment. Thus, one of skill in the art realizes that a treatment may improve the disease condition, but may not be a complete cure for the disease. In reference to immunogenic or vaccine formulation, the term “pre-determined amount” can mean an amount of the formulation effective to provide or increase immunity to a particular disease. A blood test or any methods known to a skilled artisan can be used to check immunity. Accordingly, in some embodiments, the delivery device comprises an effective dose of immunogenic or vaccine formulation.

As used herein, the term “silk fibroin” includes silkworm fibroin and insect or spider silk protein. Any type of silk fibroin can be used according to various aspects described herein. Silk fibroin produced by silkworms, such as Bombyx mori, is the most common and represents an earth-friendly, renewable resource. For instance, silk fibroin used in a silk film may be obtained by extracting sericin from the cocoons of B. mori. Organic silkworm cocoons are also commercially available. There are many different silks, however, including spider silk (e.g., obtained from Nephila clavipes), transgenic silks, genetically engineered silks, such as silks from bacteria, yeast, mammalian cells, transgenic animals, or transgenic plants (see, e.g., WO 97/08315; U.S. Pat. No. 5,245,012), and variants thereof, that can be used.

As used herein, the term “gelatin” means a sterile nonpyrogenic protein preparation (e.g., fractions) produced by partial acid hydrolysis (type A gelatin) or by partial alkaline hydrolysis (type B gelatin) of animal collagen, most commonly derived from cattle, pig, and fish sources. Gelatin can be obtained in varying molecular weight ranges. Recombinant sources of gelatin may also be used.

As used herein, the term “albumin” includes a sterile nonpyrogenic preparation of serum albumin, most commonly obtained from healthy human donors or derived from bovine sources. Albumin from egg may also be present in some vaccine formulations as a result of the viral production process. Recombinant sources of albumin may also be used.

As used herein, the terms “stabilizing,” “stabilize,” “stability,” and “stabilization,” refer to retaining the bioactivity of at least one antigen in a vaccine preparation, such that, for example, one or more antigens in a formulation retain at least about 30% of its original bioactivity, at least about 40%, at least about 50%, at least about 60%, at least about 70%, at least about 80%, or at least about 90% of its original bioactivity.

As used herein, a “subject” means a human or animal. Usually the animal is a vertebrate such as a primate, rodent, domestic animal or game animal. Primates include chimpanzees, cynomologous monkeys, spider monkeys, and macaques (e.g., Rhesus). Rodents include mice, rats, woodchucks, ferrets, rabbits and hamsters. Domestic and game animals include cows, horses, pigs, deer, bison, buffalo, feline species (e.g., domestic cat), canine species (e.g., dog, fox, wolf), avian species (e.g., chicken, emu, ostrich), and fish (e.g., trout, catfish and salmon). In certain embodiments of the aspects described herein, the subject is a mammal (e.g., a primate, e.g., a human). A subject can be male or female. In certain embodiments, the subject is a mammal. The mammal can be a human, non-human primate, mouse, rat, dog, cat, horse, or cow, but are not limited to these examples. In addition, the methods and formulations described herein can be used to treat domesticated animals and/or pets.

As used herein, a “substantially dry” formulation or preparation of a vaccine means a composition in which there is 20% (w/w) or less residual moisture content (RMC). A substantially dry formulation or preparation may, in some cases, be prepared by substantially removing the water from a vaccine that has been formulated in a solution or liquid mixture. The removal of the liquid can be accomplished by various means (e.g., by passive evaporation, by evaporation assisted by vacuum or other conditions, and/or by sublimation such as by lyophilization (freeze-drying)). The substantially dry formulations can be reconstituted in a pharmaceutically acceptable carrier prior to administration, In particular embodiments, the vaccine formulations of the invention are substantially dried formulations comprising 5% to 20% (w/w), or at least 4.6% (w/w) (e.g., 4% to 10%), e.g., residual moisture content. In some particular embodiments, the vaccine formulations of the invention are substantially dried formulations comprising 0.5% to 5% (w/w) residual moisture content.

The term “vaccine” as used herein refers to any preparation of an antigen (including subunit antigens, toxoid antigens, conjugate antigens, or other types of antigenic molecules) or a killed or live attenuated microorganism that, when introduced into a subject's body, affects the immune response to the specific antigen or microorganism by causing activation of the immune system against the specific antigen or microorganism (e.g., inducing antibody formation, T cell responses, and/or B-cell responses). Generally, vaccines against microorganisms are directed toward at least part of a virus, bacteria, parasite, mycoplasma, or other infectious agent.

As used herein, the term “viruses” refers to an infectious agent composed of a nucleic acid encapsidated in a protein. Such infectious agents are incapable of autonomous replication (i.e., replication requires the use of the host cell's machinery). Viral genomes can be single-stranded (ss) or double-stranded (ds), RNA or DNA, and can or cannot use reverse transcriptase (RT). Additionally, ssRNA viruses can be either sense (+) or antisense (−). Exemplary viruses include, but are not limited to, dsDNA viruses (e.g., Adenoviruses, Herpesviruses, Poxviruses), ssDNA viruses (e.g., Parvoviruses), dsRNA viruses (e.g., Reo viruses), (+)ssRNA viruses (e.g., Picomaviruses, Toga viruses), (−)ssRNA viruses (e.g., Orthomyxoviruses, Rhabdoviruses), ssRNA-RT viruses, i.e., (+)sense RNA with DNA intermediate in life-cycle (e.g., Retroviruses), and dsDNA-RT viruses (e.g., Hepadnaviruses). In some embodiments, viruses can also include wild-type (natural) viruses, killed viruses, live attenuated viruses, modified viruses, recombinant viruses or any combinations thereof. Other examples of viruses include, but are not limited to, enveloped viruses, respiratory syncytial viruses, non-enveloped viruses, bacteriophages, recombinant viruses, and viral vectors. The term “bacteriophages” as used herein refers to viruses that infect bacteria.

The patent, scientific and technical literature referred to herein establish knowledge that was available to those skilled in the art at the time of filing. The entire disclosures of the issued U.S. patents, published and pending patent applications, and other publications that are cited herein are hereby incorporated by reference to the same extent as if each was specifically and individually indicated to be incorporated by reference. In the case of any inconsistencies, the present disclosure will prevail.

### Exemplary Enterovirus Vaccine Formulations

Overview

While there is no cure for poliomyelitis, vaccination with inactivated poliovirus vaccine (IPV) and live attenuated oral polio vaccine (OPV) has eliminated the disease in much of the world. In the absence of effective vaccination, nearly 1 in 200 children worldwide would be expected to acquire paralytic poliomyelitis (Sutter et al. (2008), Indian Pediatr. 45(5):353-5). Through the efforts of the Global Polio Eradication Initiative, the largest public health initiative in history, only three countries (Afghanistan, Nigeria, and Pakistan) remained polio-endemic as of 2012. In parallel with continued efforts toward eradication of polio, the global community has recognized the need to prepare for post-eradication immunization. While OPV has played an important role in decreasing wild-type poliovirus cases, the live attenuated vaccine can lead to rare cases of polio, either in recipients or their close contacts (vaccine-associated paralytic polio) or through viruses that have circulated and mutated, developing neurovirulence and transmissibility properties of wild polio viruses (circulating vaccine-derived polioviruses). This necessitates cessation of OPV and a switch to IPV within 3 years of wild-type poliovirus interruption to eradicate the disease and maintain immunity. Post-eradication demand for IPV could be as high as 425 million doses annually (Venczel et al., “Global Post-Eradication IPV Supply and Demand Assessment: Integrated Findings,” Oliver Wyman, Inc., 2009). Furthermore, the World Health Organization has expressed interest in the development of an inactivated polio vaccine that contains inactivated versions of the non-infectious Sabin virus strains used in OPV for greater safety in the case of release of live virus from a production facility. Removing IPV from the constraints of the cold chain would make a significant contribution to the global effort to eradicate polio by reducing costs and simplifying logistics related to cold storage and vaccine spoilage.

In certain embodiments, the invention relates to a substantially dried (e.g., lyophilized, vacuum-dried, or air-dried) vaccine formulation comprising, consisting essentially of, or consisting of an antigen, a protein stabilizer, a sugar or a sugar alcohol excipient, a divalent cation, and a buffer salt. In some embodiments, the protein stabilizer is selected from silk fibroin, gelatin, and albumin. In some embodiments, the sugar or the sugar alcohol excipient is selected from sucrose, trehalose, sorbitol, and glycerol, or combinations thereof. In some embodiments, the divalent cation is selected from Ca²⁺, Mg²⁺, Mn²⁺, and Cu²⁺. In some embodiments, the buffer salt is selected from HEPES and citrate phosphate (CP).

In certain embodiments, the invention relates to a substantially dried vaccine formulation comprising, consisting essentially of, or consisting of an enterovirus immunogen, a protein, a sugar or a sugar alcohol, a divalent cation salt, and a buffer salt. In some embodiments, the protein is selected from silk fibroin, gelatin and albumin. In some embodiments, the sugar or the sugar alcohol is selected from sucrose, trehalose, sorbitol, and glycerol, or combinations thereof. In some embodiments, the divalent cation salt is magnesium chloride. In some embodiments, the buffer salt is HEPES or CP.

In certain embodiments, the invention relates to a substantially dried vaccine formulation comprising, consisting essentially of, or consisting of an enterovirus immunogen, a protein, a sugar or a sugar alcohol, a divalent cation salt, and a buffer salt, wherein the protein is selected from silk fibroin, gelatin, and albumin; the sugar or the sugar alcohol is selected from sucrose, trehalose, sorbitol, and glycerol, or combinations thereof; the divalent cation salt is magnesium chloride; and the buffer salt is HEPES or CP.

In certain embodiments, the invention relates to a substantially dried vaccine formulation comprising, consisting essentially of, or consisting of an enterovirus immunogen, a protein, a sugar or a sugar alcohol, a divalent cation salt, and a buffer salt, wherein the protein is selected from silk fibroin, gelatin, and albumin; the sugar or the sugar alcohol is sucrose; and the buffer salt is HEPES or CP.

In certain embodiments, the invention relates to a substantially dried vaccine formulation comprising, consisting essentially of, or consisting of an enterovirus immunogen, a protein, a sugar or a sugar alcohol, a divalent cation salt, and a buffer salt, wherein the protein is selected from silk fibroin, gelatin, and albumin; the sugar or the sugar alcohol is sucrose; the divalent cation salt is magnesium chloride; and the buffer salt is HEPES or CP.

In certain embodiments, the invention relates to a substantially dried vaccine formulation comprising, consisting essentially of, or consisting of an enterovirus immunogen, a protein, a sugar or a sugar alcohol, a divalent cation salt, and a buffer salt, wherein the enterovirus is poliovirus; the protein is selected from silk fibroin, gelatin, and albumin; the sugar or the sugar alcohol is selected from sucrose, trehalose, sorbitol, and glycerol, or combinations thereof; and the buffer salt is HEPES or CP.

In certain embodiments, the invention relates to a substantially dried vaccine formulation comprising, consisting essentially of, or consisting of an enterovirus immunogen, a protein, a sugar or a sugar alcohol, a divalent cation salt, and a buffer salt, wherein the enterovirus is poliovirus; the protein is selected from silk fibroin, gelatin, and albumin; the sugar or the sugar alcohol is selected from sucrose, trehalose, sorbitol, and glycerol, or combinations thereof; the divalent cation salt is magnesium chloride; and the buffer salt is HEPES or CP.

In certain embodiments, the invention relates to a substantially dried vaccine formulation comprising, consisting essentially of, or consisting of an enterovirus immunogen, a protein, a sugar or a sugar alcohol, a divalent cation salt, and a buffer salt, wherein the enterovirus is poliovirus; the protein is selected from silk fibroin, gelatin, and albumin; the sugar or the sugar alcohol is sucrose; and the buffer salt is HEPES or CP.

In certain embodiments, the invention relates to a substantially dried vaccine formulation comprising, consisting essentially of, or consisting of an enterovirus immunogen, a protein, a sugar or a sugar alcohol, a divalent cation salt, and a buffer salt, wherein the enterovirus is poliovirus; the protein is selected from silk fibroin, gelatin, and albumin; the sugar or the sugar alcohol is sucrose; the divalent cation salt is magnesium chloride; and the buffer salt is HEPES or CP.

In certain embodiments, the invention relates to a substantially dried vaccine formulation comprising, consisting essentially of, or consisting of an enterovirus immunogen, a protein, a sugar or a sugar alcohol, a divalent cation salt, and a buffer salt, wherein the enterovirus is inactivated poliovirus; the protein is selected from silk fibroin, gelatin, and albumin; the sugar or the sugar alcohol is selected from sucrose, trehalose, sorbitol, and glycerol, or combinations thereof; and the buffer salt is HEPES or CP.

In certain embodiments, the invention relates to a substantially dried vaccine formulation comprising, consisting essentially of, or consisting of an enterovirus immunogen, a protein, a sugar or a sugar alcohol, a divalent cation salt, and a buffer salt, wherein the enterovirus is inactivated poliovirus; the protein is selected from silk fibroin, gelatin, and albumin; the sugar or the sugar alcohol is selected from sucrose, trehalose, sorbitol, and glycerol, or combinations thereof; the divalent cation salt is magnesium chloride; and the buffer salt is HEPES or CP.

In certain embodiments, the invention relates to a substantially dried vaccine formulation comprising, consisting essentially of, or consisting of an enterovirus immunogen, a protein, a sugar or a sugar alcohol, a divalent cation salt, and a buffer salt, wherein the enterovirus is inactivated poliovirus; the protein is selected from silk fibroin, gelatin, and albumin; the sugar or the sugar alcohol is sucrose; and the buffer salt is HEPES or CP.

In certain embodiments, the invention relates to a substantially dried vaccine formulation comprising, consisting essentially of, or consisting of an enterovirus immunogen, a protein, a sugar or a sugar alcohol, a divalent cation salt, and a buffer salt, wherein the enterovirus is inactivated poliovirus; the protein is selected from silk fibroin, gelatin, and albumin; the sugar or the sugar alcohol is sucrose; the divalent cation salt is magnesium chloride; and the buffer salt is HEPES or CP.

Enterovirus Immunogens

In certain embodiments, the invention relates to any one of the formulations described herein, wherein the enterovirus immunogen is one or more of the several species of enterovirus, including polio virus, coxsackie virus, human rhinovirus and echo virus, or antigenic fragments thereof.

In certain embodiments, the invention relates to any one of the formulations described herein, wherein the inactivated enterovirus is one or more of the several strains of inactivated poliovirus, including inactivated PV-1, PV-2 or PV-3.

In certain embodiments, the enterovirus is inactivated poliovirus (IPV). IPV is produced from wild-type poliovirus strains of one or more serotypes that have been inactivated (killed) with formalin. As an injectable vaccine, it can be administered alone or in combination with other vaccines (e.g., diphtheria, tetanus, pertussis, hepatitis B, and haemophilus influenza). Generally, three spaced doses are administered to generate adequate levels of seroconversion, and in most countries, a booster dose is provided during late childhood. IPV has been used successfully in the polio eradication programs in a few countries, notably in Scandinavia and the Netherlands, but until recently most countries have used the oral polio vaccine (OPV). IPV provides serum immunity to all three types of poliovirus, resulting in protection against paralytic poliomyelitis. Most studies indicate that the degree of mucosal immunity in the intestine is significantly less than that provided by OPV, although this difference may be less pronounced in the pharyngeal mucosal lining. Adverse events following administration of IPV are very mild and transient. Due to the risks associated with the large quantities of poliovirus needed for IPV production, following the global cessation of poliovirus transmission, high level (BSL-3/polio) containment of all manufacturing and quality control areas where live virus is handled must be implemented.

In certain embodiments, the invention relates to any one of the formulations described herein, wherein the formulation comprises IPOL® (Poliovirus Vaccine Inactivated, produced by Sanofi Pasteur SA) or an equivalent thereof. IPOL is a sterile suspension of three types of poliovirus: Type 1 (Mahoney), Type 2 (MEF-1), and Type 3 (Saukett). IPOL vaccine is a highly purified, inactivated poliovirus vaccine with enhanced bioactivity. Each of the three strains of poliovirus is individually grown in vero cells, a continuous line of monkey kidney cells cultivated on microcarriers. The cells are grown in Eagle MEM modified medium, supplemented with newborn calf bovine serum tested for adventitious agents prior to use, originated from countries free of bovine spongiform encephalopathy. For viral growth, the culture medium is replaced by M-199, without calf bovine serum. This culture technique and improvements in purification, concentration, and standardization of poliovirus antigen produce a more potent and consistent immunogenic vaccine than the inactivated poliovirus vaccine (IPV) available in the US prior to 1988.

Each dose (0.5 mL) of IPOL trivalent vaccine is formulated to contain 40 D-antigen units of Type 1, 8 D-antigen units of Type 2, and 32 D-antigen units of Type 3 poliovirus. For each lot of IPOL vaccine, D-antigen content is determined in vitro using the D-antigen ELISA assay. IPOL vaccine is produced from vaccine concentrates diluted with M-199 medium. Also present are 0.5% of 2-phenoxyethanol and a maximum of 0.02% of formaldehyde per dose as preservatives. Neomycin, streptomycin, and polymyxin B are used in vaccine production; and, although purification procedures eliminate measurable amounts, less than 5 ng neomycin, 200 ng streptomycin, and 25 ng polymyxin B per dose may still be present. The residual calf bovine serum albumin is less than 50 ng/dose in the final vaccine.

In certain embodiments, the invention relates to any one of the formulations described herein, wherein the inactivated virus is present in the formulation in an amount of between about 0.001 and about 20 standard doses (as defined herein). In certain embodiments, the invention relates to any one of the formulations described herein, wherein inactivated Type 1 poliovirus is present in the formulation in an amount of between about 0.04 and 800 D-antigen units, inactivated Type 2 poliovirus is present in the formulation in an amount of between about 0.008 and 1000 D-antigen units, and inactivated Type 3 poliovirus is present in the formulation in an amount of between about 0.032 and 1280 D-antigen units.

Although some formulations will be prepared for a single use to vaccinate a single individual, other formulations comprising many standard doses may be prepared for repeated vaccinations of a single individual, or single (or repeated) vaccinations of multiple individuals (e.g., groups of individuals at a school or in a village).

Any enterovirus vaccine products approved by national or regional regulatory authorities (e.g., U.S. FDA or EMEA) for treating or preventing an enterovirus infection can be included in the formulations described herein.

Protein Stabilizers for Enterovirus Vaccines

The vaccine preparations of the invention include at least one protein stabilizer which aids in retaining the bioactivity of the vaccine antigens. In some embodiments, the protein stabilizer is selected from the group consisting silk fibroin, gelatin and albumin.

In certain embodiments, the invention relates to any one of the formulations described herein, wherein the amount of protein chosen from silk fibroin, gelatin, and albumin present in the formulation immediately before drying is from 0.1% to 10% (w/v). In certain embodiments, the invention relates to any one of the formulations described herein, wherein the amount of protein chosen from silk fibroin, gelatin, and albumin in the formulation is from about 1.0 milligrams to about 100 milligrams per standard dose. In certain embodiments, the invention relates to any one of the formulations described herein, wherein the amount of protein chosen from silk fibroin, gelatin, and albumin in the formulation is from about 0.001 milligrams to about 2 grams.

Hydrolyzed gelatin (Gelita VacciPro®, Sioux City, Iowa) was prepared at 10% (w/v) by dissolving dry mass in reduced volume of water at 60° C. and adding water to achieve desired concentration. The solution was then sterile-filtered (0.2 μm) prior to formulation.

Bovine serum albumin (Sigma-Aldrich, St. Louis, Mo.; product #A3294) was prepared at 10% (w/v) by dissolving dry mass in reduced volume of water and adding water to achieve desired concentration. The solution was then sterile-filtered (0.2 μm) prior to formulation.

Sugar and Sugar Alcohol Excipients

The vaccine preparations of the invention include at least one sugar or sugar alcohol excipient. In some embodiments, the sugar or sugar alcohol is selected from the group consisting of sucrose, trehalose, or sorbitol.

In certain embodiments, the invention relates to any of the formulations described herein, wherein the amount of sugar chosen from sucrose, trehalose, or sorbitol present in the formulation immediately before drying is from 0.1% to 50% (w/v). In certain embodiments, the invention relates to any of the formulations described herein, wherein the amount of sugar chosen from sucrose, trehalose, or sorbitol in the formulation is from about 1.0 milligrams to about 500 milligrams per standard dose. In certain embodiments, the invention relates to any of the formulations described herein, wherein the amount of sugar chosen from sucrose, trehalose, or sorbitol in the formulation is from about 0.001 milligrams to about 10 grams.

Divalent Cations for Enterovirus Vaccines

The vaccine preparations of the invention include at least one divalent cation. In some embodiments, the divalent cation is selected from the group consisting of Ca²⁺, Mg²⁺, Mn²⁺, and Cu²⁺. These divalent cations are conveniently provided by including simple salts of the cations in the preparation. For example, chloride, carbonate or bicarbonate salts can conveniently be used (e.g., CaCl₂, CaCO₃, Ca(HCO₃)₂).

In certain embodiments, the invention relates to any of the formulations described herein, wherein the amount of divalent cationic salt present in the formulation immediately before drying is from 0.1 mM to 100 mM. In certain embodiments, the invention relates to any of the formulations described herein, wherein the amount of divalent cationic salt is from about 10⁻⁷ moles to about 10⁻⁴ moles per standard dose. In certain embodiments, the invention relates to any of the formulations described herein, wherein the amount of divalent cationic salt is from about 10⁻¹⁰ moles to about 2×10⁻³ moles.

Buffers for Enterovirus Vaccines

In certain embodiments, the invention relates to any of the formulations described herein, wherein the amount of buffer present in the formulation immediately before drying is from 0.1 mM to 100 mM. In some embodiments, the invention relates to any of the formulations described herein, wherein the amount of buffer is from about 10⁻⁷ moles to about 10⁻⁴ moles per standard dose. In certain embodiments, the invention relates to any of the formulations described herein, wherein the amount of buffer is from about 10⁻¹⁰ moles to about 2×10⁻³ moles.

In some embodiments, the buffer has buffering capacity between pH 3 and pH 8, or between pH 4 and pH 7.5, or between pH 5 and pH 7. In certain embodiments, the invention relates to any of the formulations described herein, wherein the buffer solution is HEPES or a citrate phosphate (CP) buffer comprising citric acid and sodium phosphate dibasic dehydrate (e.g., McIlvane buffer), preferably at a pH of about 7.

Drying and Water Content for Enterovirus Vaccines

In certain embodiments, the invention relates to any one of the formulations described herein, wherein the formulation is an air-dried formulation. In certain embodiments, the invention relates to any one of the formulations described herein, wherein the formulation has been air-dried at a temperature of from about 2° C. to about 50° C. In certain embodiments, the invention relates to any one of the formulations described herein, wherein the formulation has been air-dried at a temperature of about 5° C., about 10° C., about 15° C., about 20° C., about 25° C., about 30° C., about 35° C., about 40° C., or about 45° C. In certain embodiments, the invention relates to any one of the formulations described herein, wherein the formulation has been air-dried at a temperature of about 23° C.

In certain embodiments, the invention relates to any one of the formulations described herein, wherein the formulation is vacuum-dried. Such vacuum drying can be conducted over an extended period of time (e.g., 6-12 hours) at reduced pressures (e.g., 25-100 mTorr) at varying temperatures (e.g., −10° C. to 40° C.), with lower pressures and higher temperatures reducing drying time. See Example 4.

In certain embodiments, the invention relates to any one of the formulations described herein, wherein the formulation is in the form of a lyophilized powder. For example, in some specific embodiments, the formulation is lyophilized by (1) freezing at −50° C. and holding for 1 hour or more, followed by (2) sublimation (primary drying) at −45 to −35° C. for ˜3 hours to several days under vacuum (˜45-50 microbar), and (3) desorption (secondary drying) at 25-30° C. for ˜3 hours to several days under vacuum (˜10-50 microbar). Those of skill in the art can adjust drying pressures and temperatures for best results or mere convenience.

In certain embodiments, the invention relates to any one of the formulations described herein, wherein the formulation is in the form of a film, for example, an air-dried film.

In certain embodiments, the invention relates to any one of the formulations described herein, wherein the formulation comprises 0% to 5% by mass water. These formulations with low water content (i.e., less than 5%) are most typically produced by lyophilization, but can be produced by vacuum-drying or air-drying. In certain embodiments, the invention relates to any one of the formulations described herein, wherein the formulation comprises water in an amount less than 5% by mass. In certain embodiments, the invention relates to any one of the formulations described herein, wherein the formulation comprises water in an amount less than 4% by mass. In certain embodiments, the invention relates to any one of the formulations described herein, wherein the formulation comprises water in an amount less than 3% by mass. In certain embodiments, the invention relates to any one of the formulations described herein, wherein the formulation comprises water in an amount less than 2% by mass. In certain embodiments, the invention relates to any one of the formulations described herein, wherein the formulation comprises water in an amount less than 1% by mass. In certain embodiments, the invention relates to any one of the formulations described herein, wherein the formulation comprises water in an amount less than 0.5% by mass.

In certain embodiments, the invention relates to any one of the formulations described herein, wherein the formulation comprises water in an amount between 5% and 20%. These formulations with higher water content (i.e., 5%-20%) are preferably produced by air-drying, but can be produced by vacuum-drying or partial lyophilization. Thus, in certain embodiments, the formulations comprise greater than 5%, greater than 6%, greater than 7%, greater than 8%, greater than 9%, greater than 10%, greater than 11%, greater than 12%, greater than 13%, greater than 14%, greater than 15%, greater than 16%, greater than 17%, greater than 18%, or greater than 19%, but in each case less than 20% by mass.

Stability and Bioactivity for Enterovirus Vaccines

In certain embodiments, the invention relates to any one of the formulations described herein, wherein the formulation retains at least about 70% of its original bioactivity after storage at about 25° C. for about 2 weeks. In certain embodiments, the invention relates to any one of the formulations described herein, wherein the formulation retains at least about 80% of its original bioactivity after storage at about 25° C. for about 2 weeks. In certain embodiments, the invention relates to any one of the formulations described herein, wherein the formulation retains at least about 90% of its original bioactivity after storage at about 25° C. for about 2 weeks.

In certain embodiments, the invention relates to any one of the formulations described herein, wherein the formulation retains at least about 70% of its original bioactivity after storage at about 25° C. for about 4 weeks. In certain embodiments, the invention relates to any one of the formulations described herein, wherein the formulation retains at least about 80% of its original bioactivity after storage at about 25° C. for about 4 weeks. In certain embodiments, the invention relates to any one of the formulations described herein, wherein the formulation retains at least about 90% of its original bioactivity after storage at about 25° C. for about 4 weeks.

In certain embodiments, the invention relates to any one of the formulations described herein, wherein the formulation retains at least about 70% of its original bioactivity after storage at about 25° C. for about 8 weeks. In certain embodiments, the invention relates to any one of the formulations described herein, wherein the formulation retains at least about 80% of its original bioactivity after storage at about 25° C. for about 8 weeks. In certain embodiments, the invention relates to any one of the formulations described herein, wherein the formulation retains at least about 90% of its original bioactivity after storage at about 25° C. for about 8 weeks.

In certain embodiments, the invention relates to any one of the formulations described herein, wherein the formulation retains at least about 70% of its original bioactivity after storage at about 25° C. for about 12 weeks. In certain embodiments, the invention relates to any one of the formulations described herein, wherein the formulation retains at least about 80% of its original bioactivity after storage at about 25° C. for about 12 weeks. In certain embodiments, the invention relates to any one of the formulations described herein, wherein the formulation retains at least about 90% of its original bioactivity after storage at about 25° C. for about 12 weeks.

In certain embodiments, the invention relates to any one of the formulations described herein, wherein the formulation retains at least about 60% of its original bioactivity after storage at about 37° C. for about 2 weeks. In certain embodiments, the invention relates to any one of the formulations described herein, wherein the formulation retains at least about 70% of its original bioactivity after storage at about 37° C. for about 2 weeks. In certain embodiments, the invention relates to any one of the formulations described herein, wherein the formulation retains at least about 80% of its original bioactivity after storage at about 37° C. for about 2 weeks.

In certain embodiments, the invention relates to any one of the formulations described herein, wherein the formulation retains at least about 60% of its original bioactivity after storage at about 37° C. for about 4 weeks. In certain embodiments, the invention relates to any one of the formulations described herein, wherein the formulation retains at least about 70% of its original bioactivity after storage at about 37° C. for about 4 weeks. In certain embodiments, the invention relates to any one of the formulations described herein, wherein the formulation maintains at least about 80% of its original bioactivity after storage at about 37° C. for about 4 weeks.

In certain embodiments, the invention relates to any one of the formulations described herein, wherein the formulation retains at least about 50% of its original bioactivity after storage at about 37° C. for about 8 weeks. In certain embodiments, the invention relates to any one of the formulations described herein, wherein the formulation retains at least about 60% of its original bioactivity after storage at about 37° C. for about 8 weeks. In certain embodiments, the invention relates to any one of the formulations described herein, wherein the formulation maintains at least about 70% of its original bioactivity after storage at about 37° C. for about 8 weeks.

In certain embodiments, the invention relates to any one of the formulations described herein, wherein the formulation maintains at least about 30% of its original bioactivity after storage at about 37° C. for about 12 weeks. In certain embodiments, the invention relates to any one of the formulations described herein, wherein the formulation retains at least about 40% of its original bioactivity after storage at about 37° C. for about 12 weeks. In certain embodiments, the invention relates to any one of the formulations described herein, wherein the formulation retains at least about 50% of its original bioactivity after storage at about 37° C. for about 12 weeks.

In certain embodiments, the invention relates to any one of the formulations described herein, wherein the formulation retains at least about 50% of its original bioactivity after storage at about 45° C. for about 2 weeks. In certain embodiments, the invention relates to any one of the formulations described herein, wherein the formulation retains at least about 60% of its original bioactivity after storage at about 45° C. for about 2 weeks. In certain embodiments, the invention relates to any one of the formulations described herein, wherein the formulation retains at least about 70% of its original bioactivity after storage at about 45° C. for about 2 weeks.

In certain embodiments, the invention relates to any one of the formulations described herein, wherein the formulation retains at least about 30% of its original bioactivity after storage at about 45° C. for about 4 weeks. In certain embodiments, the invention relates to any one of the formulations described herein, wherein the formulation retains at least about 40% of its original bioactivity after storage at about 45° C. for about 4 weeks. In certain embodiments, the invention relates to any one of the formulations described herein, wherein the formulation maintains at least about 50% of its original bioactivity after storage at about 45° C. for about 4 weeks.

In certain embodiments, the invention relates to any one of the formulations described herein, wherein the formulation retains at least about 30% of its original bioactivity after storage at about 45° C. for about 8 weeks. In certain embodiments, the invention relates to any one of the formulations described herein, wherein the formulation retains at least about 40% of its original bioactivity after storage at about 45° C. for about 8 weeks. In certain embodiments, the invention relates to any one of the formulations described herein, wherein the formulation retains at least about 50% of its original bioactivity after storage at about 45° C. for about 8 weeks.

In certain embodiments, the invention relates to any one of the formulations described herein, wherein the formulation retains at least about 30% of its original bioactivity after storage at about 45° C. for about 12 weeks. In certain embodiments, the invention relates to any one of the formulations described herein, wherein the formulation retains at least about 40% of its original bioactivity after storage at about 45° C. for about 12 weeks. In certain embodiments, the invention relates to any one of the formulations described herein, wherein the formulation retains at least about 50% of its original bioactivity after storage at about 45° C. for about 12 weeks.

Reconstitution and Administration of Enterovirus Vaccines

In some embodiments, the formulations described herein can be reconstituted in a pharmaceutically acceptable carrier for oral or parenteral administration (e.g., subcutaneous or intramuscular injection). As used herein, the term “pharmaceutically acceptable carrier” refers to any and all solvents, diluents, excipients, dispersion media and the like, which can be used to reconstitute a liquid dosage form. Pharmaceutically acceptable carriers useful in the invention include, but are not limited to, (x) glycols, such as propylene glycol; (xi) polyols, such as glycerin, sorbitol, mannitol and polyethylene glycol (PEG); (xii) esters, such as ethyl oleate and ethyl laurate; (xiii) agar; (xiv) buffering agents, such as magnesium hydroxide and aluminum hydroxide; (xv) alginic acid; (xvi) pyrogen-free water; (xvii) isotonic saline; (xviii) Ringer's solution; (xix) ethyl alcohol; (xx) pH buffered solutions; and oils, such as peanut oil, cottonseed oil, safflower oil, sesame oil, olive oil, corn oil and soybean oil, and other non-toxic compatible substances employed in pharmaceutical formulations.

When administering parenterally, a formulation described herein can be generally reconstituted in a unit dosage injectable form (solution, suspension, emulsion). The formulations suitable for injection include sterile aqueous solutions or dispersions. The carrier can be a solvent or dispersing medium containing, for example, water, cell culture medium, buffers (e.g., phosphate buffered saline (PBS)), polyol (for example, glycerol, propylene glycol, liquid polyethylene glycol, and the like), suitable mixtures thereof. In some embodiments, the pharmaceutical carrier can be a buffered solution (e.g., PBS).

The formulations can also contain auxiliary substances such as wetting or emulsifying agents, pH buffering agents, gelling or viscosity enhancing additives, preservatives, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation. With respect to formulations described herein, however, any vehicle, diluent, or additive used should have to be biocompatible with the antigens described herein. Those skilled in the art will recognize that the components of the formulations should be selected to be biocompatible with respect to the antigen. This will present no problem to those skilled in chemical and pharmaceutical principles, or problems can be readily avoided by reference to standard texts or by simple experiments (not involving undue experimentation).

In certain embodiments, the invention relates to a substantially dried vaccine formulation comprising, consisting essentially of, or consisting of an enterovirus, protein, a sugar or a sugar alcohol, a divalent cation salt, a buffer salt, 2-phenoxyethanol, formaldehyde, neomycin, streptomycin, and polymyxin B, wherein the protein is selected from silk fibroin, gelatin, and albumin; the sugar or the sugar alcohol is selected from sucrose, trehalose, sorbitol, and glycerol, or combinations thereof; and the buffer salt is HEPES or CP.

In certain embodiments, the invention relates to a substantially dried vaccine formulation comprising, consisting essentially of, or consisting of an inactivated poliovirus, a protein, a sugar or a sugar alcohol, a divalent cation salt, a buffer salt, 2-phenoxyethanol, formaldehyde, neomycin, streptomycin, and polymyxin B, wherein the protein is selected from silk fibroin, gelatin, and albumin; the sugar or the sugar alcohol is selected from sucrose, trehalose, sorbitol, and glycerol, or combinations thereof; and the buffer salt is HEPES or CP.

In certain embodiments, the invention relates to a substantially dried vaccine formulation comprising, consisting essentially of, or consisting of an enterovirus, a protein, a sugar or sugar alcohol, magnesium chloride, CP, 2-phenoxyethanol, formaldehyde, neomycin, streptomycin, and polymyxin B, wherein the protein is selected from silk fibroin, gelatin, and albumin; and the sugar or sugar alcohol is selected from sucrose, trehalose, and sorbitol.

In certain embodiments, the invention relates to a substantially dried vaccine formulation comprising, consisting essentially of, or consisting of an inactivated poliovirus, a protein, a sugar or sugar alcohol, magnesium chloride, CP, 2-phenoxyethanol, formaldehyde, neomycin, streptomycin, and polymyxin B, wherein the protein is selected from silk fibroin, gelatin, and albumin; and the sugar or sugar alcohol is selected from sucrose, trehalose, and sorbitol.

Exemplary Methods for Preparing Formulations of Enterovirus Vaccines

In some embodiments, the invention relates to a method of preparing any one of the formulations described herein, comprising the steps of:

mixing; and

lyophilizing or drying the vaccine mixture, thereby forming a substantially dried vaccine mixture.

In some embodiments, the invention relates to any one of the methods described herein, wherein the vaccine mixture is lyophilized. In some embodiments, the invention relates to any one of the methods described herein, wherein the vaccine mixture is lyophilized to form a substantially dried vaccine mixture in the form of a powder.

In some embodiments, the invention relates to any one of the methods described herein, wherein the vaccine mixture is substantially dried, for example, air-dried. In some embodiments, the invention relates to any one of the methods described herein, wherein the vaccine mixture is air-dried to form a substantially dried vaccine mixture in the form of a film.

In some embodiments, the invention relates to any one of the methods described herein, further comprising the step of:

mixing the substantially dried vaccine mixture with a diluent.

In some embodiments, the invention relates to any one of the methods described herein, wherein the solution consists essentially of silk fibroin and water. In some embodiments, the invention relates to any one of the methods described herein, wherein the silk fibroin solution does not comprise sericin. In some embodiments, the invention relates to any one of the methods described herein, wherein the silk fibroin solution does not comprise a salt.

In some embodiments, the invention relates to any one of the methods described herein, further comprising the step of:

preparing the silk fibroin solution from a sample comprising a cocoon from a silkworm Bombyx mori.

The aqueous silk fibroin solution can be prepared using techniques known in the art.

Suitable processes for preparing silk fibroin solutions are disclosed, for example, in U.S. Pat. No. 7,635,755; WO 2005/012606; and WO 2008/127401.

In accordance with the conventional practice, the formulations described herein are desirably processed under aseptic conditions using components which preliminarily have been rendered bacterially sterile. Sterility on storage can be maintained by incorporation of an antigen-compatible germicidal substance such as thimerosal.

### Exemplary Methods of Using Formulations of Enterovirus Vaccines

In certain embodiments, the invention relates to a method of treating or preventing an infection caused by an enterovirus, comprising the step of:

administering to a subject in need thereof a therapeutically or prophylactically effective amount or dose of any one of the formulations described herein, thereby eliciting an immune response in the subject and treating or preventing the infection.

In certain embodiments, the invention relates to any one of the methods described herein, wherein the subject is a mammal.

In certain embodiments, the invention relates to any one of the methods described herein, wherein the subject is a mammal susceptible to or suffering from an infection caused by an enterovirus.

In certain embodiments, the invention relates to any one of the methods described herein, wherein the subject is a human.

In certain embodiments, the invention relates to any one of the methods described herein, wherein the subject is a human under the age of five.

In some embodiments, the invention relates to any one of the methods described herein, wherein the formulation is administered to the subject in two, three, or four spaced doses. In some embodiments, the invention relates to any one of the methods described herein, wherein the formulation is administered to the subject in three spaced doses. For example, the first dose is administered when the subject is from about 6 weeks to about 2 months of age, the second dose is administered when the subject is about 4 months of age, and the third dose is administered when the subject is from about 6 to about 18 months of age. In some embodiments, the invention relates to any one of the methods described herein, wherein an optional fourth spaced dose is administered when the subject is form about 4 to about 6 years of age.

In certain embodiments, the invention relates to any one of the methods described herein, wherein the formulation is in the form of a film.

In certain embodiments, the invention relates to any one of the methods described herein, wherein the formulation is in the form of a powder.

In some embodiments, the invention relates to any one of the methods described herein, wherein the formulation is administered to the subject orally.

In certain embodiments, the invention relates to any one of the methods described herein, wherein the formulation is in the form of a film, further comprising the step of: mixing the formulation with a diluent prior to administering to the subject.

In certain embodiments, the invention relates to any one of the methods described herein, wherein the formulation is in the form of a powder, further comprising the step of: mixing the formulation with a diluent prior to administering to the subject.

In some embodiments, the invention relates to any one of the methods described herein, wherein the formulation is administered to the subject by injection, such as subcutaneous, dermal (e.g., transdermal, intradermal or interdermal), or intramuscular injection.

### Exemplary Flavivirus Vaccine Formulations

Overview

Almost all current lyophilized vaccine products, including flavivirus vaccines such as yellow fever and Japanese encephalitis vaccines, are administered within a short time, such as within one to six or one to eight hours, after reconstitution. If the vaccine is not used within that time, this can lead to significant vaccine wastage, such as in the case of a multi-dose vaccine product. If such a product is not used entirely before the end of the post-reconstitution administration window, the remaining vaccine is typically discarded, leading to increased costs for immunization campaigns and other vaccination efforts. Thus, the need exists for improved flavivirus vaccines as described herein.

In certain embodiments, the invention provides a liquid stabilized vaccine formulation comprising a flavivirus antigen and a protein stabilizer chosen from silk fibroin, albumin, gelatin, or a combination thereof. In certain embodiments, the invention provides a liquid stabilized vaccine formulation comprising a flavivirus antigen and a protein stabilizer chosen from silk fibroin, albumin, or a combination thereof. In certain embodiments, the invention provides a liquid stabilized vaccine formulation comprising a yellow fever antigen and a protein stabilizer chosen from silk fibroin, albumin, or a combination thereof. In certain embodiments, the liquid stabilized vaccine formulation retains significant bioactivity when stored at 4° C. for up to five weeks. In certain embodiments, the liquid stabilized vaccine formulation retains significant bioactivity when stored at 25° C. for up to 72 hours. In certain embodiments, the liquid stabilized vaccine formulation retains significant bioactivity when stored at 37° C. for up to 12 hours.

In certain embodiments, the invention provides a substantially dried stabilized vaccine formulation comprising a flavivirus antigen, a protein stabilizer, such as silk fibroin, gelatin, albumin, or a combination thereof, and a sugar or sugar alcohol, such as sucrose, sorbitol, mannitol, or a combination thereof. In certain embodiments, the invention provides a substantially dried stabilized vaccine formulation comprising a flavivirus antigen, a protein stabilizer chosen from silk fibroin, gelatin, albumin, or a combination thereof, and a sugar or sugar alcohol chosen from sucrose, sorbitol, mannitol, or a combination thereof. In certain embodiments, the substantially dried stabilized vaccine formulation is lyophilized. In certain embodiments, the substantially dried stabilized vaccine formulation is air-dried. In certain embodiments, the substantially dried stabilized vaccine formulation is air-dried with secondary drying. In certain embodiments, the substantially dried stabilized vaccine formulation retains significant bioactivity when stored at 45° C. for up to two months. In certain embodiments, the substantially dried stabilized vaccine formulation retains significant bioactivity when stored at approximately 25° C. for up to two years.

Flavivirus Immunogens

In certain embodiments, the invention relates to any one of the formulations described herein, wherein the flavivirus immunogen is or is derived from one or more of the several species of flavivirus, including yellow fever virus, Japanese encephalitis virus, dengue virus, and Zika virus, or antigenic fragments thereof.

In certain embodiments, the invention relates to any one of the formulations described herein, wherein the flavivirus is a live attenuated yellow fever virus. Live attenuated yellow fever vaccine is produced from wild-type yellow fever strains of one or more serotypes that have been attenuated, e.g. by culturing in chicken embryos. A single dose of live attenuated yellow fever vaccine is generally adequate to provide long-lasting protection to most healthy individuals, but an additional dose may be administered to individuals who may not have had an adequate or sustained immune response or who may continue to be at risk for exposure to yellow fever virus.

In certain embodiments, the invention relates to any one of the formulations described herein, wherein the formulation comprises YF-VAX® (Yellow Fever Vaccine, produced by Sanofi Pasteur SA, Lyon, France) or an equivalent thereof. YF-VAX® contains live attenuated yellow fever virus prepared by culturing the 17D-204 strain of yellow fever virus in living avian leucosis virus-free (ALV-free) chicken embryos. Each dose (0.5 mL) of YF-VAX® vaccine is formulated to contain not less than 4.74 log₁₀ plaque forming units (PFU) of live attenuated yellow fever virus. YF-VAX® also contains sorbitol (<7.5 mg) and gelatin (<7.5 mg) as additional stabilizers, but it contains no preservative. YF-VAX® is lyophilized, hermetically sealed under nitrogen, and is supplied with a separate vial of sterile diluent containing Sodium

Chloride Injection USP. See, e.g., YF-VAX® product insert and references cited therein, including Monath et al. (2002), Am. J. Trop. Med. Hyg 66(5);533-41.

In certain embodiments, the invention relates to any one of the formulations described herein, wherein the flavivirus is a live attenuated recombinant Japanese encephalitis virus. Live attenuated recombinant Japanese encephalitis vaccine is produced by incorporating antigenic proteins from a live attenuated Japanese encephalitis virus with a different live attenuated viral vector.

In certain embodiments, the invention relates to any one of the formulations described herein, wherein the formulation comprises IMOJEV® (Japanese Encephalitis Vaccine, produced by Sanofi Pasteur SA, Lyon, France) or an equivalent thereof. IMOJEV® contains live attenuated recombinant Japanese encephalitis virus prepared by culturing chimeric virus incorporating certain structural premembrane (prM) and envelope (E) proteins from the live-attenuated Japanese encephalitis virus strain SA14-14-2 and the non-structural protein backbone of the live-attenuated yellow fever virus strain 17D in Vero cells. Each dose (0.5 mL) of IMOJEV® vaccine is formulated to contain between 4.0 and 5.8 log₁₀ plaque forming units (PFU) of live attenuated recombinant Japanese encephalitis virus. IMOJEV® also contains mannitol, lactose monohydrate, glutamic acid, potassium hydroxide, histidine, and human serum albumin as additional excipients, but it contains no adjuvant or preservative. IMOJEV® is lyophilized and is supplied with a separate vial of diluent containing 0.9% sodium chloride solution. See, e.g., IMOJEV ® product insert; and Torresi et al. (2010), Vaccine 28(50):7993-8000.

In certain embodiments, the invention relates to any one of the formulations described herein, wherein the flavivirus is a live attenuated recombinant dengue virus. Live attenuated recombinant dengue vaccine is produced by incorporating antigenic proteins from a live attenuated dengue virus with a different live attenuated viral vector.

In certain embodiments, the invention relates to any one of the formulations described herein, wherein the formulation comprises Dengvaxia® (Dengue Tetravalent Vaccine, produced by Sanofi Pasteur SA, Lyon, France) or an equivalent thereof. Dengvaxia® contains four live attenuated recombinant dengue viruses representing each of the four dengue virus serotypes (1, 2, 3, and 4). Each recombinant dengue virus is prepared by culturing chimeric virus incorporating certain structural premembrane (prM) and envelope (E) proteins from wild-type viruses of each of the four dengue serotypes and the non-structural protein backbone of the live-attenuated yellow fever virus strain 17D in Vero cells. Each dose (0.5 mL) of Dengvaxia® vaccine is formulated to contain between 4.5 and 6.0 log₁₀ plaque forming units (PFU) of each of the four live attenuated recombinant dengue virus serotypes. Dengvaxia® also contains L-phenylalanine, L-arginine hydrochloride, sucrose, D-trehalose dehydrate, D-sorbitol, trometamol, and urea as additional excipients, but it contains no adjuvant or preservative. Dengvaxia® is lyophilized and is supplied with a separate vial of diluent containing 0.4% (single-dose presentation) or 0.9% (five-dose presentation) sodium chloride solution. See, e.g., Dengvaxia® product insert; and Gailhardou et al. (2016), PLoS Negl Trop Dis. 10(7):e0004821.

In certain embodiments, the invention relates to any one of the formulations described herein, wherein the flavivirus antigen is present in the formulation in an amount of between about 0.001 and about 20 standard doses (as defined herein). In certain embodiments, the invention relates to any one of the formulations described herein, wherein live attenuated yellow fever virus is present in the formulation in an amount of between about 4.74×10⁻³ log₁₀ PFU and 94.8 log₁₀ PFU. In certain embodiments, the invention relates to any one of the formulations described herein, wherein live attenuated yellow fever virus is present in the formulation in an amount of between about 4.0×10⁻³ log₁₀ PFU and 116 log₁₀ PFU. In certain embodiments, the invention relates to any one of the formulations described herein, wherein live attenuated yellow fever virus is present in the formulation in an amount of between about 4.5×10⁻³ log₁₀ PFU and 120 log₁₀ PFU.

Although some formulations will be prepared for a single use to vaccinate a single individual, other formulations comprising many standard doses may be prepared for repeated vaccinations of a single individual, or single (or repeated) vaccinations of multiple individuals (e.g., groups of individuals at a school or in a village).

Any flavivirus vaccine products approved by national or regional regulatory authorities (e.g., U.S. FDA or EMEA) for treating or preventing a flavivirus infection can be included in the formulations described herein.

Liquid Formulations of Flavivirus Vaccines

In certain embodiments, the invention provides a liquid stabilized vaccine formulation comprising a flavivirus antigen and a protein stabilizer chosen from silk fibroin, albumin, gelatin, or a combination thereof. In certain embodiments, the invention provides a liquid stabilized vaccine formulation comprising a flavivirus antigen and a protein stabilizer chosen from silk fibroin, albumin, or a combination thereof. In certain embodiments, the invention provides a liquid stabilized vaccine formulation comprising a yellow fever antigen and a protein stabilizer chosen from silk fibroin, albumin, or a combination thereof. In certain embodiments, the liquid stabilized vaccine formulation retains significant bioactivity when stored at 4° C. for up to five weeks. In certain embodiments, the liquid stabilized vaccine formulation retains significant bioactivity when stored at 25° C. for up to 72 hours. In certain embodiments, the liquid stabilized vaccine formulation retains significant bioactivity when stored at 37° C. for up to 12 hours.

Substantially Dried Formulations of Flavivirus Vaccines

In certain embodiments, the invention provides a substantially dried stabilized vaccine formulation comprising a flavivirus antigen, a protein stabilizer, such as silk fibroin, gelatin, albumin, or a combination thereof, and a sugar or sugar alcohol, such as sucrose, trehalose, sorbitol, mannitol, or a combination thereof. In certain embodiments, the invention provides a substantially dried stabilized vaccine formulation comprising a flavivirus antigen, a protein stabilizer chosen from silk fibroin, gelatin, albumin, or a combination thereof, and a sugar or sugar alcohol chosen from sucrose, trehalose, sorbitol, mannitol, or a combination thereof. In certain embodiments, the invention provides a substantially dried stabilized vaccine formulation comprising a flavivirus antigen, a protein stabilizer chosen from silk fibroin and gelatin, and a sugar excipient chosen from sucrose, trehalose, and mannitol. In certain embodiments, the substantially dried stabilized vaccine formulation is lyophilized. In certain embodiments, the substantially dried stabilized vaccine formulation is air-dried. In certain embodiments, the substantially dried stabilized vaccine formulation is air-dried with secondary drying. In certain embodiments, the substantially dried stabilized vaccine formulation retains significant bioactivity when stored at 45° C. for up to two months. In certain embodiments, the substantially dried stabilized vaccine formulation retains significant bioactivity when stored at approximately 25° C. for up to two years.

In certain embodiments, the invention provides a substantially dried stabilized vaccine formulation comprising a flavivirus antigen chosen from yellow fever virus, Japanese encephalitis virus, dengue virus, and Zika virus, a protein stabilizer, such as silk fibroin, gelatin, albumin, or a combination thereof, and a sugar or sugar alcohol, such as sucrose, trehalose, sorbitol, mannitol, or a combination thereof. In certain embodiments, the invention provides a substantially dried stabilized vaccine formulation comprising a flavivirus antigen chosen from yellow fever virus, Japanese encephalitis virus, dengue virus, and Zika virus, a protein stabilizer chosen from silk fibroin, gelatin, albumin, or a combination thereof, and a sugar or sugar alcohol chosen from sucrose, trehalose, sorbitol, mannitol, or a combination thereof. In certain embodiments, the invention provides an air-dried stabilized vaccine formulation comprising a flavivirus antigen chosen from yellow fever virus, Japanese encephalitis virus, dengue virus, and Zika virus, a protein stabilizer chosen from silk fibroin and gelatin, and a sugar excipient chosen from sucrose, trehalose, and mannitol. In certain embodiments, the substantially dried stabilized vaccine formulation is lyophilized. In certain embodiments, the substantially dried stabilized vaccine formulation is air-dried. In certain embodiments, the substantially dried stabilized vaccine formulation is air-dried with secondary drying. In certain embodiments, the substantially dried stabilized vaccine formulation retains significant bioactivity when stored at 45° C. for up to two months. In certain embodiments, the substantially dried stabilized vaccine formulation retains significant bioactivity when stored at approximately 25° C. for up to two years.

In certain embodiments, the invention provides an air-dried stabilized vaccine formulation comprising a flavivirus antigen, a protein stabilizer, such as silk fibroin, gelatin, albumin, or a combination thereof, and a sugar or sugar alcohol, such as sucrose, trehalose, sorbitol, mannitol, or a combination thereof. In certain embodiments, the invention provides an air-dried stabilized vaccine formulation comprising a flavivirus antigen, a protein stabilizer chosen from silk fibroin, gelatin, albumin, or a combination thereof, and a sugar or sugar alcohol chosen from sucrose, trehalose, sorbitol, mannitol, or a combination thereof. In certain embodiments, the invention provides an air-dried stabilized vaccine formulation comprising a flavivirus antigen, a protein stabilizer chosen from silk fibroin and gelatin, and a sugar excipient chosen from sucrose, trehalose, and mannitol. In certain embodiments, the air-dried stabilized vaccine formulation is air-dried with secondary drying. In certain embodiments, the air-dried stabilized vaccine formulation retains significant bioactivity when stored at 45° C. for up to one month. In certain embodiments, the air-dried stabilized vaccine formulation retains significant bioactivity when stored at approximately 25° C. for up to two years.

In certain embodiments, the invention provides an air-dried stabilized vaccine formulation comprising a flavivirus antigen chosen from yellow fever virus, Japanese encephalitis virus, dengue virus, and Zika virus, a protein stabilizer, such as silk fibroin, gelatin, albumin, or a combination thereof, and a sugar or sugar alcohol, such as sucrose, trehalose, sorbitol, mannitol, or a combination thereof. In certain embodiments, the invention provides an air-dried stabilized vaccine formulation comprising a flavivirus antigen chosen from yellow fever virus, Japanese encephalitis virus, dengue virus, and Zika virus, a protein stabilizer chosen from silk fibroin, gelatin, albumin, or a combination thereof, and a sugar or sugar alcohol chosen from sucrose, trehalose, sorbitol, mannitol, or a combination thereof. In certain embodiments, the invention provides an air-dried stabilized vaccine formulation comprising a flavivirus antigen chosen from yellow fever virus, Japanese encephalitis virus, dengue virus, and Zika virus, a protein stabilizer chosen from silk fibroin and gelatin, and a sugar excipient chosen from sucrose, trehalose, and mannitol. In certain embodiments, the air-dried stabilized vaccine formulation is air-dried with secondary drying. In certain embodiments, the air-dried stabilized vaccine formulation retains significant bioactivity when stored at 45° C. for up to two months. In certain embodiments, the air-dried stabilized vaccine formulation retains significant bioactivity when stored at approximately 25° C. for up to two years.

In certain embodiments, the invention provides an air-dried stabilized vaccine formulation comprising a yellow fever antigen, a protein stabilizer, such as silk fibroin, gelatin, albumin, or a combination thereof, and a sugar or sugar alcohol, such as sucrose, trehalose, sorbitol, mannitol, or a combination thereof. In certain embodiments, the invention provides an air-dried stabilized vaccine formulation comprising a yellow fever antigen, a protein stabilizer chosen from silk fibroin, gelatin, albumin, or a combination thereof, and a sugar or sugar alcohol chosen from sucrose, trehalose, sorbitol, mannitol, or a combination thereof. In certain embodiments, the invention provides an air-dried stabilized vaccine formulation comprising a yellow fever antigen, a protein stabilizer chosen from silk fibroin and gelatin, and a sugar excipient chosen from sucrose and trehalose. In certain embodiments, the invention provides an air-dried stabilized vaccine formulation comprising a yellow fever antigen, silk fibroin, gelatin, sucrose, and sorbitol. In certain embodiments, the invention provides an air-dried stabilized vaccine formulation comprising a yellow fever antigen, silk fibroin, gelatin, trehalose, and sorbitol. In certain embodiments, the invention provides an air-dried stabilized vaccine formulation comprising a yellow fever antigen, gelatin, sucrose, and sorbitol. In certain embodiments, the air-dried stabilized vaccine formulation is air-dried with secondary drying. In certain embodiments, the air-dried stabilized vaccine formulation retains significant bioactivity when stored at 45° C. for up to one month. In certain embodiments, the air-dried stabilized vaccine formulation retains significant bioactivity when stored at approximately 25° C. for up to two years.

In certain embodiments, the invention provides an air-dried stabilized vaccine formulation comprising a Japanese encephalitis antigen, a protein stabilizer, such as silk fibroin, gelatin, albumin, or a combination thereof, and a sugar or sugar alcohol, such as sucrose, trehalose, sorbitol, mannitol, or a combination thereof. In certain embodiments, the invention provides an air-dried stabilized vaccine formulation comprising a Japanese encephalitis antigen, a protein stabilizer chosen from silk fibroin, gelatin, albumin, or a combination thereof, and a sugar or sugar alcohol chosen from sucrose, trehalose, sorbitol, mannitol, or a combination thereof. In certain embodiments, the invention provides an air-dried stabilized vaccine formulation comprising a Japanese encephalitis antigen, silk fibroin, albumin, and mannitol. In certain embodiments, the air-dried stabilized vaccine formulation is air-dried with secondary drying. In certain embodiments, the air-dried stabilized vaccine formulation retains significant bioactivity when stored at 45° C. for up to one month. In certain embodiments, the air-dried stabilized vaccine formulation retains significant bioactivity when stored at approximately 25° C. for up to two years.

In certain embodiments, the invention provides an air-dried stabilized vaccine formulation comprising a dengue virus antigen, a protein stabilizer, such as silk fibroin, gelatin, albumin, or a combination thereof, and a sugar or sugar alcohol, such as sucrose, trehalose, sorbitol, mannitol, or a combination thereof. In certain embodiments, the invention provides an air-dried stabilized vaccine formulation comprising a dengue virus antigen, a protein stabilizer chosen from silk fibroin, gelatin, albumin, or a combination thereof, and a sugar or sugar alcohol chosen from sucrose, trehalose, sorbitol, mannitol, or a combination thereof. In certain embodiments, the invention provides an air-dried stabilized vaccine formulation comprising a dengue virus antigen, a protein stabilizer chosen from silk fibroin and gelatin, and a sugar excipient chosen from sucrose, trehalose, and mannitol. In certain embodiments, the air-dried stabilized vaccine formulation is air-dried with secondary drying. In certain embodiments, the air-dried stabilized vaccine formulation retains significant bioactivity when stored at 45° C. for up to one month. In certain embodiments, the air-dried stabilized vaccine formulation retains significant bioactivity when stored at approximately 25° C. for up to two years. In certain embodiments, the invention provides an air-dried stabilized vaccine formulation comprising a Zika virus antigen, a protein stabilizer, such as silk fibroin, gelatin, albumin, or a combination thereof, and a sugar or sugar alcohol, such as sucrose, trehalose, sorbitol, mannitol, or a combination thereof. In certain embodiments, the invention provides an air-dried stabilized vaccine formulation comprising a Zika virus antigen, a protein stabilizer chosen from silk fibroin, gelatin, albumin, or a combination thereof, and a sugar or sugar alcohol chosen from sucrose, trehalose, sorbitol, mannitol, or a combination thereof. In certain embodiments, the invention provides an air-dried stabilized vaccine formulation comprising a Zika virus antigen, a protein stabilizer chosen from silk fibroin and gelatin, and a sugar excipient chosen from sucrose, trehalose, and mannitol. In certain embodiments, the air-dried stabilized vaccine formulation is air-dried with secondary drying. In certain embodiments, the air-dried stabilized vaccine formulation retains significant bioactivity when stored at 45° C. for up to one month. In certain embodiments, the air-dried stabilized vaccine formulation retains significant bioactivity when stored at approximately 25° C. for up to two years.

Protein Stabilizers for Flavivirus Vaccines

In certain embodiments, the vaccine preparations of the invention include at least one protein stabilizer which aids in retaining the bioactivity of the vaccine antigens. In some embodiments, the protein stabilizer is selected from the group consisting of silk fibroin, gelatin, and albumin, or a combination thereof.

In certain embodiments, the invention relates to any one of the liquid formulations described herein, wherein the amount of protein chosen from silk fibroin, albumin, gelatin, or a combination thereof, in the formulation is from 0.05 milligrams to 100 milligrams per standard dose. In certain embodiments, the invention relates to any one of the liquid formulations described herein, wherein the amount of protein chosen from silk fibroin, albumin, gelatin, or a combination thereof, in the formulation is from 0.05 milligrams to 75 milligrams per standard dose. In certain embodiments, the invention relates to any one of the liquid formulations described herein, wherein the amount of protein chosen from silk fibroin, albumin, gelatin, or a combination thereof, in the formulation is from 0.05 milligrams to 50 milligrams per standard dose. In certain embodiments, the invention relates to any one of the liquid formulations described herein, wherein the amount of protein chosen from silk fibroin, albumin, gelatin, or a combination thereof, in the formulation is from 0.25 milligrams to 100 milligrams per standard dose. In certain embodiments, the invention relates to any one of the liquid formulations described herein, wherein the amount of protein chosen from silk fibroin, albumin, gelatin, or a combination thereof, in the formulation is from 0.25 milligrams to 75 milligrams per standard dose. In certain embodiments, the invention relates to any one of the liquid formulations described herein, wherein the amount of protein chosen from silk fibroin, albumin, gelatin, or a combination thereof, in the formulation is from 0.25 milligrams to 50 milligrams per standard dose.

In certain embodiments, the invention relates to any one of the liquid formulations described herein, wherein the amount of silk fibroin in the formulation is from 0.5 milligrams to 100 milligrams per standard dose. In certain embodiments, the invention relates to any one of the liquid formulations described herein, wherein the amount of silk fibroin in the formulation is from 2.5 milligrams to 75 milligrams per standard dose. In certain embodiments, the invention relates to any one of the liquid formulations described herein, wherein the amount of silk fibroin in the formulation is from 5 milligrams to 50 milligrams per standard dose. In certain embodiments, the invention relates to any one of the liquid formulations described herein, wherein the amount of silk fibroin in the formulation is from 5 milligrams to 38.75 milligrams per standard dose.

In certain embodiments, the invention relates to any one of the liquid formulations described herein, wherein the amount of albumin in the formulation is from 0.05 milligrams to 50 milligrams per standard dose. In certain embodiments, the invention relates to any one of the liquid formulations described herein, wherein the amount of albumin in the formulation is from 0.25 milligrams to 25 milligrams per standard dose. In certain embodiments, the invention relates to any one of the liquid formulations described herein, wherein the amount of albumin in the formulation is from 0.25 milligrams to 5 milligrams per standard dose. In certain embodiments, the invention relates to any one of the liquid formulations described herein, wherein the amount of albumin in the formulation is from 0.5 milligrams to 5 milligrams per standard dose.

In certain embodiments, the invention relates to any one of the liquid formulations described herein, wherein the amount of gelatin in the formulation is from 7.5 milligrams to 50 milligrams per standard dose. In certain embodiments, the invention relates to any one of the liquid formulations described herein, wherein the amount of gelatin in the formulation is from 7.5 milligrams to 25 milligrams per standard dose. In certain embodiments, the invention relates to any one of the liquid formulations described herein, wherein the amount of gelatin in the formulation is about 12.5 milligrams.

In certain embodiments, the invention relates to any of the liquid formulations described herein, wherein the amount of silk fibroin in the formulation is from 0.1% (w/v) to 20% (w/v). In certain embodiments, the invention relates to any of the liquid formulations described herein, wherein the amount of silk fibroin in the formulation is from 0.5% (w/v) to 15% (w/v). In certain embodiments, the invention relates to any of the liquid formulations described herein, wherein the amount of silk fibroin in the formulation is from 1% (w/v) to 10% (w/v). In certain embodiments, the invention relates to any of the liquid formulations described herein, wherein the amount of silk fibroin in the formulation is from 1% (w/v) to 7.75% (w/v).

In certain embodiments, the invention relates to any of the liquid formulations described herein, wherein the amount of albumin in the formulation is from 0.01% (w/v) to 10% (w/v). In certain embodiments, the invention relates to any of the liquid formulations described herein, wherein the amount of albumin in the formulation is from 0.05% (w/v) to 5% (w/v). In certain embodiments, the invention relates to any of the liquid formulations described herein, wherein the amount of albumin in the formulation is from 0.05% (w/v) to 1% (w/v). In certain embodiments, the invention relates to any of the liquid formulations described herein, wherein the amount of albumin in the formulation is from 0.1% (w/v) to 1% (w/v).

In certain embodiments, the invention relates to any of the liquid formulations described herein, wherein the amount of gelatin in the formulation is over 1.5% (w/v) and up to 10% (w/v). In certain embodiments, the invention relates to any of the liquid formulations described herein, wherein the amount of gelatin in the formulation is over 1.5% (w/v) and up to 5% (w/v). In certain embodiments, the invention relates to any of the liquid formulations described herein, wherein the amount of gelatin in the formulation is about 2.5% (w/v).

In certain embodiments, the invention relates to any one of the substantially dried formulations described herein, wherein the amount of protein chosen from silk fibroin, gelatin, albumin, or a combination thereof, in the formulation is from 0.5 milligrams to 100 milligrams per standard dose. In certain embodiments, the invention relates to any one of the formulations described herein, wherein the amount of protein chosen from silk fibroin, gelatin, albumin, or a combination thereof, in the formulation is from 2.5 milligrams to 50 milligrams per standard dose. In certain embodiments, the invention relates to any one of the formulations described herein, wherein the amount of protein chosen from silk fibroin, gelatin, albumin, or a combination thereof, in the formulation is from 2.5 milligrams to 32.5 milligrams per standard dose. In certain embodiments, the invention relates to any one of the formulations described herein, wherein the amount of protein chosen from silk fibroin, gelatin, albumin, or a combination thereof, in the formulation is from 5 milligrams to 32.5 milligrams per standard dose. In certain embodiments, the invention relates to any one of the formulations described herein, wherein the amount of protein chosen from silk fibroin, gelatin, albumin, or a combination thereof, in the formulation is from 7.5 milligrams to 32.5 milligrams per standard dose.

In certain embodiments, the invention relates to any one of the substantially dried formulations described herein, wherein the amount of protein chosen from silk fibroin, gelatin, albumin, or a combination thereof, in the formulation is from 0.001 milligrams to 2 grams. In certain embodiments, the invention relates to any one of the formulations described herein, wherein the amount of protein chosen from silk fibroin, gelatin, albumin, or a combination thereof, in the formulation is from 0.0025 milligrams to 1 gram. In certain embodiments, the invention relates to any one of the formulations described herein, wherein the amount of protein chosen from silk fibroin, gelatin, albumin, or a combination thereof, in the formulation is from 0.0025 milligrams to 650 milligrams. In certain embodiments, the invention relates to any one of the formulations described herein, wherein the amount of protein chosen from silk fibroin, gelatin, albumin, or a combination thereof, in the formulation is from 0.005 milligrams to 650 milligrams. In certain embodiments, the invention relates to any one of the formulations described herein, wherein the amount of protein chosen from silk fibroin, gelatin, albumin, or a combination thereof, in the formulation is from 0.0075 milligrams to 650 milligrams.

In certain embodiments, the invention relates to any one of the substantially dried formulations described herein, wherein the amount of protein chosen from silk fibroin, gelatin, albumin, or a combination thereof, in the formulation immediately before drying is from 0.1% (w/v) to 20% (w/v). In certain embodiments, the invention relates to any one of the substantially dried formulations described herein, wherein the amount of protein chosen from silk fibroin, gelatin, albumin, or a combination thereof, in the formulation immediately before drying is from 0.5% (w/v) to 10% (w/v). In certain embodiments, the invention relates to any one of the substantially dried formulations described herein, wherein the amount of protein chosen from silk fibroin, gelatin, albumin, or a combination thereof, in the formulation immediately before drying is from 0.5% (w/v) to 6.5% (w/v). In certain embodiments, the invention relates to any one of the substantially dried formulations described herein, wherein the amount of protein chosen from silk fibroin, gelatin, albumin, or a combination thereof, in the formulation immediately before drying is from 1% (w/v) to 6.5% (w/v). In certain embodiments, the invention relates to any one of the substantially dried formulations described herein, wherein the amount of protein chosen from silk fibroin, gelatin, albumin, or a combination thereof, in the formulation immediately before drying is over 1.5% (w/v) and less than 6.5% (w/v).

Hydrolyzed gelatin (Gelita VacciPro®, Sergeant Bluff, Iowa) was prepared at 10% (w/v) by dissolving dry mass in reduced volume of water at 60° C. and adding water to achieve desired concentration. The solution was then sterile-filtered (0.2 μm) prior to formulation.

Bovine serum albumin (Sigma-Aldrich, St. Louis, Mo.; product #A3294) was prepared at 10% (w/v) by dissolving dry mass in reduced volume of water and adding water to achieve desired concentration. The solution was then sterile-filtered (0.2 μm) prior to formulation.

Sugar and Sugar Alcohol Excipients for Flavivirus Vaccines

In certain embodiments, the vaccine preparations of the invention include at least one sugar or sugar alcohol excipient. In some embodiments, the sugar or sugar alcohol is selected from the group consisting of sucrose, trehalose, mannitol, and sorbitol, or a combination thereof. In some embodiments, the sugar or sugar alcohol is selected from the group consisting of sucrose, trehalose, and mannitol.

In certain embodiments, the invention relates to any of the substantially dried formulations described herein, wherein the amount of sugar chosen from sucrose, trehalose, mannitol, or sorbitol, or a combination thereof, in the formulation is over 7.5 milligrams and up to 100 milligrams per standard dose. In certain embodiments, the invention relates to any of the substantially dried formulations described herein, wherein the amount of sugar chosen from sucrose, trehalose, mannitol, or sorbitol, or a combination thereof, in the formulation is over 7.5 milligrams and up to 75 milligrams per standard dose. In certain embodiments, the invention relates to any of the substantially dried formulations described herein, wherein the amount of sugar chosen from sucrose, trehalose, mannitol, or sorbitol, or a combination thereof, in the formulation is over 7.5 milligrams and up to 50 milligrams per standard dose. In certain embodiments, the invention relates to any of the substantially dried formulations described herein, wherein the amount of sugar chosen from sucrose, trehalose, mannitol, or sorbitol, or a combination thereof, in the formulation is over 12.5 milligrams and up to 50 milligrams per standard dose. In certain embodiments, the invention relates to any of the substantially dried formulations described herein, wherein the amount of sugar chosen from sucrose, trehalose, mannitol, or sorbitol, or a combination thereof, in the formulation is over 25 milligrams and up to 50 milligrams per standard dose.

In certain embodiments, the invention relates to any of the substantially dried formulations described herein, wherein the amount of sugar chosen from sucrose, trehalose, mannitol, or sorbitol, or a combination thereof, in the formulation is from 0.0075 milligrams to 2 grams. In certain embodiments, the invention relates to any of the substantially dried formulations described herein, wherein the amount of sugar chosen from sucrose, trehalose, mannitol, or sorbitol, or a combination thereof, in the formulation is from 0.0075 milligrams to 1.5 grams. In certain embodiments, the invention relates to any of the substantially dried formulations described herein, wherein the amount of sugar chosen from sucrose, trehalose, mannitol, or sorbitol, or a combination thereof, in the formulation is from 0.0075 milligrams to 1 gram. In certain embodiments, the invention relates to any of the substantially dried formulations described herein, wherein the amount of sugar chosen from sucrose, trehalose, mannitol, or sorbitol, or a combination thereof, in the formulation is from 0.0125 milligrams to 1 gram. In certain embodiments, the invention relates to any of the substantially dried formulations described herein, wherein the amount of sugar chosen from sucrose, trehalose, mannitol, or sorbitol, or a combination thereof, in the formulation is from 0.025 milligrams to 1 gram.

In certain embodiments, the invention relates to any one of the substantially dried formulations described herein, wherein the amount sugar chosen from sucrose, trehalose, mannitol, or sorbitol, or a combination thereof, in the formulation immediately before drying is over 1.5% (w/v) and up to 20% (w/v). In certain embodiments, the invention relates to any one of the substantially dried formulations described herein, wherein the amount sugar chosen from sucrose, trehalose mannitol, or sorbitol, or a combination thereof, in the formulation immediately before drying is over 1.5% (w/v) and up to 15% (w/v). In certain embodiments, the invention relates to any one of the substantially dried formulations described herein, wherein the amount of sugar chosen from sucrose, trehalose, mannitol, or sorbitol, or a combination thereof, in the formulation immediately before drying is over 1.5% (w/v) and up to 10% (w/v). In certain embodiments, the invention relates to any one of the substantially dried formulations described herein, wherein the amount of sugar chosen from sucrose, trehalose, mannitol, or sorbitol, or a combination thereof, in the formulation immediately before drying is over 2.5% (w/v) and up to 10% (w/v). In certain embodiments, the invention relates to any one of the substantially dried formulations described herein, wherein the amount of sugar chosen from sucrose, trehalose, mannitol, or sorbitol, or a combination thereof, in the formulation immediately before drying is over 5% (w/v) and up to 10% (w/v).

In certain embodiments, the invention relates to any of the substantially dried formulations described herein, wherein the amount of sugar chosen from sucrose, trehalose, and mannitol in the formulation is over 5 milligrams and up to 100 milligrams per standard dose. In certain embodiments, the invention relates to any of the substantially dried formulations described herein, wherein the amount of sugar chosen from sucrose, trehalose, and mannitol in the formulation is over 5 milligrams and up to 75 milligrams per standard dose. In certain embodiments, the invention relates to any of the substantially dried formulations described herein, wherein the amount of sugar chosen from sucrose, trehalose, and mannitol in the formulation is over 5 milligrams and up to 50 milligrams per standard dose.

In certain embodiments, the invention relates to any of the substantially dried formulations described herein, wherein the amount of sugar chosen from sucrose, trehalose, and mannitol in the formulation is from 0.005 milligrams to 2 grams. In certain embodiments, the invention relates to any of the substantially dried formulations described herein, wherein the amount of sugar chosen from sucrose, trehalose, and mannitol in the formulation is from 0.005 milligrams to 1.5 grams. In certain embodiments, the invention relates to any of the substantially dried formulations described herein, wherein the amount of sugar chosen from sucrose, trehalose, and mannitol in the formulation is from 0.005 milligrams to 1 gram.

In certain embodiments, the invention relates to any one of the substantially dried formulations described herein, wherein the amount sugar chosen from sucrose, trehalose, and mannitol in the formulation immediately before drying is over 1% (w/v) and up to 20% (w/v). In certain embodiments, the invention relates to any one of the substantially dried formulations described herein, wherein the amount sugar chosen from sucrose, trehalose, and mannitol in the formulation immediately before drying is over 1% (w/v) and up to 15% (w/v). In certain embodiments, the invention relates to any one of the substantially dried formulations described herein, wherein the amount of sugar chosen from sucrose, trehalose, and mannitol in the formulation immediately before drying is over 1% (w/v) and up to 10% (w/v).

pH of Flavivirus Vaccine Formulation

In certain embodiments, the invention relates to any one of the liquid formulations described herein, wherein the formulation has a pH lower than 6.7. In certain embodiments, the invention relates to any one of the substantially dried formulations described herein, wherein the formulation has a pH before drying lower than 6.7.

In certain embodiments, the invention relates to any one of the liquid formulations described herein, wherein the formulation has a pH lower than 6.7 and higher than 6.2. In certain embodiments, the invention relates to any one of the substantially dried formulations described herein, wherein the formulation has a pH before drying lower than 6.7 and higher than 6.2.

Drying and Water Content of Flavivirus Vaccines

In certain embodiments, the invention relates to any one of the formulations described herein, wherein the formulation is an air-dried formulation. In certain embodiments, the invention relates to any one of the formulations described herein, wherein the formulation has been air-dried at a temperature of from 2° C. to 50° C. In certain embodiments, the invention relates to any one of the formulations described herein, wherein the formulation has been air-dried at a temperature of 2-5° C., 5-10° C., 10-15° C., 15-20° C., 20-25° C., 25-30° C., 30-35° C., 35-40° C., or 40-45° C. In certain embodiments, the invention relates to any one of the formulations described herein, wherein the formulation has been air-dried at a temperature of about 23° C.

In certain embodiments, the invention relates to any one of the formulations described herein, wherein the formulation is an air-dried formulation with secondary drying, meaning that after completion of air-drying, the formulation is subjected to a second prescribed drying cycle. For example, in some specific embodiments, the formulation is subjected to a secondary drying cycle by (1) holding at 10° C. to 20° C. (e.g., about 15° C.) under atmospheric or higher pressure (e.g., 750-900 mT) for 30 minutes or more, then (2) lowering temperature to −10° C. to 0° C. (e.g., −5° C.) and holding under vacuum (e.g., ˜50 mT) for 30 minutes or more, and finally (3) progressively increasing the temperature under vacuum (e.g., holding at 10° C., 20° C., then 30° C. for one hour or more, respectively). Those of skill in the art can adjust drying pressures and temperatures for best results or mere convenience.

In certain embodiments, the invention relates to any one of the formulations described herein, wherein the formulation is in the form of a lyophilized powder. For example, in some specific embodiments, the formulation is lyophilized by (1) freezing at −55° C. to −45° C. (e.g., −50° C.) and holding for 1 hour or more, followed by (2) sublimation (primary drying) at −45° C. to −35° C. for ˜3 hours to several days under vacuum (˜45-50 microbar), and (3) desorption (secondary drying) at 25-30 ° C. for ˜3 hours to several days under vacuum (˜10-50 microbar). Those of skill in the art can adjust drying pressures and temperatures for best results or mere convenience.

In certain embodiments, the invention relates to any one of the substantially dry formulations described herein, wherein the formulation is in the form of a film, for example, an air-dried film.

In certain embodiments, the invention relates to any one of the substantially dry formulations described herein, wherein the formulation comprises 0% to 5% by mass water. These formulations with low water content (i.e., less than 5%) are most typically produced by lyophilization, but can be produced by vacuum-drying or air-drying. In certain embodiments, the invention relates to any one of the substantially dry formulations described herein, wherein the formulation comprises water in an amount less than 5% by mass. In certain embodiments, the invention relates to any one of the substantially dry formulations described herein, wherein the formulation comprises water in an amount less than 4% by mass. In certain embodiments, the invention relates to any one of the substantially dry formulations described herein, wherein the formulation comprises water in an amount less than 3% by mass. In certain embodiments, the invention relates to any one of the substantially dry formulations described herein, wherein the formulation comprises water in an amount less than 2% by mass. In certain embodiments, the invention relates to any one of the substantially dry formulations described herein, wherein the formulation comprises water in an amount less than 1% by mass. In certain embodiments, the invention relates to any one of the substantially dry formulations described herein, wherein the formulation comprises water in an amount less than 0.5% by mass.

In certain embodiments, the invention relates to any one of the substantially dry formulations described herein, wherein the formulation comprises water in an amount between 5% and 20%. These substantially dry formulations with higher water content (i.e., 5%-20%) are preferably produced by air-drying, but can be produced by vacuum-drying or partial lyophilization. Thus, in certain embodiments, the formulations comprise greater than 5%, greater than 6%, greater than 7%, greater than 8%, greater than 9%, greater than 10%, greater than 11%, greater than 12%, greater than 13%, greater than 14%, greater than 15%, greater than 16%, greater than 17%, greater than 18%, or greater than 19%, but in each case less than 20% by mass.

Stability and Bioactivity of Flavivirus Vaccines

In certain embodiments, the invention relates to any one of the liquid stabilized vaccine formulations described herein, wherein the formulation retains at least 60% of its original bioactivity after storage at 4° C. for 3 weeks. In certain embodiments, the invention relates to any one of the liquid stabilized vaccine formulations described herein, wherein the formulation retains at least 65% of its original bioactivity after storage at 4° C. for 3 weeks.

In certain embodiments, the invention relates to any one of the liquid stabilized vaccine formulations described herein, wherein the formulation retains at least 30% of its original bioactivity after storage at 4° C. for 4 weeks. In certain embodiments, the invention relates to any one of the liquid stabilized vaccine formulations described herein, wherein the formulation retains at least 40% of its original bioactivity after storage at 4° C. for 4 weeks. In certain embodiments, the invention relates to any one of the liquid stabilized vaccine formulations described herein, wherein the formulation retains at least 50% of its original bioactivity after storage at 4° C. for 4 weeks. In certain embodiments, the invention relates to any one of the liquid stabilized vaccine formulations described herein, wherein the formulation retains at least 60% of its original bioactivity after storage at 4° C. for 4 weeks. In certain embodiments, the invention relates to any one of the liquid stabilized vaccine formulations described herein, wherein the formulation retains at least 65% of its original bioactivity after storage at 4° C. for 4 weeks.

In certain embodiments, the invention relates to any one of the liquid stabilized vaccine formulations described herein, wherein the formulation retains at least 30% of its original bioactivity after storage at 4° C. for 5 weeks. In certain embodiments, the invention relates to any one of the liquid stabilized vaccine formulations described herein, wherein the formulation retains at least 40% of its original bioactivity after storage at 4° C. for 5 weeks. In certain embodiments, the invention relates to any one of the liquid stabilized vaccine formulations described herein, wherein the formulation retains at least 50% of its original bioactivity after storage at 4° C. for 5 weeks. In certain embodiments, the invention relates to any one of the liquid stabilized vaccine formulations described herein, wherein the formulation retains at least 55% of its original bioactivity after storage at 4° C. for 5 weeks.

In certain embodiments, the invention relates to any one of the liquid stabilized vaccine formulations described herein, wherein the formulation retains at least 30% of its original bioactivity after storage at 4° C. for one year. In certain embodiments, the invention relates to any one of the liquid stabilized vaccine formulations described herein, wherein the formulation retains at least 40% of its original bioactivity after storage at 4° C. for one year. In certain embodiments, the invention relates to any one of the liquid stabilized vaccine formulations described herein, wherein the formulation retains at least 50% of its original bioactivity after storage at 4° C. for one year.

In certain embodiments, the invention relates to any one of the liquid stabilized vaccine formulations described herein, wherein the formulation retains at least 30% of its original bioactivity after storage at 4° C. for two years. In certain embodiments, the invention relates to any one of the liquid stabilized vaccine formulations described herein, wherein the formulation retains at least 40% of its original bioactivity after storage at 4° C. for two years. In certain embodiments, the invention relates to any one of the liquid stabilized vaccine formulations described herein, wherein the formulation retains at least 50% of its original bioactivity after storage at 4° C. for two years.

In certain embodiments, the invention relates to any one of the liquid stabilized vaccine formulations described herein, wherein the formulation retains at least 80% of its original bioactivity after storage at 25° C. for 24 hours. In certain embodiments, the invention relates to any one of the liquid stabilized vaccine formulations described herein, wherein the formulation retains at least 90% of its original bioactivity after storage at 25° C. for 24 hours.

In certain embodiments, the invention relates to any one of the liquid stabilized vaccine formulations described herein, wherein the formulation retains at least 30% of its original bioactivity after storage at 25° C. for 48 hours. In certain embodiments, the invention relates to any one of the liquid stabilized vaccine formulations described herein, wherein the formulation retains at least 40% of its original bioactivity after storage at 25° C. for 48 hours. In certain embodiments, the invention relates to any one of the liquid stabilized vaccine formulations described herein, wherein the formulation retains at least 50% of its original bioactivity after storage at 25° C. for 48 hours.

In certain embodiments, the invention relates to any one of the liquid stabilized vaccine formulations described herein, wherein the formulation retains at least 60% of its original bioactivity after storage at 25° C. for 4 hours. In certain embodiments, the invention relates to any one of the liquid stabilized vaccine formulations described herein, wherein the formulation retains at least 70% of its original bioactivity after storage at 25° C. for 4 hours. In certain embodiments, the invention relates to any one of the liquid stabilized vaccine formulations described herein, wherein the formulation retains at least 80% of its original bioactivity after storage at 25° C. for 4 hours. In certain embodiments, the invention relates to any one of the liquid stabilized vaccine formulations described herein, wherein the formulation retains at least 90% of its original bioactivity after storage at 25° C. for 4 hours.

In certain embodiments, the invention relates to any one of the liquid stabilized vaccine formulations described herein, wherein the formulation retains at least 60% of its original bioactivity after storage at 25° C. for 8 hours. In certain embodiments, the invention relates to any one of the liquid stabilized vaccine formulations described herein, wherein the formulation retains at least 70% of its original bioactivity after storage at 25° C. for 8 hours. In certain embodiments, the invention relates to any one of the liquid stabilized vaccine formulations described herein, wherein the formulation retains at least 80% of its original bioactivity after storage at 25° C. for 8 hours. In certain embodiments, the invention relates to any one of the liquid stabilized vaccine formulations described herein, wherein the formulation retains at least 90% of its original bioactivity after storage at 25° C. for 8 hours.

In certain embodiments, the invention relates to any one of the liquid stabilized vaccine formulations described herein, wherein the formulation retains at least 60% of its original bioactivity after storage at 25° C. for 12 hours. In certain embodiments, the invention relates to any one of the liquid stabilized vaccine formulations described herein, wherein the formulation retains at least 70% of its original bioactivity after storage at 25° C. for 12 hours. In certain embodiments, the invention relates to any one of the liquid stabilized vaccine formulations described herein, wherein the formulation retains at least 80% of its original bioactivity after storage at 25° C. for 12 hours. In certain embodiments, the invention relates to any one of the liquid stabilized vaccine formulations described herein, wherein the formulation retains at least 90% of its original bioactivity after storage at 25° C. for 12 hours.

In certain embodiments, the invention relates to any one of the liquid stabilized vaccine formulations described herein, wherein the formulation retains at least 70% of its original bioactivity after storage at 37° C. for 4 hours. In certain embodiments, the invention relates to any one of the liquid stabilized vaccine formulations described herein, wherein the formulation retains at least 80% of its original bioactivity after storage at 37° C. for 4 hours.

In certain embodiments, the invention relates to any one of the liquid stabilized vaccine formulations described herein, wherein the formulation retains at least 40% of its original bioactivity after storage at 37° C. for 8 hours. In certain embodiments, the invention relates to any one of the liquid stabilized vaccine formulations described herein, wherein the formulation retains at least 50% of its original bioactivity after storage at 37° C. for 8 hours. In certain embodiments, the invention relates to any one of the liquid stabilized vaccine formulations described herein, wherein the formulation retains at least 60% of its original bioactivity after storage at 37° C. for 8 hours. In certain embodiments, the invention relates to any one of the liquid stabilized vaccine formulations described herein, wherein the formulation retains at least 70% of its original bioactivity after storage at 37° C. for 8 hours.

In certain embodiments, the invention relates to any one of the liquid stabilized vaccine formulations described herein, wherein the formulation retains at least 30% of its original bioactivity after storage at 37° C. for 12 hours. In certain embodiments, the invention relates to any one of the liquid stabilized vaccine formulations described herein, wherein the formulation retains at least 40% of its original bioactivity after storage at 37° C. for 12 hours. In certain embodiments, the invention relates to any one of the liquid stabilized vaccine formulations described herein, wherein the formulation retains at least 45% of its original bioactivity after storage at 37° C. for 12 hours.

In certain embodiments, the invention relates to any one of the liquid stabilized vaccine formulations described herein, wherein the formulation retains at least 30% of its original bioactivity after storage at 37° C. for 13 hours. In certain embodiments, the invention relates to any one of the liquid stabilized vaccine formulations described herein, wherein the formulation retains at least 40% of its original bioactivity after storage at 37° C. for 13 hours. In certain embodiments, the invention relates to any one of the liquid stabilized vaccine formulations described herein, wherein the formulation retains at least 45% of its original bioactivity after storage at 37° C. for 13 hours.

In certain embodiments, the invention relates to any one of the substantially dried vaccine formulations described herein, wherein the formulation retains at least 70% of its original bioactivity after storage at 25° C. for 2 weeks. In certain embodiments, the invention relates to any one of the substantially dried vaccine formulations described herein, wherein the formulation retains at least 80% of its original bioactivity after storage at 25° C. for 2 weeks. In certain embodiments, the invention relates to any one of the substantially dried vaccine formulations described herein, wherein the formulation retains at least 90% of its original bioactivity after storage at 25° C. for 2 weeks.

In certain embodiments, the invention relates to any one of the substantially dried vaccine formulations described herein, wherein the formulation retains at least 60% of its original bioactivity after storage at 25° C. for 4 weeks. In certain embodiments, the invention relates to any one of the substantially dried vaccine formulations described herein, wherein the formulation retains at least 70% of its original bioactivity after storage at 25° C. for 4 weeks. In certain embodiments, the invention relates to any one of the substantially dried vaccine formulations described herein, wherein the formulation retains at least 80% of its original bioactivity after storage at 25° C. for 4 weeks. In certain embodiments, the invention relates to any one of the substantially dried vaccine formulations described herein, wherein the formulation retains at least 90% of its original bioactivity after storage at 25° C. for 4 weeks.

In certain embodiments, the invention relates to any one of the substantially dried vaccine formulations described herein, wherein the formulation retains at least 40% of its original bioactivity after storage at 25° C. for 8 weeks. In certain embodiments, the invention relates to any one of the substantially dried vaccine formulations described herein, wherein the formulation retains at least 50% of its original bioactivity after storage at 25° C. for 8 weeks. In certain embodiments, the invention relates to any one of the substantially dried vaccine formulations described herein, wherein the formulation retains at least 60% of its original bioactivity after storage at 25° C. for 8 weeks. In certain embodiments, the invention relates to any one of the substantially dried vaccine formulations described herein, wherein the formulation retains at least 70% of its original bioactivity after storage at 25° C. for 8 weeks. In certain embodiments, the invention relates to any one of the substantially dried vaccine formulations described herein, wherein the formulation retains at least 80% of its original bioactivity after storage at 25° C. for 8 weeks.

In certain embodiments, the invention relates to any one of the substantially dried vaccine formulations described herein, wherein the formulation retains at least 40% of its original bioactivity after storage at 25° C. for 12 weeks. In certain embodiments, the invention relates to any one of the substantially dried vaccine formulations described herein, wherein the formulation retains at least 50% of its original bioactivity after storage at 25° C. for 12 weeks. In certain embodiments, the invention relates to any one of the substantially dried vaccine formulations described herein, wherein the formulation retains at least 60% of its original bioactivity after storage at 25° C. for 12 weeks. In certain embodiments, the invention relates to any one of the substantially dried vaccine formulations described herein, wherein the formulation retains at least 70% of its original bioactivity after storage at 25° C. for 12 weeks. In certain embodiments, the invention relates to any one of the substantially dried vaccine formulations described herein, wherein the formulation retains at least 80% of its original bioactivity after storage at 25° C. for 12 weeks.

In certain embodiments, the invention relates to any one of the substantially dried vaccine formulations described herein, wherein the formulation retains at least 30% of its original bioactivity after storage at 25° C. for 1 year. In certain embodiments, the invention relates to any one of the substantially dried vaccine formulations described herein, wherein the formulation retains at least 40% of its original bioactivity after storage at 25° C. for 1 year. In certain embodiments, the invention relates to any one of the substantially dried vaccine formulations described herein, wherein the formulation retains at least 50% of its original bioactivity after storage at 25° C. for 1 year. In certain embodiments, the invention relates to any one of the substantially dried vaccine formulations described herein, wherein the formulation retains at least 60% of its original bioactivity after storage at 25° C. for 1 year.

In certain embodiments, the invention relates to any one of the substantially dried vaccine formulations described herein, wherein the formulation retains at least 30% of its original bioactivity after storage at 25° C. for 2 years. In certain embodiments, the invention relates to any one of the substantially dried vaccine formulations described herein, wherein the formulation retains at least 40% of its original bioactivity after storage at 25° C. for 2 years. In certain embodiments, the invention relates to any one of the substantially dried vaccine formulations described herein, wherein the formulation retains at least 50% of its original bioactivity after storage at 25° C. for 2 years. In certain embodiments, the invention relates to any one of the substantially dried vaccine formulations described herein, wherein the formulation retains at least 60% of its original bioactivity after storage at 25° C. for 2 years.

In certain embodiments, the invention relates to any one of the substantially dried vaccine formulations described herein, wherein the formulation retains at least 70% of its original bioactivity after storage at 37° C. for 2 weeks. In certain embodiments, the invention relates to any one of the substantially dried vaccine formulations described herein, wherein the formulation retains at least 80% of its original bioactivity after storage at 37° C. for 2 weeks. In certain embodiments, the invention relates to any one of the substantially dried vaccine formulations described herein, wherein the formulation retains at least 90% of its original bioactivity after storage at 37° C. for 2 weeks.

In certain embodiments, the invention relates to any one of the substantially dried vaccine formulations described herein, wherein the formulation retains at least 60% of its original bioactivity after storage at 37° C. for 4 weeks. In certain embodiments, the invention relates to any one of the substantially dried vaccine formulations described herein, wherein the formulation maintains at least 70% of its original bioactivity after storage at 37° C. for 4 weeks. In certain embodiments, the invention relates to any one of the substantially dried vaccine formulations described herein, wherein the formulation maintains at least 80% of its original bioactivity after storage at 37° C. for 4 weeks. In certain embodiments, the invention relates to any one of the substantially dried vaccine formulations described herein, wherein the formulation maintains at least 90% of its original bioactivity after storage at 37° C. for 4 weeks.

In certain embodiments, the invention relates to any one of the substantially dried vaccine formulations described herein, wherein the formulation retains at least 40% of its original bioactivity after storage at 37° C. for 8 weeks. In certain embodiments, the invention relates to any one of the substantially dried vaccine formulations described herein, wherein the formulation retains at least 50% of its original bioactivity after storage at 37° C. for 8 weeks. In certain embodiments, the invention relates to any one of the substantially dried vaccine formulations described herein, wherein the formulation maintains at least 60% of its original bioactivity after storage at 37° C. for 8 weeks. In certain embodiments, the invention relates to any one of the substantially dried vaccine formulations described herein, wherein the formulation maintains at least 70% of its original bioactivity after storage at 37° C. for 8 weeks. In certain embodiments, the invention relates to any one of the substantially dried vaccine formulations described herein, wherein the formulation maintains at least 80% of its original bioactivity after storage at 37° C. for 8 weeks.

In certain embodiments, the invention relates to any one of the substantially dried vaccine formulations described herein, wherein the formulation retains at least 40% of its original bioactivity after storage at 37° C. for 12 weeks. In certain embodiments, the invention relates to any one of the substantially dried vaccine formulations described herein, wherein the formulation retains at least 50% of its original bioactivity after storage at 37° C. for 12 weeks. In certain embodiments, the invention relates to any one of the substantially dried vaccine formulations described herein, wherein the formulation retains at least 60% of its original bioactivity after storage at 37° C. for 12 weeks. In certain embodiments, the invention relates to any one of the substantially dried vaccine formulations described herein, wherein the formulation retains at least 70% of its original bioactivity after storage at 37° C. for 12 weeks. In certain embodiments, the invention relates to any one of the substantially dried vaccine formulations described herein, wherein the formulation retains at least 80% of its original bioactivity after storage at 37° C. for 12 weeks.

In certain embodiments, the invention relates to any one of the substantially dried vaccine formulations described herein, wherein the formulation maintains at least 30% of its original bioactivity after storage at 37° C. for 6 months. In certain embodiments, the invention relates to any one of the substantially dried vaccine formulations described herein, wherein the formulation retains at least 40% of its original bioactivity after storage at 37° C. for 6 months. In certain embodiments, the invention relates to any one of the substantially dried vaccine formulations described herein, wherein the formulation retains at least 50% of its original bioactivity after storage at 37° C. for 6 months. In certain embodiments, the invention relates to any one of the substantially dried vaccine formulations described herein, wherein the formulation retains at least 60% of its original bioactivity after storage at 37° C. for 6 months.

In certain embodiments, the invention relates to any one of the substantially dried vaccine formulations described herein, wherein the formulation retains at least 70% of its original bioactivity after storage at 45° C. for 2 weeks. In certain embodiments, the invention relates to any one of the substantially dried vaccine formulations described herein, wherein the formulation retains at least 80% of its original bioactivity after storage at 45° C. for 2 weeks. In certain embodiments, the invention relates to any one of the substantially dried vaccine formulations described herein, wherein the formulation retains at least 90% of its original bioactivity after storage at 45° C. for 2 weeks.

In certain embodiments, the invention relates to any one of the substantially dried vaccine formulations described herein, wherein the formulation retains at least 60% of its original bioactivity after storage at 45° C. for 4 weeks. In certain embodiments, the invention relates to any one of the substantially dried vaccine formulations described herein, wherein the formulation retains at least 70% of its original bioactivity after storage at 45° C. for 4 weeks. In certain embodiments, the invention relates to any one of the substantially dried vaccine formulations described herein, wherein the formulation maintains at least 80% of its original bioactivity after storage at 45° C. for 4 weeks. In certain embodiments, the invention relates to any one of the substantially dried vaccine formulations described herein, wherein the formulation maintains at least 90% of its original bioactivity after storage at 45° C. for 4 weeks.

In certain embodiments, the invention relates to any one of the substantially dried vaccine formulations described herein, wherein the formulation retains at least about 30% of its original bioactivity after storage at about 45° C. for about 8 weeks. In certain embodiments, the invention relates to any one of the substantially dried vaccine formulations described herein, wherein the formulation retains at least about 40% of its original bioactivity after storage at about 45° C. for about 8 weeks. In certain embodiments, the invention relates to any one of the substantially dried vaccine formulations described herein, wherein the formulation retains at least about 50% of its original bioactivity after storage at about 45° C. for about 8 weeks. In certain embodiments, the invention relates to any one of the substantially dried vaccine formulations described herein, wherein the formulation retains at least 55% of its original bioactivity after storage at 45° C. for 8 weeks.

In certain embodiments, the invention relates to any one of the substantially dried vaccine formulations described herein, wherein the formulation retains at least 40% of its original bioactivity after storage at 45° C. for 12 weeks. In certain embodiments, the invention relates to any one of the substantially dried vaccine formulations described herein, wherein the formulation retains at least 50% of its original bioactivity after storage at 45° C. for 12 weeks. In certain embodiments, the invention relates to any one of the substantially dried vaccine formulations described herein, wherein the formulation retains at least 60% of its original bioactivity after storage at 45° C. for 12 weeks. In certain embodiments, the invention relates to any one of the substantially dried vaccine formulations described herein, wherein the formulation retains at least 70% of its original bioactivity after storage at 45° C. for 12 weeks. In certain embodiments, the invention relates to any one of the substantially dried vaccine formulations described herein, wherein the formulation retains at least 80% of its original bioactivity after storage at 45° C. for 12 weeks.

In certain embodiments, the invention relates to any one of the substantially dried vaccine formulations described herein, wherein the formulation retains at least 30% of its original bioactivity after storage at 45° C. for 6 months. In certain embodiments, the invention relates to any one of the substantially dried vaccine formulations described herein, wherein the formulation retains at least 40% of its original bioactivity after storage at 45° C. for 6 months. In certain embodiments, the invention relates to any one of the substantially dried vaccine formulations described herein, wherein the formulation retains at least 50% of its original bioactivity after storage at 45° C. for 6 months. In certain embodiments, the invention relates to any one of the substantially dried vaccine formulations described herein, wherein the formulation retains at least 60% of its original bioactivity after storage at 45° C. for 6 months.

Reconstitution and/or Administration of Flavivirus Vaccines

In some embodiments, the formulations described herein can be reconstituted in a pharmaceutically acceptable carrier for oral or parenteral administration (e.g., subcutaneous or intramuscular injection). As used herein, the term “pharmaceutically acceptable carrier” refers to any and all solvents, diluents, excipients, dispersion media and the like, which can be used to reconstitute a liquid dosage form.

When administering parenterally, a formulation described herein can be generally presented or reconstituted in a unit dosage injectable form (solution, suspension, emulsion). The formulations suitable for injection include sterile aqueous solutions or dispersions.

The formulations can also contain auxiliary substances such as wetting or emulsifying agents, pH buffering agents, gelling or viscosity enhancing additives, preservatives, colors, and the like, depending upon the route of administration and the preparation desired. Standard texts (e.g., “Remington's Pharmaceutical Science”, 17th edition, 1985, incorporated herein by reference) may be consulted to prepare suitable preparations, without undue experimentation. With respect to formulations described herein, however, any vehicle, diluent, additive or other component used should be biocompatible with the antigens described herein. This will present no problem to those skilled in chemical and pharmaceutical principles, or problems can be readily avoided by reference to standard texts or by simple experiments (not involving undue experimentation).

Exemplary Methods for Preparing Formulations of Flavivirus Vaccines

In some embodiments, the invention relates to a method of preparing any one of the liquid stabilized formulations described herein, comprising the step of:

mixing, in solution, the components of the formulation.

In some embodiments, the invention relates to a method of preparing any one of the substantially dried formulations described herein, comprising the steps of:

mixing, in solution, the components of the formulation; and

lyophilizing the mixture, thereby forming a lyophilized powder or cake.

In some embodiments, the invention relates to any one of the methods described herein, wherein the vaccine mixture is substantially dried, for example, air-dried. In some embodiments, the invention relates to any one of the methods described herein, wherein the vaccine mixture is air-dried to form a substantially dried vaccine mixture in the form of a film.

In some embodiments, the invention relates to any one of the methods described herein, wherein the vaccine mixture is lyophilized. In some embodiments, the invention relates to any one of the methods described herein, wherein the vaccine mixture is lyophilized to form a substantially dried vaccine mixture in the form of a powder.

In some embodiments, the invention relates to a method of preparing any one of the substantially dried formulations described herein, comprising the steps of:

mixing, in solution, the components of the formulation;

air-drying the mixture, thereby forming an air-dried film; and

optionally, subjecting the air-dried film to secondary drying according to a prescribed drying cycle.

In some embodiments, the invention relates to any one of the methods described herein, further comprising the step of:

mixing the substantially dried vaccine mixture with a diluent.

In some embodiments, the invention relates to any one of the methods described herein, further comprising the step of:

preparing the silk fibroin solution from a sample comprising silk fibers from a silkworm Bombyx mori.

The aqueous silk fibroin solution can be prepared using techniques known in the art. Suitable processes for preparing silk fibroin solutions are disclosed, for example, in U.S. Pat. No. 7,635,755; WO 2005/012606; and WO 2008/127401.

In accordance with the conventional practice, the formulations described herein are desirably processed under aseptic conditions using components which preliminarily have been rendered bacterially sterile. Sterility on storage can be maintained by incorporation of an antigen-compatible germicidal substance.

Exemplary Methods of Using Formulations of Flavivirus Vaccines

In certain embodiments, the invention relates to a method of treating or preventing an infection caused by a flavivirus, comprising the step of:

administering to a subject in need thereof a therapeutically or prophylactically effective amount or dose of any one of the formulations described herein, thereby eliciting an immune response in the subject and treating or preventing the infection.

In certain embodiments, the invention relates to any one of the methods described herein, wherein the subject is a mammal.

In certain embodiments, the invention relates to any one of the methods described herein, wherein the subject is a mammal susceptible to or suffering from an infection caused by an flavivirus.

In certain embodiments, the invention relates to any one of the methods described herein, wherein the subject is a human.

In certain embodiments, the invention relates to any one of the methods described herein, wherein the subject is a human over the age of nine months.

In certain embodiments, the invention relates to any one of the methods described herein, wherein the subject is a human between nine months and 17 years of age.

In certain embodiments, the invention relates to any one of the methods described herein, wherein the subject is a human over 18 years of age.

In certain embodiments, the invention relates to any one of the methods described herein, wherein the subject is a human between nine and 45 years of age.

In some embodiments, the invention relates to any one of the methods described herein, wherein the formulation is administered to the subject in one dose. In some embodiments, the invention relates to any one of the methods described herein, wherein the formulation is administered to the subject in two, three, or four spaced doses.

In some embodiments, the invention relates to any one of the methods described herein, wherein the formulation is administered to the subject in two spaced doses. For example, the first dose is administered when the subject is from about 9 months to about 17 years of age, and the second dose is administered between one and two years after the first dose.

In some embodiments, the invention relates to any one of the methods described herein, wherein the formulation is administered to the subject in three spaced doses. For example, after administration of the first dose, the second dose is administered three to nine months (e.g., six months) after the first dose, and the third dose is administered three to nine months (e.g., six months) after the second dose.

In certain embodiments, the invention relates to any one of the methods described herein, wherein the formulation is in the form of a film.

In certain embodiments, the invention relates to any one of the methods described herein, wherein the formulation is in the form of a powder.

In some embodiments, the invention relates to any one of the methods described herein, wherein the formulation is administered to the subject orally.

In certain embodiments, the invention relates to any one of the methods described herein, wherein the formulation is in the form of a film, further comprising the step of: mixing the formulation with a diluent prior to administering to the subject.

In certain embodiments, the invention relates to any one of the methods described herein, wherein the formulation is in the form of a powder, further comprising the step of: mixing the formulation with a diluent prior to administering to the subject.

In some embodiments, the invention relates to any one of the methods described herein, wherein the formulation is administered to the subject by injection, such as subcutaneous, dermal (e.g., transdermal, intradermal or interdermal), or intramuscular injection.

### Exemplary Rotavirus Vaccine Formulations

Overview

Vaccination with rotavirus vaccine has controlled the disease in much of the developed world. Studies evaluating the impact of the introduction of rotavirus vaccine have shown that the vaccine has significantly reduced the burden caused by rotaviral gastroenteritis on healthcare resources. For example, in the United States, studies have shown that rotavirus vaccination reduced rotavirus-associated hospitalizations by 60 to 93%. Studies focused on other regions of the world have shown declines in rotavirus-associated hospitalizations of up to 98% in Europe and up to 83% in Latin America (Dennehy (2012), Curr Opin Pediatr 24:76-84). However, hundreds of thousands of children continue to die each year due to rotavirus, with 85% of these deaths occurring in developing countries in Asia and Africa. In 2009, the World Health Organization recommended that all national immunization programs worldwide include rotavirus vaccination. Estimates indicate that increasing access to rotavirus vaccine in developing countries can prevent more than 2.4 million child deaths by the year 2030 (Tate et al. (2012), Lancet Infect. Dis. 12:136-41). Removing rotavirus vaccine from the constraints of the cold chain would make a significant contribution to the global effort to reduce the incidence of rotavirus infection and acute diarrhea by reducing costs and simplifying logistics related to cold storage and vaccine spoilage.

Currently, two oral rotavirus vaccines are marketed internationally: Rotarix® (GSK Biologicals) is a live monovalent vaccine developed from a G1P[8] rotavirus strain, and RotaTeq® (Merck & Co.) is a pentavalent reassortant vaccine developed from various human and bovine rotavirus strains. Other vaccines not marketed internationally but licensed domestically include: LLV (Lanzhou Institute of Biological Products), an attenuated lamb rotavirus vaccine licensed in China; ROTAVAC® (Bharat Biotech), a live vaccine developed from a human neonatal rotavirus strain that is licensed in India; and Rotavin-M1® (Polyvan), a live monovalent vaccine licensed in Vietnam. Other vaccine candidates currently under clinical development include: BRV-TV (tetravalent) and BRV-PV (pentavalent) (Instituto Butantan; Shantha Biotechnics; Serum Institute of India; Wuhan Institute of Biological Products), two bovine-human reassortant vaccine candidates; RV3-BB (Murdoch Children's Research Institute; Biofarma), a vaccine candidate developed from human neonatal rotavirus strains; and an inactivated rotavirus vaccine candidate developed by the US Centers for Disease Control and Prevention with Sanofi Pasteur. (See, e.g., Vesikari, “Rotavirus Vaccines and Vaccination,” in Viral Gastroenteritis, Svensson et al., Eds., 2016, Elsevier, London.)

Thus, the need exists for improved rotavirus vaccines as described herein.

In certain embodiments, the invention relates to a substantially dried (e.g., lyophilized, vacuum-dried, or air-dried) vaccine formulation comprising, consisting essentially of, or consisting of an antigen, a protein stabilizer, a sugar or a sugar alcohol excipient, a divalent cation, and a buffer salt. In some embodiments, the protein stabilizer is selected from silk fibroin, gelatin, and albumin. In some embodiments, the sugar or the sugar alcohol excipient is selected from sucrose, trehalose, sorbitol, and glycerol, or combinations thereof. In some embodiments, the divalent cation is selected from Ca²⁺, Mg²⁺, Mn²⁺, and Cu²⁺. In some embodiments, the buffer salt is selected from HEPES and citrate phosphate (CP).

In certain embodiments, the invention relates to a substantially dried vaccine formulation comprising, consisting essentially of, or consisting of a C immunogen, a protein, a sugar or a sugar alcohol, a divalent cation salt, and a buffer salt. In some embodiments, the protein is selected from silk fibroin, gelatin and albumin. In some embodiments, the sugar or the sugar alcohol is selected from sucrose, trehalose, sorbitol, and glycerol, or combinations thereof. In some embodiments, the divalent cation salt is magnesium chloride. In some embodiments, the buffer salt is HEPES or CP.

In certain embodiments, the invention relates to a substantially dried vaccine formulation comprising, consisting essentially of, or consisting of a rotavirus immunogen, a protein, a sugar or a sugar alcohol, a divalent cation salt, and a buffer salt, wherein the protein is selected from silk fibroin, gelatin, and albumin; the sugar or the sugar alcohol is selected from sucrose, trehalose, sorbitol, and glycerol, or combinations thereof; and the buffer salt is HEPES or CP.

In certain embodiments, the invention relates to a substantially dried vaccine formulation comprising, consisting essentially of, or consisting of a live attenuated rotavirus, a protein, a sugar or a sugar alcohol, a divalent cation salt, and a buffer salt, wherein the protein is selected from silk fibroin, gelatin, and albumin; the sugar or the sugar alcohol is selected from sucrose, trehalose, sorbitol, and glycerol, or combinations thereof; and the buffer salt is HEPES or CP.

In certain embodiments, the invention relates to a substantially dried vaccine formulation comprising, consisting essentially of, or consisting of a live reassortant rotavirus, a protein, a sugar or a sugar alcohol, a divalent cation salt, and a buffer salt, wherein the protein is selected from silk fibroin, gelatin, and albumin; the sugar or the sugar alcohol is selected from sucrose, trehalose, sorbitol, and glycerol, or combinations thereof; and the buffer salt is HEPES or CP.

In certain embodiments, the invention relates to a substantially dried vaccine formulation comprising, consisting essentially of, or consisting of a rotavirus immunogen, a protein, a sugar or a sugar alcohol, a divalent cation salt, and a buffer salt, wherein the protein is silk fibroin; the sugar or the sugar alcohol is sucrose; the divalent cation salt is calcium chloride; and the buffer salt is HEPES or CP.

In certain embodiments, the invention relates to a substantially dried vaccine formulation comprising, consisting essentially of, or consisting of a live attenuated rotavirus, a protein, a sugar or a sugar alcohol, a divalent cation salt, and a buffer salt, wherein the protein is silk fibroin; the sugar or the sugar alcohol is sucrose; the divalent cation salt is calcium chloride; and the buffer salt is HEPES or CP.

In certain embodiments, the invention relates to a substantially dried vaccine formulation comprising, consisting essentially of, or consisting of a live reassortant rotavirus, a protein, a sugar or a sugar alcohol, a divalent cation salt, and a buffer salt, wherein the protein is silk fibroin; the sugar or the sugar alcohol is sucrose; the divalent cation salt is calcium chloride; and the buffer salt is HEPES or CP.

Rotavirus Immunogens

In certain embodiments, the invention relates to any one of the formulations described herein, wherein the rotavirus immunogen is one or more of the several strains of rotavirus.

In certain embodiments, the rotavirus is live reassortant rotavirus. In certain embodiments, the invention relates to any one of the formulations described herein, wherein the rotavirus is one or more of the several strains of live reassortant rotavirus, including a G1 human reassortant strain, a G2 human reassortant strain, a G3 human reassortant strain, a G4 human reassortant strain, or a P1A[8] human reassortant strain.

Reassortant rotavirus is produced from parent rotavirus strains isolated from hosts of different species, such as human and bovine hosts. Generally, three spaced doses are administered orally to generate adequate levels of seroconversion. Reassortant rotavirus is indicated for the prevention of rotaviral gastroenteritis caused by the human serotypes contained in the vaccine.

In certain embodiments, the invention relates to any one of the formulations described herein, wherein the formulation comprises RotaTeq® (Rotavirus Vaccine, Live, Oral, Pentavalent, produced by Merck & Co.) or an equivalent thereof. RotaTeq® is a sterile suspension of five human-bovine reassortant rotaviruses: four that express the VP7 capsid protein from the human rotavirus parent strain (serotypes G1, G2, G3, or G4, respectively) and the VP4 attachment protein from the bovine rotavirus parent strain (type P7[5] in all cases); and one that expresses the VP4 protein from the human rotavirus parent strain (type P1A[8]) and the VP7 protein from the bovine rotavirus parent strain (serotype G6).

Each dose (2 mL) of RotaTeq® live reassortant rotavirus vaccine is formulated to contain at least 2.2×10⁶ IU of a G1 reassortant strain, 2.8×10⁶ IU of a G2 reassortant strain, 2.2×10⁶ IU of a G3 reassortant strain, 2.0×10⁶ IU of a G4 reassortant strain, and 2.3×10⁶ IU of a P1A[8] reassortant strain. The reassortant rotaviruses are propagated in Vero cells using standard cell culture techniques in the absence of antifungal agents and then suspended in a buffered stabilizer solution. Each vaccine dose contains sucrose, sodium citrate, sodium phosphate monobasic monohydrate, sodium hydroxide, polysorbate 80, cell culture media, and trace amounts of fetal bovine serum. RotaTeq® contains no preservatives.

IU is determined in vitro using a multivalent-quantitative polymerase chain reaction-based potency assay (M-QPA), as described in Example 2, Ranheim et al. (2006), J. Virol. Methods 131:193-201, and the vaccine reference standard developed by the manufacturer from clinical or process validation bulk vaccine lots. Live reassortant rotavirus vaccine potency can also be measured in plaque-forming units (PFU), which is determined in vitro using the standard plaque assay, and which is used to initially define the potency of the vaccine reference standard used in the M-QPA assay.

In certain embodiments, the rotavirus is live attenuated rotavirus. In certain embodiments, the invention relates to any one of the formulations described herein, wherein the live attenuated rotavirus is one or more of the several strains of rotavirus, including a G1P1A[8] human rotavirus.

In certain embodiments, the invention relates to any one of the formulations described herein, wherein the formulation comprises Rotarix® (Rotavirus Vaccine, Live, Oral, produced by Merck & Co.) or an equivalent thereof. Rotarix® is available as either a lyophilized vaccine accompanied by a liquid diluent or as a liquid suspension. In both cases the vaccine is administered orally and is indicated for prevention of rotaviral gastroenteritis caused by G1 and non-G1 (e.g., G3, G4, G9) types of rotavirus.

Each dose (1 mL after reconstitution in diluent) of lyophilized Rotarix® live attenuated rotavirus vaccine is formulated to contain at least 10⁶ median Cell Culture Infective Dose (CCID₅₀) of live, attenuated human rotavirus derived from the 89-12 strain, which belongs to the G1P1A[8] type, by propagation in Vero cells. The lyophilized vaccine contains amino acids, dextran, Dulbecco's Modified Eagle Medium (DMEM), sorbitol, and sucrose. DMEM contains the following ingredients: sodium chloride, potassium chloride, magnesium sulfate, ferric (III) nitrate, sodium phosphate, sodium pyruvate, D-glucose, concentrated vitamin solution, L-cysteine, L-tyrosine, amino acids solution, L-glutamine, calcium chloride, sodium hydrogenocarbonate, and phenol red. The liquid diluent contains calcium carbonate, sterile water, and xanthan. The diluent includes an antacid component (calcium carbonate) to protect the vaccine during passage through the stomach and prevent its inactivation due to the acidic environment of the stomach.

Each dose (1.5 mL) of liquid Rotarix® live attenuated rotavirus vaccine is also formulated to contain at least 10⁶ median Cell Culture Infective Dose (CCID₅₀) of live, attenuated human rotavirus derived from the 89-12 strain, which belongs to the G1P1A[8] type, by propagation in Vero cells. The vaccine also contains sucrose, di-sodium adipate, Dulbecco's Modified Eagle Medium (as described above), and sterile water. The vaccine also includes an antacid component to protect the vaccine during passage through the stomach and prevent its inactivation due to the acidic environment of the stomach.

In certain embodiments, the invention relates to any one of the formulations described herein, wherein the inactivated virus is present in the formulation in an amount of between about 0.001 and about 20 standard doses (as defined herein). In certain embodiments, the invention relates to formulations including one or more of the following: a type G1 human reassortant rotavirus in an amount between about 2.2×10³ and 4.4×10⁷ IU, a type G2 human reassortant rotavirus is present in an amount of between about 2.8×10³ and 5.6×10⁷ IU, a type G3 human reassortant rotavirus is present in an amount of between about 2.2×10³ and 4.4×10⁷ IU, a type G4 human reassortant rotavirus is present in an amount of between about 2.0×10³ and 4.0×10⁷ IU, and/or a type P1A[8] human reassortant rotavirus is present in an amount of between about 2.3×10³ and 4.6×10⁷ IU. In certain embodiments, the invention relates to any one of the formulations described herein, wherein a live attenuated human rotavirus is present in an amount of between 10³ and 2×10⁷ mean Cell Culture Infectious Dose (CCID₅₀).

In some embodiments, the rotavirus immunogen is one or more of the following: between 2.2×10³ and 4.4×10⁷ IU of a type G1 strain, between 2.8×10³ and 5.6×10⁷ IU of a type G2 strain, between 2.2×10³ and 4.4×10⁷ IU of a type G3 strain, between 2.0×10³ and 4.0×10⁷ IU of a type G4 strain, between 2.0×10³ and 5.6×10⁷ IU of a type G9 strain, between 2.0×10³ and 5.6×10⁷ IU of a type P[4] strain, between 2.0×10³ and 5.6×10⁷ IU of a type P[6] strain, and/or between 2.3×10³ and 4.6×10⁷ IU of a type P[8] strain.

In some embodiments, the rotavirus immunogen is one or more of the following: between 10³ and 2×10⁷ CCID₅₀ of a type G1 strain, between 10³ and 2×10⁷ CCID₅₀ of a type G2 strain, between 10³ and 2×10⁷ CCID₅₀ of a type G3 strain, between 10³ and 2×10⁷ CCID₅₀ of a type G4 strain, between 10³ and 2×10⁷ CCID₅₀ of a type G9 strain, between 10³ and 2×10⁷ CCID₅₀ of a type P[4] strain, between 10³ and 2×10⁷ CCID₅₀ of a type P[6] strain, and/or between 10³ and 2×10⁷ CCID₅₀ of a type P[8] strain.

Although some formulations will be prepared for a single use to vaccinate a single individual, other formulations comprising many standard doses may be prepared for repeated vaccinations of a single individual, or single (or repeated) vaccinations of multiple individuals (e.g., groups of individuals at a school or in a village).

Any vaccine products approved by national or regional regulatory authorities (e.g., U.S. FDA or EMEA) for treating or preventing a rotavirusinfection can be included in the formulations described herein.

Protein Stabilizers for Rotavirus Vaccines

The vaccine preparations of the invention include at least one protein stabilizer which aids in retaining the bioactivity of the vaccine antigens. In some embodiments, the protein stabilizer is selected from the group consisting silk fibroin, gelatin and albumin. In some embodiments, the protein stabilizer is silk fibroin.

In certain embodiments, the invention relates to any one of the formulations described herein, wherein the amount of protein chosen from silk fibroin, gelatin, and albumin present in the formulation immediately before drying is from 0.01% to 10% (w/v). In certain embodiments, the invention relates to any one of the formulations described herein, wherein the amount of protein chosen from silk fibroin, gelatin, and albumin in the formulation is from about 2 milligrams to about 3.2 grams per standard dose. In certain embodiments, the invention relates to any one of the formulations described herein, wherein the amount of protein chosen from silk fibroin, gelatin, and albumin in the formulation is from about 0.002 milligrams to about 64 grams.

Sugar and Sugar Alcohol Excipients for Rotavirus Vaccines

The vaccine preparations of the invention include at least one sugar or sugar alcohol excipient. In some embodiments, the sugar or sugar alcohol is selected from the group consisting of sucrose, trehalose, sorbitol, and glycerol. In some embodiments, the sugar or sugar alcohol is sucrose.

In certain embodiments, the invention relates to any of the formulations described herein, wherein the amount of sugar chosen from sucrose, trehalose, sorbitol, and glycerol present in the formulation immediately before drying is from 0.1% to 20% (w/v). In certain embodiments, the invention relates to any of the formulations described herein, wherein the amount of sugar chosen from sucrose, trehalose, sorbitol, and glycerol in the formulation is from about 2 milligrams to about 16 grams per standard dose. In certain embodiments, the invention relates to any of the formulations described herein, wherein the amount of sugar chosen from sucrose, trehalose, sorbitol, and glycerol in the formulation is from about 2 micrograms to about 320 grams.

Divalent Cations for Rotavirus Vaccines

The vaccine preparations of the invention include at least one divalent cation. In some embodiments, the divalent cation is selected from the group consisting of Ca²⁺, Mg²⁺, Mn²⁺, and Cu²⁺. These divalent cations are conveniently provided by including simple salts of the cations in the preparation. For example, chloride, carbonate or bicarbonate salts can conveniently be used (e.g., CaCl₂, CaCO₃, Ca(HCO₃)₂). In some embodiments, the divalent cation is Ca²⁺ and a chloride salt is used (i.e., CaCl₂).

In certain embodiments, the invention relates to any of the formulations described herein, wherein the amount of divalent cationic salt present in the formulation immediately before drying is from 0.1 mM to 1 M. In certain embodiments, the invention relates to any of the formulations described herein, wherein the amount of divalent cationic salt is from about 2.0×10⁻⁷ moles to about 3.2×10⁻³ moles per standard dose. In certain embodiments, the invention relates to any of the formulations described herein, wherein the amount of divalent cationic salt is from about 2.0×10⁻¹⁰ moles to about 0.064 moles.

Buffers for Rotavirus Vaccines

In certain embodiments, the invention relates to any of the formulations described herein, wherein the amount of buffer present in the formulation immediately before drying is from 0.1 mM to 1 M. In certain embodiments, the invention relates to any of the formulations described herein, wherein the amount of buffer is from about 2.0×10⁻⁷ moles to about 4.0×10⁻³ moles per standard dose. In certain embodiments, the invention relates to any of the formulations described herein, wherein the amount of buffer is from about 2.0×10⁻¹⁰ moles to about 0.08 moles. In certain embodiments, the invention relates to any of the formulations described herein, wherein the buffer solution is Mcllvane buffer, composed of citric acid and sodium phosphate dibasic dihydrate, or HEPES buffer, in each case at a pH of about 7.

Drying and Water Content for Rotavirus Vaccines

In certain embodiments, the invention relates to any one of the formulations described herein, wherein the formulation is an air-dried formulation. In certain embodiments, the invention relates to any one of the formulations described herein, wherein the formulation has been air-dried at a temperature of from about 2° C. to about 50° C. In certain embodiments, the invention relates to any one of the formulations described herein, wherein the formulation has been air-dried at a temperature of about 5° C., about 10° C., about 15° C., about 20° C., about 25° C., about 30° C., about 35° C., about 40° C., or about 45° C. In certain embodiments, the invention relates to any one of the formulations described herein, wherein the formulation has been air-dried at a temperature of about 23° C.

In certain embodiments, the invention relates to any one of the formulations described herein, wherein the formulation is vacuum-dried. Such vacuum drying can be conducted over an extended period of time (e.g., 6-12 hours), at reduced pressures (e.g., 25-100 mTorr), and at varying temperatures (e.g., −10° C. to 40° C.), with lower pressures and higher temperatures reducing drying time.

In certain embodiments, the invention relates to any one of the formulations described herein, wherein the formulation is in the form of a lyophilized powder. For example, in some specific embodiments, the formulation is lyophilized by (1) freezing at about −50° C. and holding for about 1-6 hours, followed by (2) sublimation (primary drying) at about −50 to 25° C. for about 1-96 hours under vacuum (˜25-100 milliTorr), and, optionally, (3) desorption (secondary drying) at 4 to 35° C. for about 0-24 hours under vacuum (˜25-100 milliTorr). Those of skill in the art can adjust drying times, pressures, and temperatures for best results or mere convenience.

In certain embodiments, the invention relates to any one of the formulations described herein, wherein the formulation is in the form of a film, for example, an air-dried film.

In certain embodiments, the invention relates to any one of the formulations described herein, wherein the formulation comprises 0% to 5% by mass water. These formulations with low water content (i.e., less than 5%) are most typically produced by lyophilization, but can be produced by air-drying or vacuum-drying. In certain embodiments, the invention relates to any one of the formulations described herein, wherein the formulation comprises water in an amount less than 5% by mass. In certain embodiments, the invention relates to any one of the formulations described herein, wherein the formulation comprises water in an amount less than 4% by mass. In certain embodiments, the invention relates to any one of the formulations described herein, wherein the formulation comprises water in an amount less than 3% by mass. In certain embodiments, the invention relates to any one of the formulations described herein, wherein the formulation comprises water in an amount less than 2% by mass. In certain embodiments, the invention relates to any one of the formulations described herein, wherein the formulation comprises water in an amount less than 1% by mass. In certain embodiments, the invention relates to any one of the formulations described herein, wherein the formulation comprises water in an amount less than 0.5% by mass.

In certain embodiments, the invention relates to any one of the formulations described herein, wherein the formulation comprises water in an amount between 5% and 20%. These formulations with higher water content (i.e., 5%-20%) are preferably produced by air-drying, but can be produced by vacuum-drying or partial lyophilization. Thus, in certain embodiments, the formulations comprise greater than 5%, greater than 6%, greater than 7%, greater than 8%, greater than 9%, greater than 10%, greater than 11%, greater than 12%, greater than 13%, greater than 14%, greater than 15%, greater than 16%, greater than 17%, greater than 18%, or greater than 19%, but in each case less than 20% by mass.

Stability and Bioactivity for Rotavirus Vaccines

In certain embodiments, the invention relates to any one of the formulations described herein, wherein the formulation retains at least about 70% of its original bioactivity after storage at about 25° C. for about 2 weeks. In certain embodiments, the invention relates to any one of the formulations described herein, wherein the formulation retains at least about 80% of its original bioactivity after storage at about 25° C. for about 2 weeks. In certain embodiments, the invention relates to any one of the formulations described herein, wherein the formulation retains at least about 90% of its original bioactivity after storage at about 25° C. for about 2 weeks.

In certain embodiments, the invention relates to any one of the formulations described herein, wherein the formulation retains at least about 70% of its original bioactivity after storage at about 25° C. for about 4 weeks. In certain embodiments, the invention relates to any one of the formulations described herein, wherein the formulation retains at least about 80% of its original bioactivity after storage at about 25° C. for about 4 weeks. In certain embodiments, the invention relates to any one of the formulations described herein, wherein the formulation retains at least about 90% of its original bioactivity after storage at about 25° C. for about 4 weeks.

In certain embodiments, the invention relates to any one of the formulations described herein, wherein the formulation retains at least about 70% of its original bioactivity after storage at about 25° C. for about 8 weeks. In certain embodiments, the invention relates to any one of the formulations described herein, wherein the formulation retains at least about 80% of its original bioactivity after storage at about 25° C. for about 8 weeks. In certain embodiments, the invention relates to any one of the formulations described herein, wherein the formulation retains at least about 90% of its original bioactivity after storage at about 25° C. for about 8 weeks.

In certain embodiments, the invention relates to any one of the formulations described herein, wherein the formulation retains at least about 70% of its original bioactivity after storage at about 25° C. for about 12 weeks. In certain embodiments, the invention relates to any one of the formulations described herein, wherein the formulation retains at least about 80% of its original bioactivity after storage at about 25° C. for about 12 weeks. In certain embodiments, the invention relates to any one of the formulations described herein, wherein the formulation retains at least about 90% of its original bioactivity after storage at about 25° C. for about 12 weeks.

In certain embodiments, the invention relates to any one of the formulations described herein, wherein the formulation retains at least about 60% of its original bioactivity after storage at about 37° C. for about 2 weeks. In certain embodiments, the invention relates to any one of the formulations described herein, wherein the formulation retains at least about 70% of its original bioactivity after storage at about 37° C. for about 2 weeks. In certain embodiments, the invention relates to any one of the formulations described herein, wherein the formulation retains at least about 80% of its original bioactivity after storage at about 37° C. for about 2 weeks.

In certain embodiments, the invention relates to any one of the formulations described herein, wherein the formulation retains at least about 60% of its original bioactivity after storage at about 37° C. for about 4 weeks. In certain embodiments, the invention relates to any one of the formulations described herein, wherein the formulation retains at least about 70% of its original bioactivity after storage at about 37° C. for about 4 weeks. In certain embodiments, the invention relates to any one of the formulations described herein, wherein the formulation maintains at least about 80% of its original bioactivity after storage at about 37° C. for about 4 weeks.

In certain embodiments, the invention relates to any one of the formulations described herein, wherein the formulation retains at least about 50% of its original bioactivity after storage at about 37° C. for about 8 weeks. In certain embodiments, the invention relates to any one of the formulations described herein, wherein the formulation retains at least about 60% of its original bioactivity after storage at about 37° C. for about 8 weeks. In certain embodiments, the invention relates to any one of the formulations described herein, wherein the formulation maintains at least about 70% of its original bioactivity after storage at about 37° C. for about 8 weeks.

In certain embodiments, the invention relates to any one of the formulations described herein, wherein the formulation maintains at least about 30% of its original bioactivity after storage at about 37° C. for about 12 weeks. In certain embodiments, the invention relates to any one of the formulations described herein, wherein the formulation retains at least about 40% of its original bioactivity after storage at about 37° C. for about 12 weeks. In certain embodiments, the invention relates to any one of the formulations described herein, wherein the formulation retains at least about 50% of its original bioactivity after storage at about 37° C. for about 12 weeks.

In certain embodiments, the invention relates to any one of the formulations described herein, wherein the formulation retains at least about 50% of its original bioactivity after storage at about 45° C. for about 2 weeks. In certain embodiments, the invention relates to any one of the formulations described herein, wherein the formulation retains at least about 60% of its original bioactivity after storage at about 45° C. for about 2 weeks. In certain embodiments, the invention relates to any one of the formulations described herein, wherein the formulation retains at least about 70% of its original bioactivity after storage at about 45° C. for about 2 weeks.

In certain embodiments, the invention relates to any one of the formulations described herein, wherein the formulation retains at least about 30% of its original bioactivity after storage at about 45° C. for about 4 weeks. In certain embodiments, the invention relates to any one of the formulations described herein, wherein the formulation retains at least about 40% of its original bioactivity after storage at about 45° C. for about 4 weeks. In certain embodiments, the invention relates to any one of the formulations described herein, wherein the formulation maintains at least about 50% of its original bioactivity after storage at about 45° C. for about 4 weeks.

In certain embodiments, the invention relates to any one of the formulations described herein, wherein the formulation retains at least about 30% of its original bioactivity after storage at about 45° C. for about 8 weeks. In certain embodiments, the invention relates to any one of the formulations described herein, wherein the formulation retains at least about 40% of its original bioactivity after storage at about 45° C. for about 8 weeks. In certain embodiments, the invention relates to any one of the formulations described herein, wherein the formulation retains at least about 50% of its original bioactivity after storage at about 45° C. for about 8 weeks.

In certain embodiments, the invention relates to any one of the formulations described herein, wherein the formulation retains at least about 30% of its original bioactivity after storage at about 45° C. for about 12 weeks. In certain embodiments, the invention relates to any one of the formulations described herein, wherein the formulation retains at least about 40% of its original bioactivity after storage at about 45° C. for about 12 weeks. In certain embodiments, the invention relates to any one of the formulations described herein, wherein the formulation retains at least about 50% of its original bioactivity after storage at about 45° C. for about 12 weeks.

Reconstitution and Administration of Rotavirus Vaccines

In some embodiments, the formulations described herein can be reconstituted in a pharmaceutically acceptable carrier for oral or parenteral administration (e.g., subcutaneous or intramuscular injection). As used herein, the term “pharmaceutically acceptable carrier” refers to any and all solvents, diluents, excipients, dispersion media and the like, which can be used to reconstitute a liquid dosage form. Pharmaceutically acceptable carriers useful in the invention include, but are not limited to, (x) glycols, such as propylene glycol; (xi) polyols, such as glycerin, sorbitol, mannitol and polyethylene glycol (PEG); (xii) esters, such as ethyl oleate and ethyl laurate; (xiii) agar; (xiv) buffering agents, such as magnesium hydroxide and aluminum hydroxide; (xv) alginic acid; (xvi) pyrogen-free water; (xvii) isotonic saline; (xviii) Ringer's solution; (xix) ethyl alcohol; (xx) pH buffered solutions; and oils, such as peanut oil, cottonseed oil, safflower oil, sesame oil, olive oil, corn oil and soybean oil, and other non-toxic compatible substances employed in pharmaceutical formulations.

When administering parenterally, a formulation described herein can be generally reconstituted in a unit dosage injectable form (solution, suspension, emulsion). The formulations suitable for injection include sterile aqueous solutions or dispersions. The carrier can be a solvent or dispersing medium containing, for example, water, cell culture medium, buffers (e.g., phosphate buffered saline (PBS)), polyol (for example, glycerol, propylene glycol, liquid polyethylene glycol, and the like), suitable mixtures thereof. In some embodiments, the pharmaceutical carrier can be a buffered solution (e.g., PBS).

The formulations can also contain auxiliary substances such as wetting or emulsifying agents, pH buffering agents, gelling or viscosity enhancing additives, preservatives, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation. With respect to formulations described herein, however, any vehicle, diluent, or additive used should have to be biocompatible with the antigens described herein. Those skilled in the art will recognize that the components of the formulations should be selected to be biocompatible with respect to the antigen. This will present no problem to those skilled in chemical and pharmaceutical principles, or problems can be readily avoided by reference to standard texts or by simple experiments (not involving undue experimentation).

In certain embodiments, the invention relates to a substantially dried vaccine formulation comprising, consisting essentially of, or consisting of a rotavirus, a protein, a sugar or a sugar alcohol, a divalent cation salt, a buffer salt, amino acids, dextran, and Dulbecco's Modified Eagle Medium (DMEM), wherein the protein is selected from silk fibroin, gelatin, and albumin; the sugar or the sugar alcohol is selected from sucrose, trehalose, sorbitol, and glycerol, or combinations thereof; and the buffer salt is HEPES or CP.

In certain embodiments, the invention relates to a substantially dried vaccine formulation comprising, consisting essentially of, or consisting of a live attenuated rotavirus, a protein, a sugar or a sugar alcohol, a divalent cation salt, a buffer salt, amino acids, dextran, and Dulbecco's Modified Eagle Medium (DMEM), wherein the protein is selected from silk fibroin, gelatin, and albumin; the sugar or the sugar alcohol is selected from sucrose, trehalose, sorbitol, and glycerol, or combinations thereof; and the buffer salt is HEPES or CP.

In certain embodiments, the invention relates to a substantially dried vaccine formulation comprising, consisting essentially of, or consisting of a live reassortant rotavirus, a protein, a sugar or a sugar alcohol, a divalent cation salt, a buffer salt, amino acids, dextran, and Dulbecco's Modified Eagle Medium (DMEM), wherein the protein is selected from silk fibroin, gelatin, and albumin; the sugar or the sugar alcohol is selected from sucrose, trehalose, sorbitol, and glycerol, or combinations thereof; and the buffer salt is HEPES or CP.

In certain embodiments, the invention relates to a substantially dried vaccine formulation comprising, consisting essentially of, or consisting of a rotavirus, a protein, a sugar or a sugar alcohol, a divalent cation salt, a buffer salt, amino acids, dextran, and Dulbecco's Modified Eagle Medium (DMEM), wherein the protein is silk fibroin; the sugar or the sugar alcohol is sucrose; the divalent cation salt is calcium chloride; and the buffer salt is HEPES or CP.

In certain embodiments, the invention relates to a substantially dried vaccine formulation comprising, consisting essentially of, or consisting of a live attenuated rotavirus, a protein, a sugar or a sugar alcohol, a divalent cation salt, a buffer salt, amino acids, dextran, and Dulbecco's Modified Eagle Medium (DMEM), wherein the protein is silk fibroin; the sugar or the sugar alcohol is sucrose; the divalent cation salt is calcium chloride; and the buffer salt is HEPES or CP.

In certain embodiments, the invention relates to a substantially dried vaccine formulation comprising, consisting essentially of, or consisting of a live reassortant rotavirus, a protein, a sugar or a sugar alcohol, a divalent cation salt, a buffer salt, amino acids, dextran, and Dulbecco's Modified Eagle Medium (DMEM), wherein the protein is silk fibroin; the sugar or the sugar alcohol is sucrose; the divalent cation salt is calcium chloride; and the buffer salt is HEPES or CP.

Exemplary Methods for Preparing Formulations of Rotavirus Vaccines

In some embodiments, the invention relates to a method of preparing any one of the formulations described herein, comprising the steps of:

mixing; and

lyophilizing or drying the vaccine mixture, thereby forming a substantially dried vaccine mixture.

In some embodiments, the invention relates to any one of the methods described herein, wherein the vaccine mixture is lyophilized. In some embodiments, the invention relates to any one of the methods described herein, wherein the vaccine mixture is lyophilized to form a substantially dried vaccine mixture in the form of a powder.

In some embodiments, the invention relates to any one of the methods described herein, wherein the vaccine mixture is substantially dried, for example, air-dried. In some embodiments, the invention relates to any one of the methods described herein, wherein the vaccine mixture is air-dried to form a substantially dried vaccine mixture in the form of a film.

In some embodiments, the invention relates to any one of the methods described herein, further comprising the step of:

mixing the substantially dried vaccine mixture with a diluent.

In some embodiments, the invention relates to any one of the methods described herein, wherein the concentration of protein in solution prior to drying is between about 0.1 and 10% w/v.

In some embodiments, the invention relates to any one of the methods described herein, wherein the protein is silk fibroin. In some embodiments, the invention relates to any one of the methods described herein, wherein the silk fibroin solution does not comprise sericin. In some embodiments, the invention relates to any one of the methods described herein, wherein the silk fibroin solution does not comprise a salt.

In some embodiments, the invention relates to any one of the methods described herein, further comprising the step of:

preparing the silk fibroin solution from a sample comprising a cocoon from a silkworm Bombyx mori.

The aqueous silk fibroin solution can be prepared using techniques known in the art. Suitable processes for preparing silk fibroin solutions are disclosed, for example, in U.S. Pat. No. 7,635,755; WO 2005/012606; and WO 2008/127401.

In accordance with the conventional practice, the formulations described herein are desirably processed under aseptic conditions using components which preliminarily have been rendered bacterially sterile. Sterility on storage can be maintained by incorporation of an antigen-compatible germicidal substance such as thimerosal.

Exemplary Methods of Using Formulations of Rotavirus Vaccines

In certain embodiments, the invention relates to a method of treating or preventing an infection caused by a rotavirus, comprising the step of:

administering to a subject in need thereof a therapeutically or prophylactically effective amount or dose of any one of the formulations described herein, thereby eliciting an immune response in the subject and treating or preventing the infection.

In certain embodiments, the invention relates to any one of the methods described herein, wherein the subject is a mammal.

In certain embodiments, the invention relates to any one of the methods described herein, wherein the subject is a mammal susceptible to or suffering from an infection caused by a rotavirus.

In certain embodiments, the invention relates to any one of the methods described herein, wherein the subject is a human.

In certain embodiments, the invention relates to any one of the methods described herein, wherein the subject is a human under the age of five.

In some embodiments, the invention relates to any one of the methods described herein, wherein the formulation is administered to the subject in two or three spaced doses.

In some embodiments, the invention relates to any one of the methods described herein, wherein the formulation is administered to the subject in three spaced doses. For example, the first dose is administered when the subject is from about 6 weeks to about 12 weeks of age, and the second and third doses are administered at 4- to 10-week intervals.

In some embodiments, the invention relates to any one of the methods described herein, wherein the formulation is administered to the subject in two spaced doses. For example, the first dose is administered when the subject is from about 6 weeks to about 20 weeks of age, and the second dose is administered at least 4 weeks after the first dose.

In certain embodiments, the invention relates to any one of the methods described herein, wherein the formulation is in the form of a film.

In certain embodiments, the invention relates to any one of the methods described herein, wherein the formulation is in the form of a powder.

In some embodiments, the invention relates to any one of the methods described herein, wherein the formulation is administered to the subject orally.

In certain embodiments, the invention relates to any one of the methods described herein, wherein the formulation is in the form of a film, further comprising the step of: mixing the formulation with a diluent prior to administering to the subject.

In certain embodiments, the invention relates to any one of the methods described herein, wherein the formulation is in the form of a powder, further comprising the step of: mixing the formulation with a diluent prior to administering to the subject.

In some embodiments, the invention relates to any one of the methods described herein, wherein the formulation is administered to the subject by injection, such as subcutaneous, dermal (e.g., transdermal, intradermal or interdermal), or intramuscular injection.

### Exemplary Kits and Devices

In certain embodiments, the invention relates to a package or kit comprising any one of the formulations described herein (e.g., a formulation including an immunogen as described herein, such as an enterovirus, a flavivirus, a rotavirus, a measles virus, a mumps virus, a rubella virus, or an influenza virus). The packages can be prepared in various types of containers, which can be selected from the group consisting of a vial, an ampule, a capsule, a tube, a delivery device, a bottle, and a packet. In some embodiments, the delivery device is a syringe. In some embodiments, the syringe can be needleless. The formulation contained in a package can be in a form of a hydrogel, gel-like particles, powder, microspheres, nanospheres, or any combinations thereof. In some embodiments, the formulation contained in a package can be lyophilized. In some embodiments, the formulation can be loaded in a syringe for injection.

Kits provided herein comprise a package described herein, and a pharmaceutically acceptable solution, e.g., PBS. In some embodiments, the kits can further comprise at least one delivery device for administering a formulation described herein to a subject. In other embodiments, the kits can further comprise a disinfectant. In certain embodiments, such packages, and kits described herein can be used for vaccination purposes.

Delivery devices pre-loaded with at least one formulation described herein are also within the scope of various aspects described herein. Embodiments of a delivery device comprises at least one chamber with an outlet, wherein the at least one chamber comprises a pre-determined amount of the formulation described herein, and the outlet provides an exit for the formulation.

The term “chamber” as used herein refers to any structure configured to store and/or convey a formulation described herein. The chamber can be of any shape or any size, depending on users' applications, needs, and/or preferences. An exemplary chamber includes, but is not limited to, a barrel, a tube, a cassette, and a depression, e.g., a microwell.

Examples of delivery devices described herein include, but are not limited to, a syringe, a dry powder injector, a nasal spray, a nebulizer, and an implant. In some embodiments, an implant can be a microchip, e.g., the ones described in U.S. Pat. Nos. 5,797,898; 6,669,683; 7,052,488; and 7,582,080. In some embodiments, the delivery devices can be used for vaccination. In such embodiments, vaccine delivery devices/systems can include, but are not limited to, the ones described in US 2004/0133160; US 2004/0096455; US 2005/0112135; US 2005/0123565; US 2009/0043280; and US 2009/0143724, as well as U.S. Pat. Nos. 5,346,481; and 5,900,238.

### EXAMPLES

The invention now being generally described, it will be more readily understood by reference to the following examples, which are included merely for purposes of illustration of certain aspects and embodiments of the present invention, and are not intended to limit the invention.

### Example 1—Purification of Silk Fibroin

Silk fibroin solution was prepared according to established methods. Briefly, pieces of cocoons from the silkworm Bombyx mori were first boiled in 0.02 M Na₂CO₃ for 60 or 180 minutes to remove sericin protein which is present in unprocessed, natural silk. A 180 minute boiling time was used in the preparation of the enterovirus formulations below. A 60 minute boiling time was used in the preparation of the rotavirus formulations below. A 180 minute boiling time was used in the preparation of the enterovirus formulations below. A 180 minute boiling time was used in the preparation of the dried flavivirus formulations below. A 60 minute boiling time was used in the preparation of the liquid flavivirus formulations below. After rinsing three times in ultrapure water and air-drying overnight, fibroin fibers were solubilized in 9.3 M LiBr at 60° C. for 4 hours to produce a solution comprising the constituent silk fibroin proteins. This solution was then dialyzed against ultrapure water for 48 hours to remove salt and centrifuged for 20 minutes at 4° C. (9,000 rpm) twice. This process resulted in an aqueous silk fibroin solution of roughly 6-7% wt/vol, which was sterile-filtered prior to use.

### Examples Related to Enterovirus Vaccine Formulations

### Example 2—Preparation of Dried IPV Formulations

### Vaccine Dialysis

Polio vaccine (IPOL®; Sanofi-Pasteur) was purchased from Henry-Schein and was dialyzed against 10 mM citrate-phosphate buffer (pH 7.4) to substantially remove commercial excipients (2-phenoxyethanol, formaldehyde) prior to formulation. Briefly, each liter of dialysis buffer was prepared by mixing 0.3203 g of citric acid (Sigma-Aldrich) with 1.483 g of sodium phosphate dibasic dihydrate (Sigma-Aldrich) in 1.0 L of Milli-Q water. This buffer was pre-cooled overnight at 4° C. Vaccine was loaded into either 3-mL or 12-mL dialysis cassettes (Slide-A-Lyzer 3.5 kDa; Thermo-Fisher) depending on needed volume and dialyzed against 1-L or 2-L of buffer respectively. The dialysis process was performed in a cold room (4° C.) for 24 hours, with buffer replacement at 2, 4, 6, and 22 hours. The dialyzed vaccine was then recovered from cassettes and refrigerated prior to formulation.

### IPV Bioactivity Assay

Analysis of poliovirus D-antigen content for Types 1, 2, and 3, was performed according to an ELISA protocol developed by the CDC polio and picornavirus laboratory (Edens et al. (2015), supra). Briefly, ELISA plates (Immulon 2HB, Thermo Scientific) were coated overnight at 4° C. using capture antibodies (Anti-polio 1 [14D2 (7C5)], Novus Biologicals; Anti-polio 2 [24E2], Enzo Life Sciences; Anti-polio 3 [clone 4D5], Fisher Scientific) diluted 1:500 for Types 1 and 3 and 1:1,000 for type 2 in 50 mM Carbonate-Bicarbonate Buffer (pH 9.6, Sigma Aldrich). Plates were then washed 4 times by adding 175 μl/well of 0.01M PBS+TWEEN® 20 (polysorbate 20) (0.05%) (pH 7.2, Sigma) and removing by flicking over a waste container. The plates were then blocked by adding 100 μl of Wash buffer+0.5% Gelatin (Difco)+0.25% TWEEN® 20 (polysorbate 20) (Sigma) to each well and incubating for 1 hour at 37° C. Formulated vaccine samples were then diluted 1:10 in blocking buffer and monitored for reconstitution time and appearance. Serial dilutions of vaccine were prepared as standards. After washing plates, a 50 μl volume of sample or standard was added to triplicate wells for each serotype and then stored at 37° C. for one hour before another wash step and the addition of sandwich antibody. HRP-conjugated antibodies were prepared prior to each ELISA (Lightning-Link HRP Antibody Labeling kit, Novus Biologicals) and diluted in blocking buffer. After a final incubation step (1 hour at 37° C.) the plates were washed again and 50 μl of 3,3′,5,5′-tetramethylbenzidine (TMB) substrate (KPL, Inc.) was added to each well. The plates were developed for 10 minutes at room temperature away from light and then stopped using TMB BlueSTOP solution (KPL, Inc.). The absorbance of the wells was read at 620 nm using a plate reader (Cytation 3, BioTek).

### Formulation Preparation and Drying

To prepare formulations before drying, 2× concentrated mixtures of excipients were sterile filtered using a 0.22 μm syringe filter. These excipient mixtures were then diluted 1:1 with dialyzed polio vaccine to create the final formulation. In the case of formulations that were being prepared for lyophilization, the excipient mixtures were diluted 1:1 with dialyzed polio vaccine. Lyophilization was then performed in a Virtis Genesis 25 XL Pilot Lyophilizer (SP Scientific). In the case of formulations that were being prepared for vacuum-drying, the excipient mixtures were diluted 1:1 with dialyzed polio vaccine. Vacuum-drying was then performed in a Virtis Genesis 25 XL Pilot Lyophilizer (SP Scientific). In the case of formulations that were being prepared for air-drying, the mixtures of excipients were diluted 1:1 with dialyzed polio vaccine. Formulations were then cast onto PDMS molds or into glass vials and allowed to dry, in some cases in a controlled humidity environment, and in some cases in a controlled temperature and pressure environment, such as a lyophilizer.

### Example 3—Air-Dried IPV Formulation

Exemplary IPV formulations prepared using the method described above in Example 2 contained the following excipient mixture in the following final concentration prior to air-drying:
(a) 2.4% w/v of silk fibroin, prepared by the method described above in Example 1,
(b) 5% w/v of sucrose,
(c) 10 mM of magnesium chloride, and
(d) 10 mM of citrate-phosphate buffer.

These formulations were cast onto circular molds with 12 mm diameter and dried overnight under ambient conditions (about 20-25 degrees C. and about 30-40% relative humidity). Each mold held 0.1 mL total of vaccine formulation before drying.

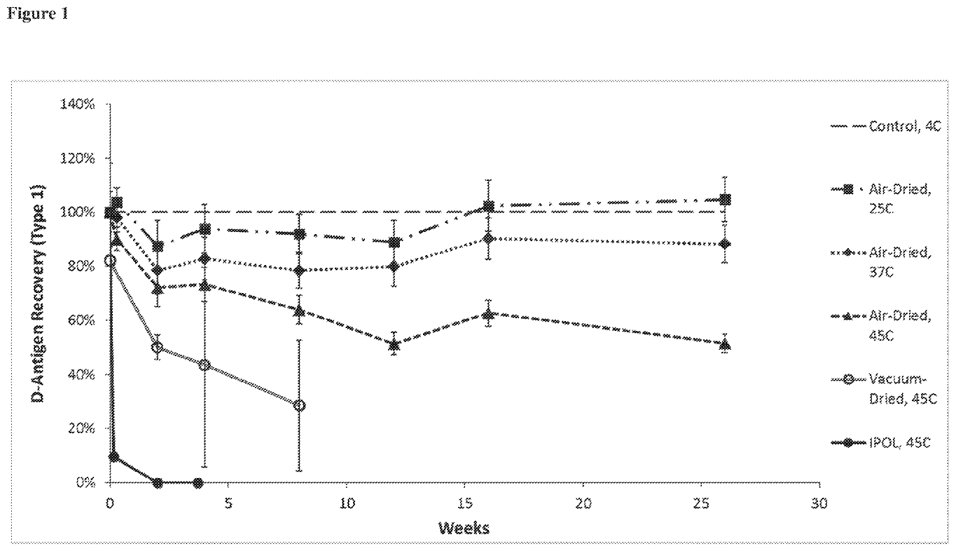

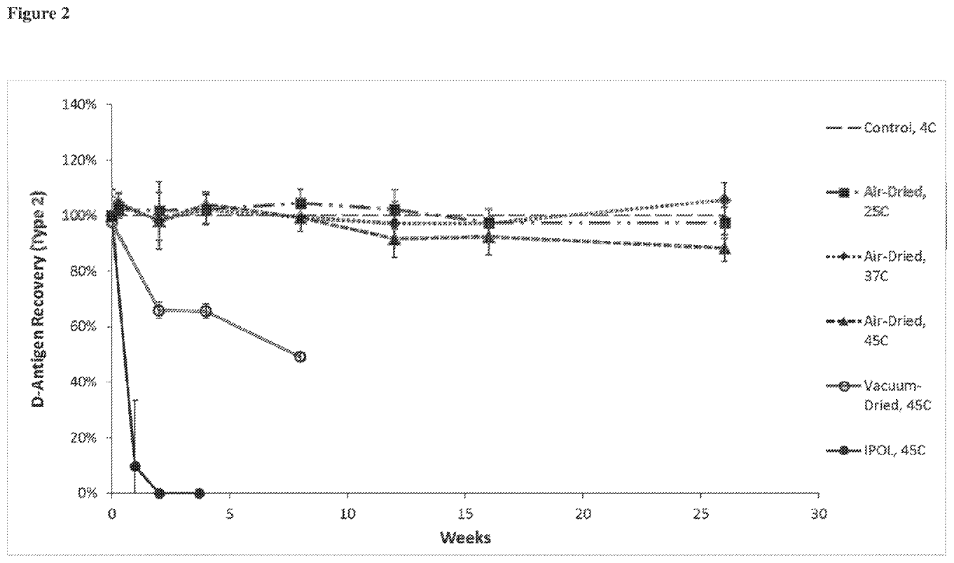

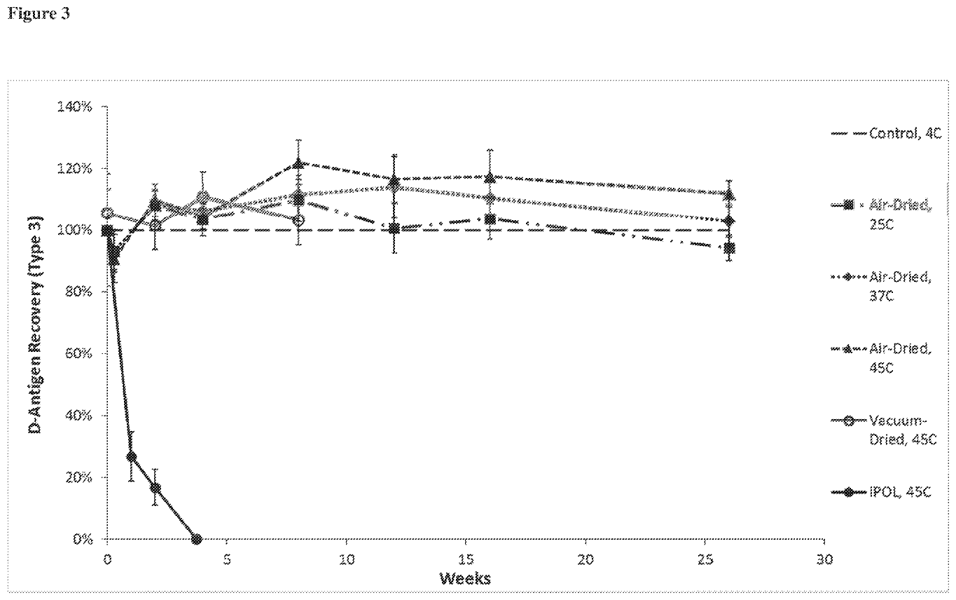

Films (n=3 per temperature and timepoint) were placed on stability at 4, 25, 37, and 45 degrees Celsius. Potency was evaluated using a D-antigen ELISA at 0, 2, 4, 8, 12, 16, and 26 weeks. The stability results are depicted in FIGS. 1, 2, and 3.

### Example 4—Vacuum-Dried IPV Formulation

Exemplary IPV formulations prepared using the method described above in Example 2 contained the following excipient mixture in the following final concentration prior to vacuum-drying:
(a) 2.4% w/v of silk fibroin, prepared by the method described above in Example 1,
(b) 5% w/v of sucrose,
(c) 10 mM of magnesium chloride, and
(d) 10 mM of citrate-phosphate buffer.

These formulations were dried in a Virtis Genesis 25 XL Pilot Lyophilizer (SP Scientific) using the following vacuum-drying cycle:

| Temperature (° C.) | Pressure (mT) | Time (minutes) |
|---|---|---|
| 15 | 900 | 30 |
| 15 | 750 | 30 |
| −5 | 50 | 60 |
| 10 | 50 | 120 |
| 20 | 50 | 120 |
| 30 | 50 | 120 |

Vials (n=3 per temperature and timepoint) were placed on stability at 45 degrees Celsius. Potency was evaluated using a D-antigen ELISA at 0, 2, 4, and 8 weeks. The stability results are depicted in FIGS. 1, 2, and 3.

### Example 5—Air-Dried IPV Formulation

Exemplary IPV formulations prepared using the method described above in Example 2 contained the following excipient mixture in the following final concentration prior to air-drying:
(a) 2.4% w/v of silk fibroin, prepared by the method described above in Example 1,
(b) 2.4% w/v of trehalose,
(c) 10 mM of magnesium chloride, and
(d) 10 mM of citrate-phosphate buffer.

These formulations were cast onto circular molds with 12 mm diameter and dried overnight under ambient conditions (about 20-25 degrees C. and about 30-40% relative humidity). Each mold held 0.1 mL total of vaccine formulation before drying.

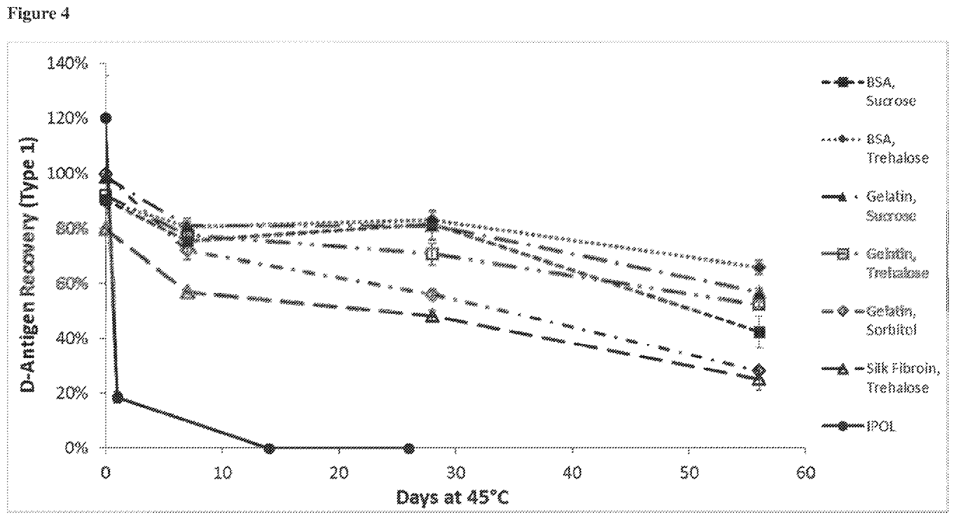

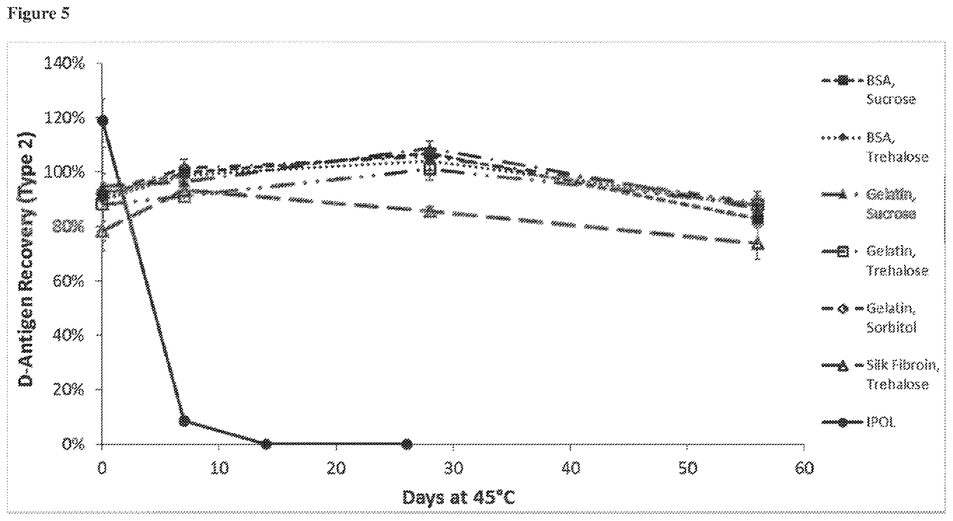

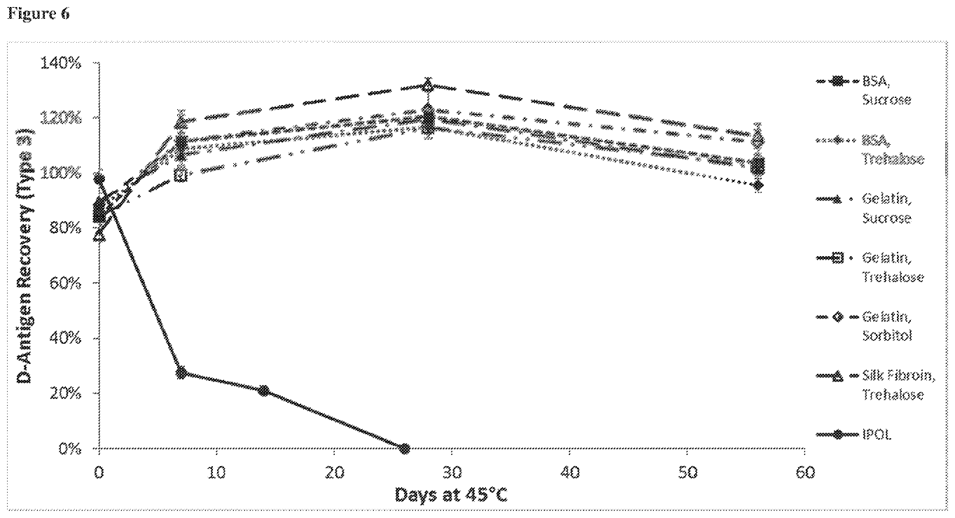

Films (n=3 per temperature and timepoint) were placed on stability at 45 degrees Celsius. Potency was evaluated using a D-antigen ELISA at 0, 7, 28, and 56 days. The stability results are depicted in FIGS. 4, 5, and 6.

### Example 6—Air-Dried IPV Formulation

Exemplary IPV formulations prepared using the method described above in Example 2 contained the following excipient mixture in the following final concentration prior to air-drying:
(a) 2.4% w/v of bovine serum albumin,
(b) 2.4% w/v of sucrose,
(c) 10 mM of magnesium chloride, and
(d) 10 mM of citrate-phosphate buffer.

These formulations were cast onto circular molds with 12 mm diameter and dried overnight under ambient conditions (about 20-25 degrees C. and about 30-40% relative humidity). Each mold held 0.1 mL total of vaccine formulation before drying.

Films (n=3 per temperature and timepoint) were placed on stability at 45 degrees Celsius. Potency was evaluated using a D-antigen ELISA at 0, 7, 28, and 56 days. The stability results are depicted in FIGS. 4, 5, and 6.

### Example 7—Air-Dried IPV Formulation

Exemplary IPV formulations prepared using the method described above in Example 2 contained the following excipient mixture in the following final concentration prior to air-drying:
(a) 2.4% w/v of bovine serum albumin,
(b) 2.4% w/v of trehalose,
(c) 10 mM of magnesium chloride, and
(d) 10 mM of citrate-phosphate buffer.

These formulations were cast onto circular molds with 12 mm diameter and dried overnight under ambient conditions (about 20-25 degrees C. and about 30-40% relative humidity). Each mold held 0.1 mL total of vaccine formulation before drying.

Films (n=3 per temperature and timepoint) were placed on stability at 45 degrees Celsius. Potency was evaluated using a D-antigen ELISA at 0, 7, 28, and 56 days. The stability results are depicted in FIGS. 4, 5, and 6.

### Example 8—Air-Dried IPV Formulation

Exemplary IPV formulations prepared using the method described above in Example 2 contained the following excipient mixture in the following final concentration prior to air-drying:
(a) 2.4% w/v of hydrolyzed gelatin,
(b) 2.4% w/v of sucrose,
(c) 10 mM of magnesium chloride, and
(d) 10 mM of citrate-phosphate buffer.

These formulations were cast onto circular molds with 12 mm diameter and dried overnight under ambient conditions (about 20-25 degrees C. and about 30-40% relative humidity). Each mold held 0.1 mL total of vaccine formulation before drying.

Films (n=3 per temperature and timepoint) were placed on stability at 45 degrees Celsius. Potency was evaluated using a D-antigen ELISA at 0, 7, 28, and 56 days. The stability results are depicted in FIGS. 4, 5, and 6.

### Example 9—Air-Dried IPV Formulation

Exemplary IPV formulations prepared using the method described above in Example 2 contained the following excipient mixture in the following final concentration prior to air-drying:
(a) 2.4% w/v of hydrolyzed gelatin,
(b) 2.4% w/v of trehalose,
(c) 10 mM of magnesium chloride, and
(d) 10 mM of citrate-phosphate buffer.

These formulations were cast onto circular molds with 12 mm diameter and dried overnight under ambient conditions (about 20-25 degrees C. and about 30-40% relative humidity). Each mold held 0.1 mL total of vaccine formulation before drying.

Films (n=3 per temperature and timepoint) were placed on stability at 45 degrees Celsius. Potency was evaluated using a D-antigen ELISA at 0, 7, 28, and 56 days. The stability results are depicted in FIGS. 4, 5, and 6.

### Example 10—Air-Dried IPV Formulation

Exemplary IPV formulations prepared using the method described above in Example 2 contained the following excipient mixture in the following final concentration prior to air-drying:
(a) 2.4% w/v of hydrolyzed gelatin,
(b) 2.4% w/v of sorbitol,
(c) 10 mM of magnesium chloride, and
(d) 10 mM of citrate-phosphate buffer.

These formulations were cast onto circular molds with 12 mm diameter and dried overnight under ambient conditions (about 20-25 degrees C. and about 30-40% relative humidity). Each mold held 0.1 mL total of vaccine formulation before drying.

Films (n=3 per temperature and timepoint) were placed on stability at 45 degrees Celsius. Potency was evaluated using a D-antigen ELISA at 0, 7, 28, and 56 days. The stability results are depicted in FIGS. 4, 5, and 6.

### Examples Related to Rotavirus Vaccine Formulations

### Example 11—Preparation of Dried Rotavirus Vaccine Formulations

### Vaccine Dialysis

RotaTeq® (Merck & Co., Inc.) was purchased from Henry Schein (Melville, N.Y.). Two different dialysis buffers were prepared and sterilized. Citrate-Phosphate buffer at pH 7.0 was prepared by mixing 10.8 mM Sodium Citrate dihydrate (JT Baker), 5.4 mM Sodium Phosphate monobasic (Sigma) and 1.7 mM Sodium Hydroxide (Sigma). HEPES buffer at pH 7 was prepared by combining 16 mM HEPES free acid (JT Baker) with 4 mM HEPES sodium salt (JT Baker). The solutions were sterile filtered using 0.22 μm sterile filter and stored overnight at 4° C. Dialysis beaker, aluminum foil and magnetic stir bar were sterilized by autoclaving. Dialysis cassettes (Slide-A-Lyzer G2, 10 KDa, 3-15 ml, gamma irradiated) were soaked in buffer for ˜2 min before filling the vaccine. 2 ml of vaccine was transferred and the dialysis was carried out overnight in a cold room. For dialysis in HEPES buffer, the dialysis buffer was changed after 2 and 5 hours whereas no buffer change was carried out for Citrate-Phosphate buffer system. All the steps were carried out under sterile conditions. The dialyzed vaccine was stored on ice immediately after dialysis during formulation.

### Vaccine De-Salting

RotaTeq® (Merck & Co., Inc.) was purchased from Henry Schein (Melville, N.Y.). Two different desalting buffers were prepared and sterilized. Citrate-Phosphate buffer at pH 7.0 was prepared by mixing 10.8 mM Sodium Citrate dihydrate (JT Baker), 5.4 mM Sodium Phosphate monobasic (Sigma) and 1.7 mM Sodium Hydroxide (Sigma). HEPES buffer at pH 7 was prepared by combining 16 mM HEPES free acid (JT Baker) with 4 mM HEPES sodium salt (JT Baker). The solutions were sterile filtered using 0.22 μm sterile filter and stored overnight at 4° C. Desalting columns, Amicon Ultra 4, 3 kDa (Millipore) were sterilized by filling with 70% ethanol and spinning for 1 minute at 3000 rcf. After removal of ethanol, columns were washed three times with sterile buffer by filling and spinning for 1 minute at 3000 rcf. 1.3 ml of stock vaccine was transferred into each column and centrifuged for 30 minutes at 3000 rcf. 2 ml of buffer was then added to the column and centrifugation repeated. This cycle was repeated another 5 times adding 3, 3, 4, 4, and 5 ml of buffer after each spin, respectively. The volume recovered after the final spin was approximately 325 μl of vaccine. This was then diluted in buffer at a 1:1 ratio to recover 650 μl of 2× concentrated vaccine. The vaccine was stored on ice immediately after desalting and concentration during formulation.

### RotaTeq® Potency Assay

Stability of RotaTeq® was measured by RT-PCR potency assay. These assays were performed using confluent monolayers of Vero cells (ATCC CCL-81, African Green Monkey kidney cell line) plated in growth media (M199/5% FBS/1% PenStrep) in 96-well plates and cultured for 4-7 days at 37° C., 5% CO₂. Serial dilutions of test samples and controls were made in infection media (high glucose DMEM/1% GlutaMAX-I/1% PenStrep) plus 0.5 μg/mL TPCK trypsin and plated on the Vero cell monolayers at 40 μLs/well. The infected 96-well plates were cultured for 21-24 hours at 37° C., 5% CO₂. The infected cell monolayers were then detergent lysed and analyzed by 1-step RT-PCR using the Cells-to-CT™ 1-Step TaqMan® Kit (Life Technologies, A25603) and Rotavirus G1 reassortant specific primers and probes. Following detergent lysis, the 96-well plates were immediately sealed and frozen at −20° C. until analyzed by RT-PCR. Samples were analyzed on a StepOnePlus Real-Time PCR System using RotaTeq® G1 specific TaqMan® Probe and Primers (Life Technologies):

0.9 μM RotaTeq®-G1 specific primer (forward) =
5′-TGTCTGTATTATCCAACTGAAGCAAGT
0.9 μM RotaTeq®-G1 specific primer (reverse) =
5′-CCCTTTGTAAGAAAACATTTGCGA
0.25 μM RotaTeq®-G1 specific 6FAM-TAMRA probe =
5′ FAM-TCAAATCAATGATGGTGACTGGAAAGACACA5-TAMRA 3′

2 μLs of cell lysate and 18 μLs of MasterMix containing the primers and probes were analyzed per RT-PCR reaction well, with the StepOnePlus Real-Time PCR System set to the following Fast cycling conditions:

| | No. of | | | |
|---|---|---|---|---|
| Step | cycles | Temp. | Time | |
| Reverse transcription | 1 | 50° C. | 5 | min |
| RT inactivation/initial | 1 | 95° C. | 20 | sec |
| denaturation | | | | |
| Amplification | 40 | 95° C. | 3 | sec |
| | | 60° C. | 30 | sec |

The data from each RT-PCR plate was processed using StepOne software and a fluorescence threshold (C_{T}) value was generated for each reaction well. Each sample's C_{T} value at a 1:100 dilution was then used as a measure of its relative level of Rotavirus potency.

### Formulation Preparation and Drying

Various formulations were prepared by combining silk fibroin protein, CaCl₂ (Sigma) and sucrose (JT Baker) from stock solutions at a final concentration of 2% (w/v), 10 mM and 5% (w/v), respectively. All the stock solutions were sterile filtered using 0.22 μm syringe filter and stored on ice before use. Samples were mixed by gentle pipetting and all formulation steps were carried out in a biosafety cabinet under sterile conditions.

For air-drying of vaccine formulations, first, 12 mm PDMS molds were prepared. For this 40 g of reagent A was mixed with 4 g of reagent B (Sylgard® 184 silicon elastomer kit, Dow Corning Inc.), ˜35 ml of mixture was spread in a large petri dish and cured overnight at 60° C. Molds were cut from the plate using a 12 mm biopsy punch and sterilized by washing with 70% ethanol followed by washing three times with the sterile water. The washed molds were dried overnight in a biosafety cabinet. For film preparation, 100 μl of the vaccine solution containing various ingredients was transferred to a PDMS mold, spread evenly using a pipette tip and left overnight at room temperature (about 20 to 26° C.) in a biosafety cabinet for drying. After drying, the films were lifted and transferred to sterile tubes, sealed with parafilm and stored at specified temperatures. In some cases the films were transferred to glass vials, filled with ultra-pure nitrogen in a freeze dryer, stoppered with chlorobutyl stoppers and sealed with aluminum seals.

All the steps were carried out under sterile conditions.

For lyophilization of vaccine formulations, 2 ml glass vials (Wheaton) and 13 mm chlorobutyl 2-leg lyophilization stoppers (Wheaton) were washed with a rinse free detergent (Micro 90, VWR), thoroughly cleaned with water, sterilized by autoclaving and dried overnight at 105° C. 200 μl aliquots of samples and controls were filled in glass vials, partially stoppered and loaded in to a Virtis Genesis 25 XL Pilot lyophilizer (SP Scientific) at a shelf temperature of 5° C. Samples were frozen by reducing the shelf temperature to −52° C. at a rate of 0.26° C./min and held at the same temperature for 180 min. Exemplary cycle parameters for the lyophilization are described in the table below. After completion of the drying, the lyophilization chamber was back filled with ultra-pure nitrogen, glass vials were stoppered and sealed with 13 mm aluminum seals (Wheaton).

Load temp 5° C.

| Thermal Treatment | Temperature, ° C. | Time, Min | Pressure, mTorr |
|---|---|---|---|
| Rate | −52 | 180 | |
| Hold | −52 | 180 | |

Primary Drying

| | | | |
|---|---|---|---|
| Hold | −52 | 60 | 55 |
| Rate | −35 | 170 | 55 |
| Hold | −35 | 1440 | 55 |
| Rate | −30 | 250 | 55 |
| Hold | −30 | 2500 | 55 |

Secondary Drying

| | | | |
|---|---|---|---|
| Rate | 25 | 500 | 55 |
| Hold | 25 | 120 | 55 |
| Storage | 5 | | 200 |

### Example 12—Lyophilized Rotavirus Formulation

An exemplary rotavirus formulation prepared using the method described above in Example 11 contained vaccine dialyzed using the method described above in Example 11 and the following excipient mixture in the following final concentration prior to lyophilization:
(a) 2.0% w/v of silk fibroin, prepared by the method described above in Example 1,
(b) 5.0% w/v of sucrose,
(c) 10 mM of calcium chloride, and
(d) 12.6 mM of HEPES buffer.

These formulations were put into 2 ml glass vials and dried by lyophilization as described in Example 11 above. Each vial held 0.2 mL total of vaccine formulation before drying.

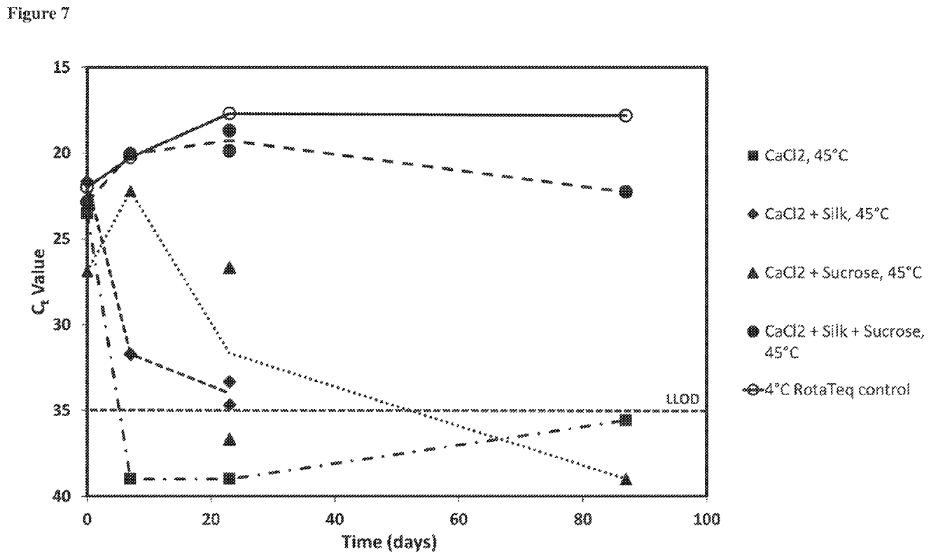

Films were placed on stability at 45° C. Potency was evaluated using RT-PCR at 0, 7, 23, and 87 days. The stability results are depicted in FIG. 7.

### Example 13—Lyophilized Rotavirus Formulation

An exemplary rotavirus formulation prepared using the method described above in Example 11 contained vaccine dialyzed using the method described above in Example 11 and the following excipient mixture in the following final concentration prior to lyophilization:
(a) 2.0% w/v of silk fibroin, prepared by the method described above in Example 1,
(b) 5.0% w/v of sucrose,
(c) 10 mM of calcium chloride, and
(d) 12.6 mM of HEPES buffer.

These formulations were put into 2 ml glass vials and dried by lyophilization as described in Example 11 above. Each vial held 0.2 mL total of vaccine formulation before drying.

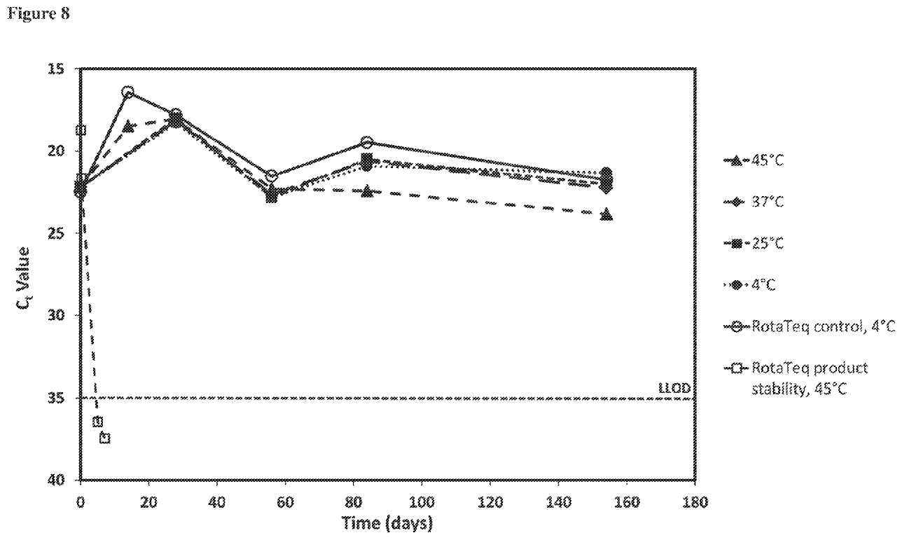

Films were placed on stability at 45° C. Potency was evaluated using RT-PCR at 0, 14, 28, 56, 84, and 154 days. The stability results are depicted in FIG. 8.

### Example 14—Lyophilized Rotavirus Formulation

An exemplary rotavirus formulation prepared using the method described above in Example 11 contained vaccine de-salted using the method described above in Example 11 and the following excipient mixture in the following final concentration prior to lyophilization:
(a) 2.0% w/v of silk fibroin, prepared by the method described above in Example 1,
(b) 5.0% w/v of sucrose,
(c) 10 mM of calcium chloride, and
(d) 9.76 mM of HEPES buffer.

These formulations were put into 2 ml glass vials and dried by lyophilization as described in Example 11 above. Each vial held 0.2 mL total of vaccine formulation before drying.

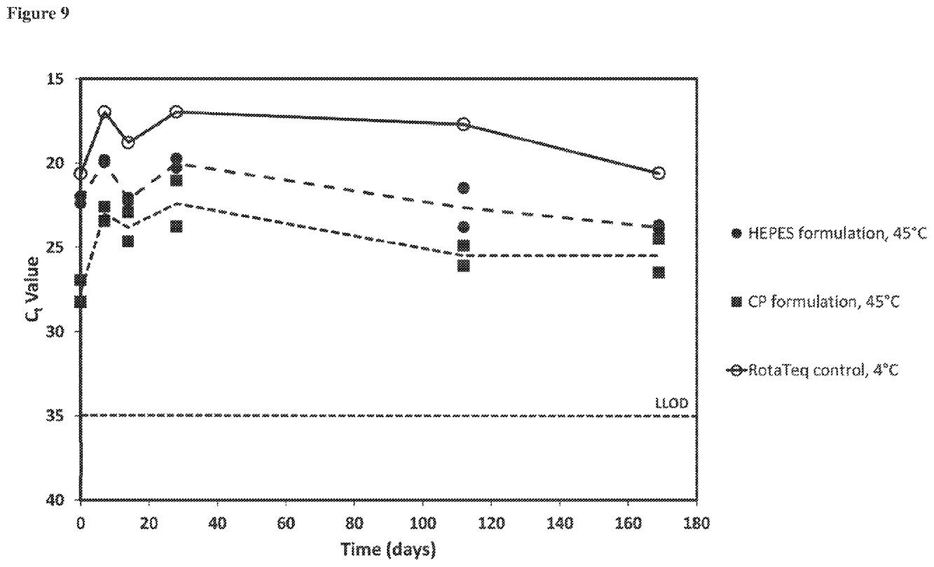

Films were placed on stability at 45° C. Potency was evaluated using RT-PCR at 0, 7, 14, 28, 112, and 169 days. The stability results are depicted in FIG. 9.

### Example 15—Lyophilized Rotavirus Formulation

An exemplary rotavirus formulation prepared using the method described above in Example 11 contained vaccine de-salted using the method described above in Example 11 and the following excipient mixture in the following final concentration prior to lyophilization:
(a) 2.0% w/v of silk fibroin, prepared by the method described above in Example 1,
(b) 5.0% w/v of sucrose,
(c) 10 mM of calcium chloride, and
(d) 9.76 mM of citrate phosphate buffer.

These formulations were put into 2 ml glass vials and dried by lyophilization as described in Example 11 above. Each vial held 0.2 mL total of vaccine formulation before drying.

Films were placed on stability at 45° C. Potency was evaluated using RT-PCR at 0, 7, 14, 28, 112, and 169 days. The stability results are depicted in FIG. 9.

### Example16—Air-Dried Rotavirus Formulation

An exemplary rotavirus formulation prepared using the method described above in Example 11 contained vaccine dialyzed using the method described above in Example 11 and the following excipient mixture in the following final concentration prior to air-drying:
(e) 2.0% w/v of silk fibroin, prepared by the method described above in Example 1,
(f) 5.0% w/v of sucrose,
(g) 10 mM of calcium chloride, and
(h) 12.6 mM of HEPES buffer.

These formulations were cast onto circular molds with 12 mm diameter and dried overnight under ambient conditions (about 20-25° C. and about 30-40% relative humidity). Each mold held 0.1 mL total of vaccine formulation before drying.

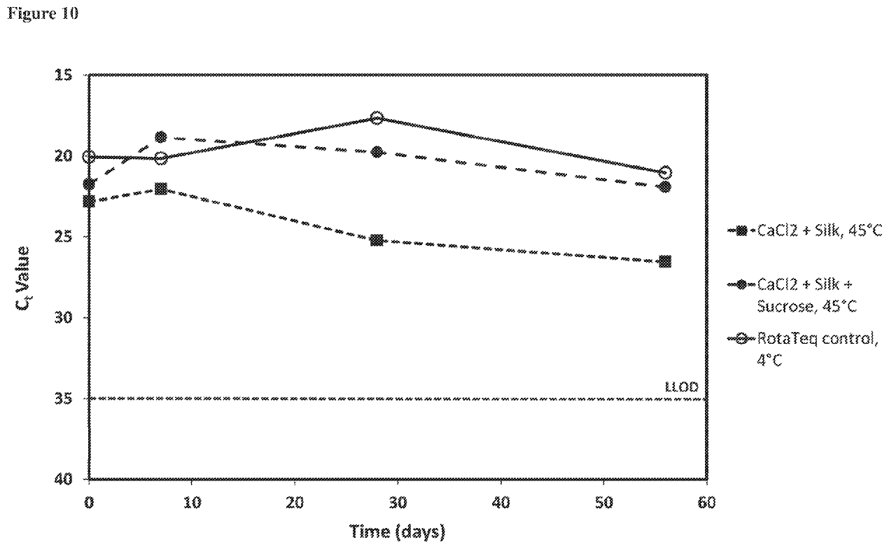

Films were placed on stability at 45° C. Potency was evaluated using RT-PCR at 0, 7, 28, and 56 days. The stability results are depicted in FIG. 10.

### Example 17—Air-Dried Rotavirus Formulation

An exemplary rotavirus formulation prepared using the method described above in Example 11 contained vaccine dialyzed using the method described above in Example 11 and the following excipient mixture in the following final concentration prior to air-drying:
(a) 2.0% w/v of silk fibroin, prepared by the method described above in Example 1,
(b) 10 mM of calcium chloride, and
(c) 12.6 mM of HEPES buffer.

These formulations were cast onto circular molds with 12 mm diameter and dried overnight under ambient conditions (about 20-25° C. and about 30-40% relative humidity). Each mold held 0.1 mL total of vaccine formulation before drying.

Films were placed on stability at 45° C. Potency was evaluated using RT-PCR at 0, 7, 28, and 56 days. The stability results are depicted in FIG. 10.

### Example 18—Air-Dried Rotavirus Formulation

An exemplary rotavirus formulation prepared using the method described above in Example 11 contained vaccine dialyzed using the method described above in Example 11 and the following excipient mixture in the following final concentration prior to air-drying:
(a) 2.0% w/v of silk fibroin, prepared by the method described above in Example 1,
(b) 5.0% w/v of sucrose,
(c) 10 mM of calcium chloride, and
(d) 14.8 mM of HEPES buffer.

These formulations were cast onto circular molds with 12 mm diameter and dried overnight under ambient conditions (about 20-25° C. and about 30-40% relative humidity). Each mold held 0.1 mL total of vaccine formulation before drying.

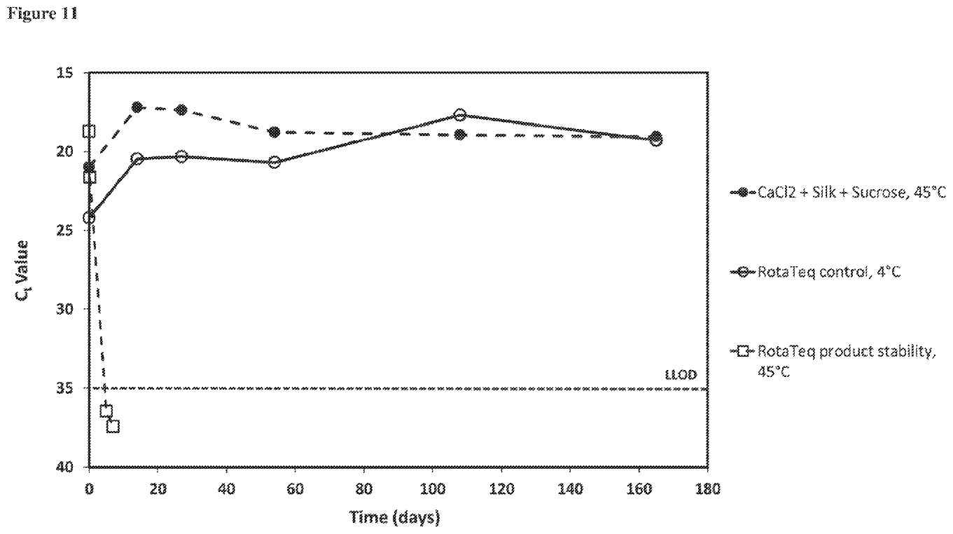

Films were placed on stability at 45° C. Potency was evaluated using RT-PCR at 0, 14, 27, 54, 108, and 165 days. The stability results are depicted in FIG. 11.

### Examples Related to Flavivirus Vaccine Formulations

### Example 19—Hydrolysis of Silk Fibroin

Hydrolyzed silk fibroin solution was prepared according to established methods with modifications for hydrolysis. Briefly, pieces of cocoons from the silkworm Bombyx mori were first boiled in 0.02 M Na2CO3 for 180 minutes to remove sericin protein which is present in unprocessed, natural silk. After rinsing three times in ultrapure water and air-drying overnight, fibroin fibers were solubilized in 10 M HCl at 25° C. for 3 minutes. The solution was subsequently neutralized to a pH of 7 using concentrated NaOH. The subsequent solution was centrifuged to remove any aggregates. The supernatant was isolated, dialyzed against water, and lyophilized. Before use, the lyophilized hydrolyzed silk fibroin was reconstituted in water at the desired concentration.

### Example 20—Preparation of Yellow Fever Vaccine Formulations

### Preparation of Liquid Yellow Fever Vaccine Formulations

Yellow Fever vaccine (YF-Vax®; Sanofi-Pasteur, Lyon, France) was purchased from Henry-Schein (Melville, N.Y., USA). The vaccine was provided as a lyophilized powder hermetically sealed in a vial under nitrogen. To prepare liquid formulations, mixtures of excipients in solution were sterile filtered using a 0.22 μm syringe filter. These excipient mixtures were then added to vials of YF-Vax lyophilized powder, reconstituting the vaccine and resulting in a liquid suspension vaccine formulation.

### Preparation of Dried Yellow Fever Vaccine Formulations

To prepare formulations before drying, mixtures of excipients were sterile filtered using a 0.22 μm syringe filter. These excipient mixtures were then added to vials of YF-Vax lyophilized powder, reconstituting the vaccine and resulting in a liquid suspension vaccine formulation. In the case of formulations that were being prepared for lyophilization, lyophilization was then performed in a Virtis Genesis 25 XL Pilot Lyophilizer (SP Scientific, Gardiner, N.Y., USA). In the case of formulations that were being prepared for air-drying, formulations were then cast onto PDMS molds or into glass vials and allowed to dry, in some cases in a controlled humidity environment, and in some cases in a controlled temperature and pressure environment, such as a lyophilizer.

In the case of formulations that were prepared using an air-drying process followed by a secondary drying process, upon completion of drying at atmospheric conditions, films were removed from PDMS molds and placed into glass vials. At this point, further drying occurred according to a prescribed drying cycle.

### Yellow Fever Vaccine CCID₅₀ Assay

Potency of yellow fever vaccine formulations was measured by viral infectivity in Vero cells (CCID₅₀ assay). Vero cells (CCL-81™, ATTC, Manassas, VA) were diluted to 5×10⁴ cells/mL, plated in 96-well cell culture plates (100 μL/well) and incubated at 37° C./5% CO₂ for one day prior to infection. On the day of infection, yellow fever vaccine samples were reconstituted with diluent (in the case of dried formulations only) and diluted 4-fold serially in cell culture media containing 2% FBS. Vaccine dilutions were added to Vero cell monolayers in the 96-well cell culture plates at 100 μL/well. Typically, 10 replicate wells at each sample dilution were plated with a dilution range spanning 7 to 8 dilutions. Following addition of vaccine dilutions, culture plates were incubated at 37° C./5% CO₂ for 8-10 days. At 8 to 10 days the infectivity of each sample was assessed by reading each well (microscope observation) for signs of cellular cytopathic effects (CPE) induced by the presence of active virus. Viral titer was determined using Spearman-Karber formula (Hamilton et al. (1977), Environmental Science & Technology 11(7):714-9).

### Example 21—Preparation of Japanese Encephalitis Vaccine Formulations

### Preparation of Dried Japanese Encephalitis Vaccine Formulations

Japanese Encephalitis vaccine (IMOJEV®; Sanofi-Pasteur, Lyon, France) was provided as a lyophilized powder sealed in a vial. To prepare formulations before drying, mixtures of excipients were sterile filtered using a 0.22 μm syringe filter. These excipient mixtures were then added to vials of IMOJEV® lyophilized powder, reconstituting the vaccine and resulting in a liquid suspension vaccine formulation. In the case of formulations that were being prepared for air-drying, formulations were then cast onto PDMS molds or into glass vials and allowed to dry, in some cases in a controlled humidity environment, and in some cases in a controlled temperature and pressure environment, such as a lyophilizer.

### Japanese Encephalitis Vaccine CCID₅₀ Assay

Potency of Japanese encephalitis vaccine formulations was measured by viral infectivity in Vero cells (CCID₅₀ assay). Vero cells were diluted to 5×10⁴ cells/mL, plated in 96-well cell culture plates (100 μL/well) and incubated at 37° C./5% CO₂ for one day prior to infection. On the day of infection, Japanese encephalitis vaccine samples were reconstituted with diluent and diluted 4-fold serially in cell culture media containing 2% FBS. Vaccine dilutions were added to Vero cell monolayers in the 96-well cell culture plates at 100 μL/well. Typically, 10 replicate wells at each sample dilution were plated with a dilution range spanning 7 to 8 dilutions. Following addition of vaccine dilutions, culture plates were incubated at 37° C./5% CO₂ for 8-10 days. At 5 to 7 days the infectivity of each sample was assessed by reading each well (microscope observation) for signs of cellular cytopathic effects (CPE) induced by the presence of active virus. Viral titer was determined using Spearman-Karber formula.

### Example 22—Air-Dried Yellow Fever Vaccine Formulation

Exemplary air-dried yellow fever vaccine formulations prepared using the method described above in Example 20 contained one-fifth of a standard dose of YF-Vax® in solution with the following excipient mixture in the following final concentrations prior to air-drying:
(i) 2.5% w/v of silk fibroin, prepared by the method described above in Example 1, and
(j) 5% w/v of sucrose.

These formulations were cast onto circular PDMS molds with 12 mm diameter and air-dried using the following drying cycle:

| Temperature (° C.) | Pressure | Time (minutes) |
|---|---|---|
| 20-25 | Atmospheric | 960 |

Each mold held 0.1 mL total of vaccine formulation before drying.

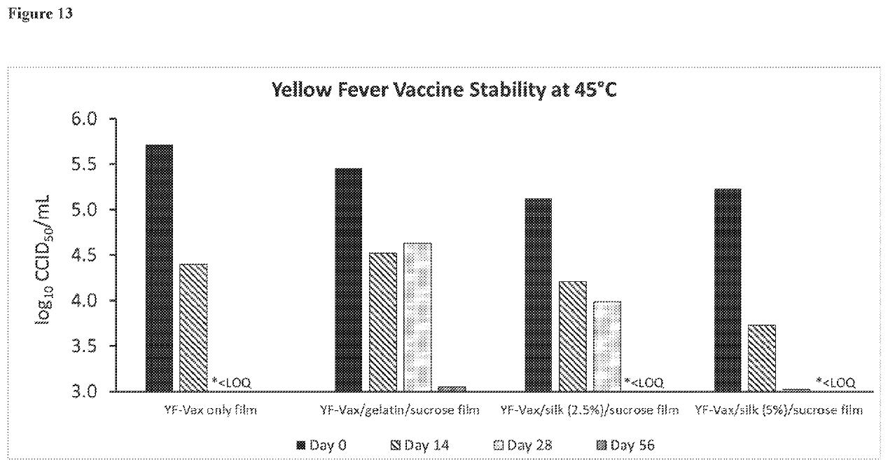

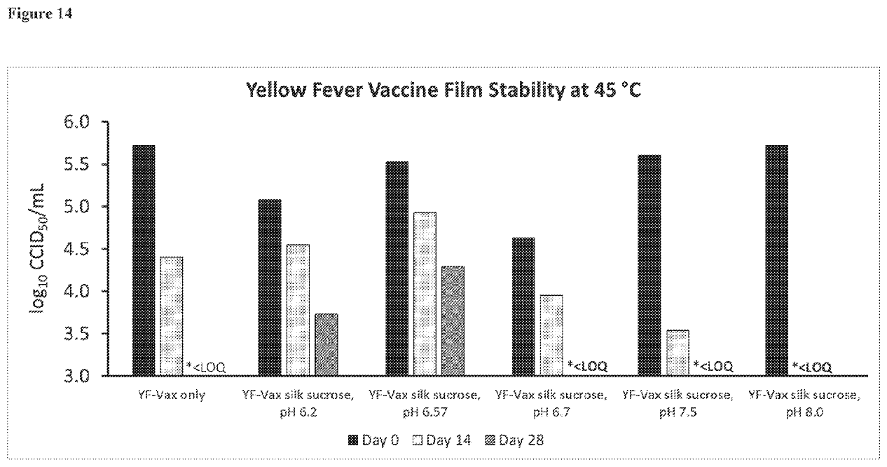

Films (n=2 per temperature and time point) were placed in vials that were back-filled with nitrogen and held in an incubator at 45° C. to assess stability. After reconstitution, potency was evaluated by CCID₅₀ at regular time points. The stability results are depicted in FIGS. 13 and 14.

### Example 23—Air-Dried Yellow Fever Vaccine Formulation

Exemplary air-dried yellow fever vaccine formulations prepared using the method described above in Example 20 contained one-fifth of a standard dose of YF-Vax® in solution with the following excipient mixture in the following final concentrations prior to air-drying:
(a) 2.5% w/v of silk fibroin, prepared by the method described above in Example 1, and
(b) 5% w/v of trehalose.

These formulations were cast onto circular PDMS molds with 12 mm diameter and air-dried using the following drying cycle:

| Temperature (° C.) | Pressure | Time (minutes) |
|---|---|---|
| 20-25 | Atmospheric | 960 |

Each mold held 0.1 mL total of vaccine formulation before drying.

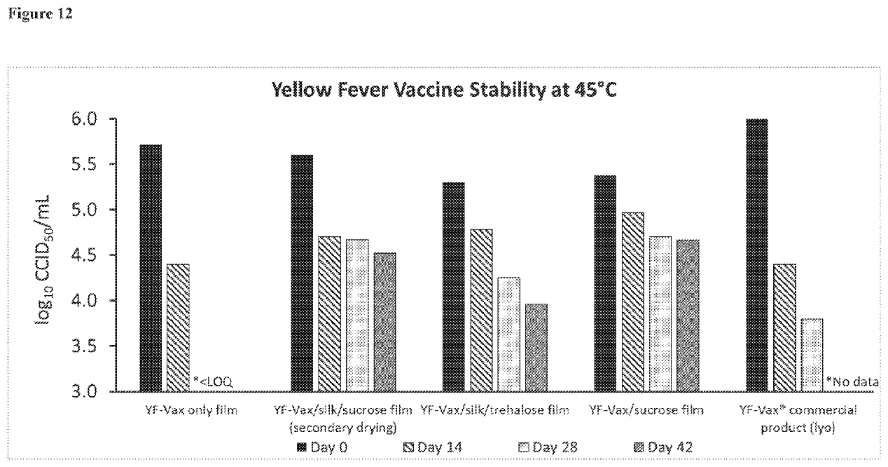

Films (n=2 per temperature and time point) were placed in vials that were back-filled with nitrogen and held in an incubator at 45° C. to assess stability. After reconstitution, potency was evaluated by CCID₅₀ at regular time points. The stability results are depicted in FIG. 12.

### Example 24—Air-Dried Yellow Fever Vaccine Formulation

Exemplary air-dried yellow fever vaccine formulations prepared using the method described above in Example 20 contained one-fifth of a standard dose of YF-Vax® in solution with the following excipient mixture in the following final concentrations prior to air-drying:
(a) 2.5% w/v of gelatin (Gelita VacciPro®, Sergeant Bluff, Iowa, prepared as described above), and
(b) 5% w/v of sucrose.

These formulations were cast onto circular PDMS molds with 12 mm diameter and air-dried using the following drying cycle:

| Temperature (° C.) | Pressure | Time (minutes) |
|---|---|---|
| 20-25 | Atmospheric | 960 |

Each mold held 0.1 mL total of vaccine formulation before drying.

Films (n=2 per temperature and time point) were placed in vials that were back-filled with nitrogen and held in an incubator at 45° C. to assess stability. After reconstitution, potency was evaluated by CCID₅₀ at regular time points. The stability results are depicted in FIG. 13.

### Example 25—Air-Dried Yellow Fever Vaccine Formulation

Exemplary air-dried yellow fever vaccine formulations prepared using the method described above in Example 20 contained one-fifth of a standard dose of YF-Vax® in solution with the following excipient mixture in the following final concentrations prior to air-drying:
(a) 5% w/v of silk fibroin, prepared by the method described above in Example 1, and
(b) 5% w/v of sucrose.

These formulations were cast onto circular PDMS molds with 12 mm diameter and air-dried using the following drying cycle:

| Temperature (° C.) | Pressure | Time (minutes) |
|---|---|---|
| 20-25 | Atmospheric | 960 |

Each mold held 0.1 mL total of vaccine formulation before drying.

Films (n=2 per temperature and time point) were placed in vials that were back-filled with nitrogen and held in an incubator at 45° C. to assess stability. After reconstitution, potency was evaluated by CCID₅₀ at regular time points. The stability results are depicted in FIG. 13.

### Example 26—Air-Dried Yellow Fever Vaccine Formulation with Secondary Drying

Exemplary air-dried yellow fever vaccine formulations prepared with secondary drying using the method described above in Example 4 contained one-fifth of a standard dose of YF-Vax® in solution with the following excipient mixture in the following final concentrations prior to air-drying:
(a) 2.5% w/v of silk fibroin, prepared by the method described above in Example 1, and
(b) 5% w/v of sucrose.

These formulations were cast onto circular PDMS molds with 12 mm diameter and air-dried using the following drying cycle:

| Temperature (° C.) | Pressure | Time (minutes) |
|---|---|---|
| 20-25 | Atmospheric | 960 |

Each mold held 0.1 mL total of vaccine formulation before drying.

After air-drying, films were removed from molds and placed into vials that were back-filled with nitrogen. These formulations then underwent secondary drying in a Virtis Genesis 25 XL Pilot™ Lyophilizer (SP Scientific, Gardiner, NY, USA) using the following drying cycle:

| Temperature (° C.) | Pressure (mT) | Time (minutes) |
|---|---|---|
| 15 | 900 | 30 |
| 15 | 750 | 30 |
| −5 | 50 | 60 |
| 10 | 50 | 120 |
| 20 | 50 | 120 |
| 30 | 50 | 120 |

Vials (n=2 per temperature and time point) were placed in incubators and held at 45° C. to assess stability. After reconstitution, potency was evaluated by CCID₅₀ at regular time points. The stability results are depicted in FIG. 12.

### Example 27—Air-Dried Yellow Fever Vaccine Formulation

Exemplary air-dried yellow fever vaccine formulations prepared using the method described above in Example 20 contained one-fifth of a standard dose of YF-Vax® in solution with the following excipient mixture in the following final concentration prior to air-drying:
(a) 5% w/v of sucrose.

These formulations, which also contained up to 1.5% w/v of protein stabilizer (gelatin) and up to 1.5% w/v of additional sugar alcohol excipient (sorbitol) from the commercial YF-Vax® formulation, were cast onto circular PDMS molds with 12 mm diameter and air-dried using the following drying cycle:

| Temperature (° C.) | Pressure | Time (minutes) |
|---|---|---|
| 20-25 | Atmospheric | 960 |

Each mold held 0.1 mL total of vaccine formulation before drying.

Films (n=2 per temperature and time point) were placed in vials that were back-filled with nitrogen and held in an incubator at 45° C. to assess stability. After reconstitution, potency was evaluated by CCID₅₀ at regular time points. The stability results are depicted in FIG. 12.

### Example 28—Liquid Yellow Fever Vaccine Formulation

Exemplary liquid yellow fever vaccine formulations prepared using the method described above in Example 20 contained one standard dose of YF-Vax® in solution with the following excipient mixture in the following final concentrations:
(a) 4% w/v of silk fibroin, prepared by the method described above in Example 1, and
(b) 0.9% w/v of sodium chloride.

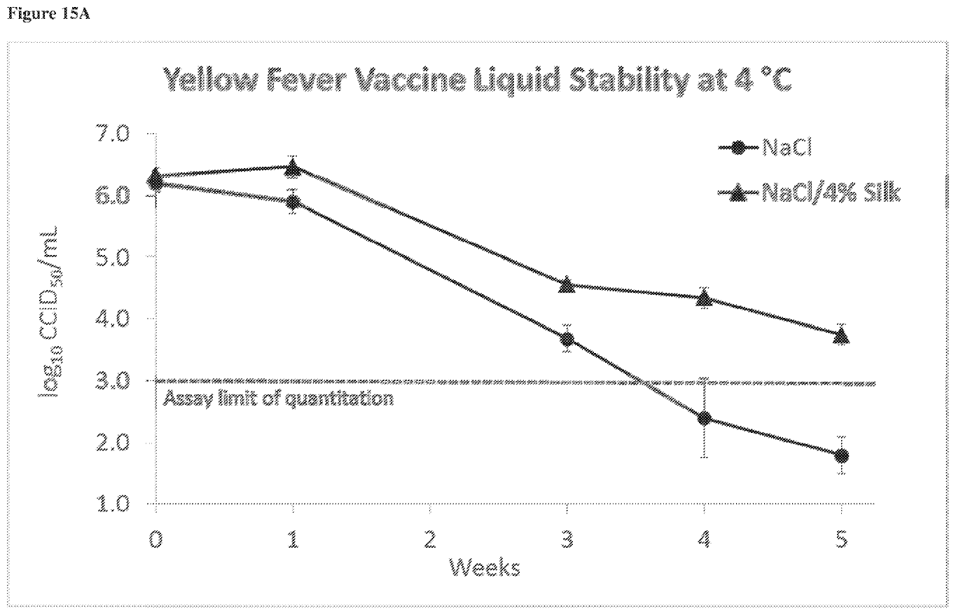

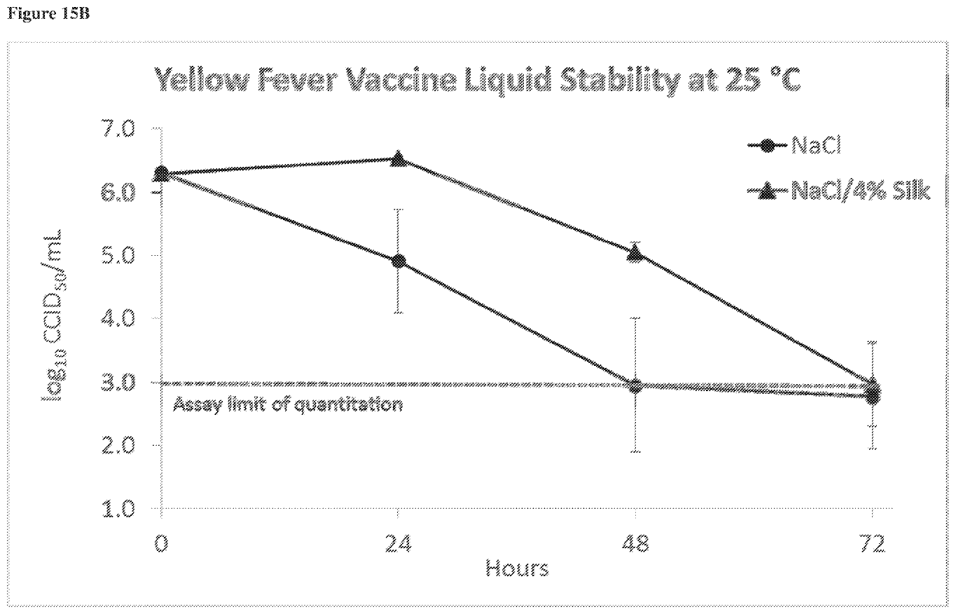

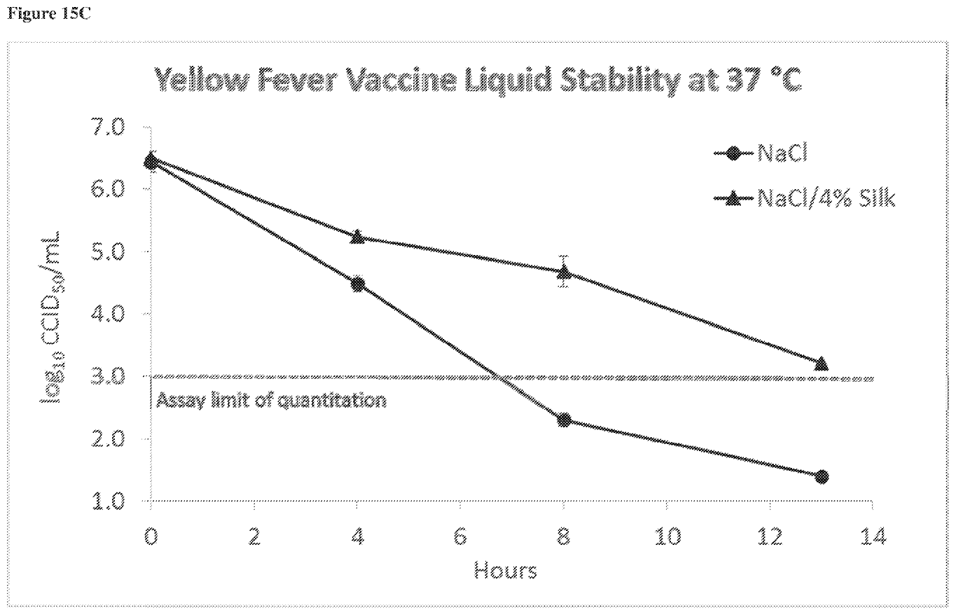

Vials containing the formulation were placed in incubators and held at 4° C., 25° C., and 37° C. to assess stability. Potency of aliquots (n=2 from each vial per temperature and time point) was evaluated by CCID₅₀ at regular time points. The stability results are depicted in FIGS. 15A-15C, 16, and 17.

### Example 29—Liquid Yellow Fever Vaccine Formulation

Exemplary liquid yellow fever vaccine formulations prepared using the method described above in Example 20 contained one standard dose of YF-Vax® in solution with the following excipient mixture in the following final concentrations:
(a) 1% w/v of silk fibroin, prepared by the method described above in Example 1, and
(b) 0.9% w/v of sodium chloride.

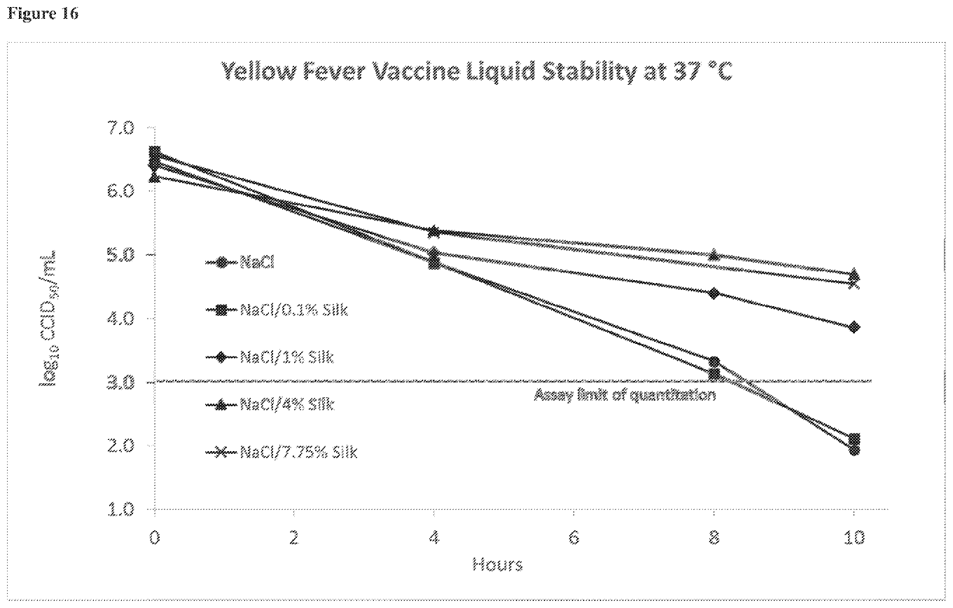

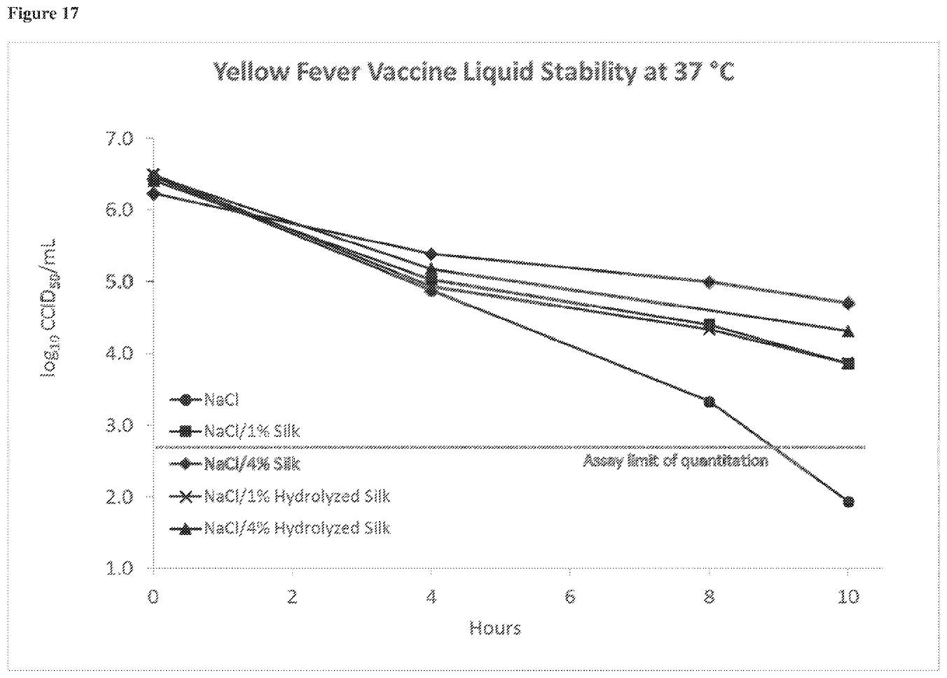

Vials containing the formulation were placed in incubators and held at 37° C. to assess stability. Potency of aliquots (n=2 from each vial per temperature and time point) was evaluated by CCID₅₀ at regular time points. The stability results are depicted in FIGS. 16 and 17.

### Example 30—Liquid Yellow Fever Vaccine Formulation

Exemplary liquid yellow fever vaccine formulations prepared using the method described above in Example 20 contained one standard dose of YF-Vax® in solution with the following excipient mixture in the following final concentrations:
(a) 7.75% w/v of silk fibroin, prepared by the method described above in Example 1, and
(b) 0.9% w/v of sodium chloride.

Vials containing the formulation were placed in incubators and held at 37° C. to assess stability. Potency of aliquots (n=2 from each vial per temperature and time point) was evaluated by CCID₅₀ at regular time points. The stability results are depicted in FIG. 16.

### Example 31—Liquid Yellow Fever Vaccine Formulation

Exemplary liquid yellow fever vaccine formulations prepared using the method described above in Example 20 contained one standard dose of YF-Vax® in solution with the following excipient mixture in the following final concentrations:
(a) 4% w/v of hydrolyzed silk fibroin, prepared by the method described above in Example 19, and
(b) 0.9% w/v of sodium chloride.

Vials containing the formulation were placed in incubators and held at 37° C. to assess stability. Potency of aliquots (n=2 from each vial per temperature and time point) was evaluated by CCID₅₀ at regular time points. The stability results are depicted in FIG. 17.

### Example 32—Liquid Yellow Fever Vaccine Formulation

Exemplary liquid yellow fever vaccine formulations prepared using the method described above in Example 20 contained one standard dose of YF-Vax® in solution with the following excipient mixture in the following final concentrations:
(a) 1% w/v of hydrolyzed silk fibroin, prepared by the method described above in Example 19, and
(b) 0.9% w/v of sodium chloride.

Vials containing the formulation were placed in incubators and held at 37° C. to assess stability. Potency of aliquots (n=2 from each vial per temperature and time point) was evaluated by CCID₅₀ at regular time points. The stability results are depicted in FIG. 17.

### Example 33—Liquid Yellow Fever Vaccine Formulation

Exemplary liquid yellow fever vaccine formulations prepared using the method described above in Example 20 contained one standard dose of YF-Vax® in solution with the following excipient mixture in the following final concentrations:
(a) 0.1% w/v of bovine serum albumin (Sigma-Aldrich, St. Louis, Mo.; product #A3294; prepared as described above), and
(b) 0.9% w/v of sodium chloride.

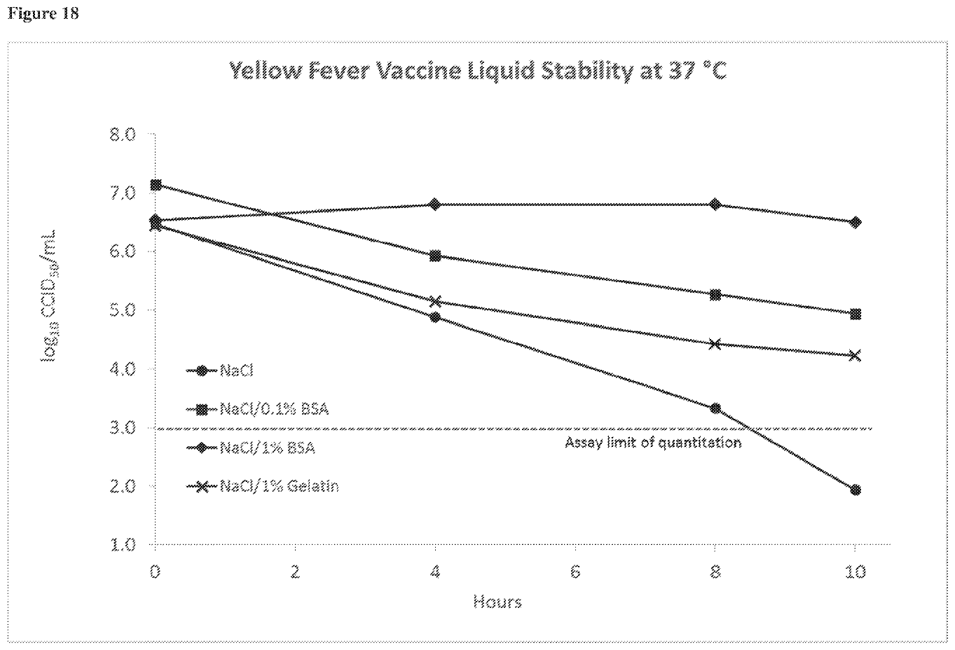

Vials containing the formulation were placed in incubators and held at 37° C. to assess stability. Potency of aliquots (n=2 from each vial per temperature and time point) was evaluated by CCID₅₀ at regular time points. The stability results are depicted in FIG. 18.

### Example 34—Liquid Yellow Fever Vaccine Formulation

Exemplary liquid yellow fever vaccine formulations prepared using the method described above in Example 20 contained one standard dose of YF-Vax® in solution with the following excipient mixture in the following final concentrations:
(a) 1% w/v of bovine serum albumin (Sigma-Aldrich, St. Louis, Mo.; product #A3294; prepared as described above), and
(b) 0.9% w/v of sodium chloride.

Vials containing the formulation were placed in incubators and held at 37° C. to assess stability. Potency of aliquots (n=2 from each vial per temperature and time point) was evaluated by CCID₅₀ at regular time points. The stability results are depicted in FIG. 18.

### Example 35—Liquid Yellow Fever Vaccine Formulation

Exemplary liquid yellow fever vaccine formulations prepared using the method described above in Example 20 contained one standard dose of YF-Vax® in solution with the following excipient mixture in the following final concentrations:
(a) 1% w/v of gelatin (Gelita VacciPro®, Sergeant Bluff, Iowa, prepared as described above), and
(b) 0.9% w/v of sodium chloride.

Vials containing the formulation were placed in incubators and held at 37° C. to assess stability. Potency of aliquots (n=2 from each vial per temperature and time point) was evaluated by CCID₅₀ at regular time points. The stability results are depicted in FIG. 18.

### Example 36—Liquid Yellow Fever Vaccine Formulation

Exemplary liquid yellow fever vaccine formulations prepared using the method described above in Example 20 contained one standard dose of YF-Vax® in solution with the following excipient mixture in the following final concentrations:
(a) 1% w/v of silk fibroin, prepared by the method described above in Example 1,
(b) 0.1% w/v of bovine serum albumin (Sigma-Aldrich, St. Louis, Mo.; product #A3294; prepared as described above), and
(c) 0.9% w/v of sodium chloride.

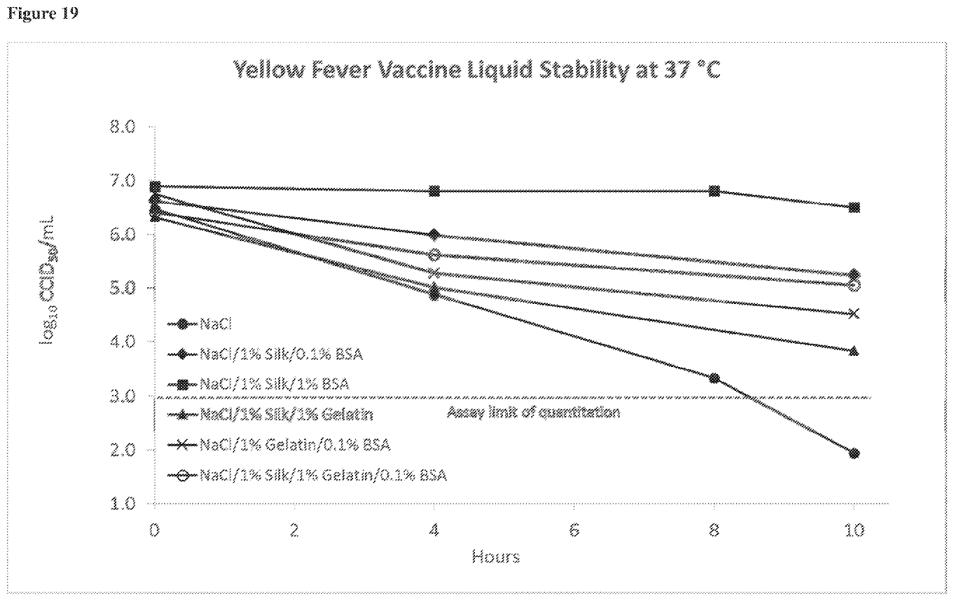

Vials containing the formulation were placed in incubators and held at 37° C. to assess stability. Potency of aliquots (n=2 from each vial per temperature and time point) was evaluated by CCID₅₀ at regular time points. The stability results are depicted in FIG. 19.

### Example 37—Liquid Yellow Fever Vaccine Formulation

Exemplary liquid yellow fever vaccine formulations prepared using the method described above in Example 20 contained one standard dose of YF-Vax® in solution with the following excipient mixture in the following final concentrations:
(a) 1% w/v of silk fibroin, prepared by the method described above in Example 1,
(b) 1% w/v of bovine serum albumin (Sigma-Aldrich, St. Louis, Mo.; product #A3294; prepared as described above), and
(c) 0.9% w/v of sodium chloride.

Vials containing the formulation were placed in incubators and held at 37° C. to assess stability. Potency of aliquots (n=2 from each vial per temperature and time point) was evaluated by CCID₅₀ at regular time points. The stability results are depicted in FIG. 19.

### Example 38—Liquid Yellow Fever Vaccine Formulation

Exemplary liquid yellow fever vaccine formulations prepared using the method described above in Example 20 contained one standard dose of YF-Vax® in solution with the following excipient mixture in the following final concentrations:
(a) 1% w/v of silk fibroin, prepared by the method described above in Example 1,
(b) 1% w/v of gelatin (Gelita VacciPro®, Sergeant Bluff, Iowa, prepared as described above), and
(c) 0.9% w/v of sodium chloride.

Vials containing the formulation were placed in incubators and held at 37° C. to assess stability. Potency of aliquots (n=2 from each vial per temperature and time point) was evaluated by CCID₅₀ at regular time points. The stability results are depicted in FIG. 19.

### Example 39—Liquid Yellow Fever Vaccine Formulation

Exemplary liquid yellow fever vaccine formulations prepared using the method described above in Example 20 contained one standard dose of YF-Vax® in solution with the following excipient mixture in the following final concentrations:
(a) 1% w/v of gelatin (Gelita VacciPro®, Sergeant Bluff, Iowa, prepared as described above),
(b) 0.1% w/v of bovine serum albumin (Sigma-Aldrich, St. Louis, Mo.; product #A3294; prepared as described above), and
(c) 0.9% w/v of sodium chloride.

Vials containing the formulation were placed in incubators and held at 37° C. to assess stability. Potency of aliquots (n=2 from each vial per temperature and time point) was evaluated by CCID₅₀ at regular time points. The stability results are depicted in FIG. 19.

### Example 40—Liquid Yellow Fever Vaccine Formulation

Exemplary liquid yellow fever vaccine formulations prepared using the method described above in Example 20 contained one standard dose of YF-Vax® in solution with the following excipient mixture in the following final concentrations:
(a) 1% w/v of silk fibroin, prepared by the method described above in Example 1,
(b) 1% w/v of gelatin (Gelita VacciPro®, Sergeant Bluff, Iowa, prepared as described above),
(c) 0.1% w/v of bovine serum albumin (Sigma-Aldrich, St. Louis, Mo.; product #A3294; prepared as described above), and
(d) 0.9% w/v of sodium chloride.

Vials containing the formulation were placed in incubators and held at 37° C. to assess stability. Potency of aliquots (n=2 from each vial per temperature and time point) was evaluated by CCID₅₀ at regular time points. The stability results are depicted in FIG. 19.

### Example 41—Air-Dried Japanese Encephalitis Vaccine Formulation

Exemplary air-dried Japanese encephalitis vaccine formulations prepared using the method described above in Example 21 contained one-tenth of a standard dose of IMOJEV® in solution with the following excipient mixture in the following final concentrations prior to air-drying:
(a) 4% w/v of silk fibroin, prepared by the method described above in Example 1.

These formulations, which also contained the protein stabilizer human serum albumin and the sugar alcohol mannitol from the commercial IMOJEV® formulation, were cast onto circular PDMS molds with 12 mm diameter and air-dried using the following drying cycle:

| Temperature (° C.) | Pressure | Time (minutes) |
|---|---|---|
| 20-25 | Atmospheric | 960 |

Each mold held 0.1 mL total of vaccine formulation before drying.

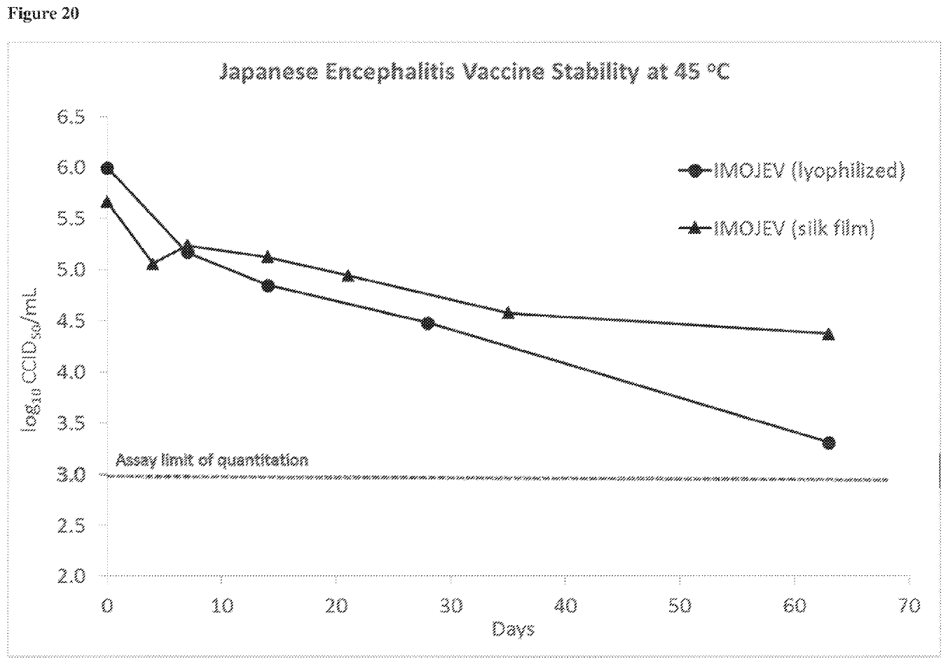

Films (n=2 per temperature and time point) were placed in vials that were back-filled with nitrogen and held in an incubator at 45° C. to assess stability. After reconstitution, potency was evaluated by CCID₅₀ at regular time points. The stability results are depicted in FIG. 20.

### Equivalents.

While this invention has been particularly shown and described with references to preferred embodiments thereof, it will be understood by those skilled in the art that various changes in form and details may be made therein without departing from the spirit and scope of the invention as defined by the appended claims. Those skilled in the art will recognize, or be able to ascertain using no more than routine experimentation, many equivalents to the specific embodiments of the invention described specifically herein. Such equivalents are intended to be encompassed in the scope of the appended claims.

1. A substantially dried viral vaccine preparation comprising:
a viral immunogen;
a protein excipient selected from the group consisting of a silk fibroin, a gelatin and an albumin, or a combination thereof;
a sugar or a sugar alcohol excipient selected from the group consisting of a sucrose, a trehalose, a sorbitol and a glycerol, or a combination thereof;
wherein the vaccine preparation has one, two, three, or four of the following properties:
(i) retains at least 30%, 40%, or 50% of its original bioactivity after storage at 40-45° C. for 3-6 months,
(ii) retains at least 30%, 40%, or 50% of its original bioactivity after storage at 45° C. for 4, 8 or 12 weeks;
(iii) retains at least 30%, 40%, 50% or 60% of its original bioactivity after storage at 37° C. for 4, 8 or 12 weeks; or
(iv) retains at least 70%, 80% or 90% of its original bioactivity after storage at 25° C. for 4, 8, or 12 weeks,
when (i)-(iv) are tested in the vaccine preparation comprising the protein excipient present in an amount of less than 4% (w/v), immediately before drying.

2. The substantially dried viral vaccine preparation of claim 1, wherein the viral immunogen is selected from the group consisting of an enterovirus immunogen, a flavivirus immunogen, a rotavirus immunogen, a measles virus immunogen, a mumps virus immunogen, a rubella virus immunogen, and an influenza virus immunogen.

3.-4. (canceled)

5. The substantially dried viral vaccine preparation of claim 1, which is prepared by air drying, vacuum drying, lyophilization, or partial lyophilization.

6.-8. (canceled)

9. The substantially dried viral vaccine preparation of claim 1, wherein the protein excipient sugar, and/or the sugar alcohol is present in an amount between about 1% (w/v) to about 10% (w/v), immediately before drying.

10.-18. (canceled)

19. The substantially dried viral vaccine preparation of claim 1, further comprising a divalent cation, optionally, selected from the group consisting of Ca²⁺, Mg²⁺, Mn²⁺, and Cu²⁺, optionally wherein the divalent cation is present in the preparation immediately before drying in an amount between 0.1 mM and 100 mM.

20.-22. (canceled)

23. The substantially dried viral vaccine preparation of claim 1, further comprising a buffer, optionally wherein the buffer is selected from the group consisting of a HEPES and a citrate-phosphate (CP) buffer.

24.-30. (canceled)

31. A method of treating or preventing an infection caused by a virus, comprising:
administering to a subject in need thereof an effective amount of a vaccine preparation of claim 1, to treat or prevent the infection.

32. A method of eliciting an immune response to a virus in a subject, comprising:
administering to a subject in need thereof a vaccine preparation of claim 1 in an amount sufficient to elicit the immune response to the virus.

33.-67. (canceled)

68. The substantially dried enterovirus vaccine preparation of claim 2 comprising:
an enterovirus immunogen present in an amount between 0.001 and 20 standard doses;
a silk fibroin present in an amount between 2.0% and 3% (w/v);
a sucrose present in an amount between 4.0% and 6% (w/v), and
a divalent cation, optionally, MgCl₂, present in an amount between 9 mM and 11 mM.

69.-75. (canceled)

76. A liquid stabilized flavivirus vaccine preparation comprising:
a flavivirus immunogen; and
a protein stabilizer,
where the protein stabilizer is chosen from silk fibroin, albumin, gelatin, or a combination thereof.

77.-85. (canceled)

86. The liquid stabilized flavivirus vaccine preparation of claim 76 comprising:
an flavivirus immunogen present in an amount between 0.001 and 20 standard doses;
a silk fibroin present in an amount between 3% and 5% (w/v); and
a salt present in an amount between 0.8% and 10% (w/v).

87.-107. (canceled)

108. The substantially dried flavivirus vaccine preparation of claim 2 comprising:
a flavivirus immunogen present in an amount between 0.001 and 20 standard doses;
a silk fibroin present in an amount between 2% and 3% (w/v); and
a sucrose present in an amount between 4% and 6% (w/v).

109.-127. (canceled)

128. The substantially dried rotavirus vaccine preparation of claim 2 comprising:
a rotavirus immunogen present in an amount between 0.001 and 20 standard doses;
a silk fibroin present in an amount between 1% and 3% (w/v);
a sucrose present in an amount between 4% and 6% (w/v); and
a salt present in an amount between 9 mM and 11 mM.

129.-130. (canceled)

131. A method of preparing a substantially dried viral vaccine preparation of claim 1, optionally a large-scale substantially dried viral vaccine preparation, comprising the steps of:
(i) mixing:
(a) a viral immunogen;
(b) a protein excipient, optionally selected from the group consisting of a silk fibroin, a gelatin and an albumin, or a combination thereof;
(c) a sugar or a sugar alcohol excipient, optionally selected from the group consisting of a sucrose, a trehalose, a sorbitol and a glycerol, or a combination thereof; and
(d) optionally, a divalent cation,
thereby forming a vaccine mixture, and
(ii) lyophilizing or drying, optionally, air drying, the vaccine mixture at about 2° C. to about 50° C., optionally at about 20° C. to about 25° C., and optionally at about 20% to about 40% relative humidity, thereby a large-scale formulation is prepared at about 1-million dosage units per year.

132.-135. (canceled)
